(19) United States
(12) Patent Application Publication
(10) Pub. No.: US 2025/0034202 A1
(43) Pub. Date: Jan. 30, 2025

(54) CELASTROL PROTEOLYSIS TARGETING CHIMERAS

(71) Applicants: The General Hospital Corporation, Boston, MA (US); President and Fellows of Harvard College, Cambridge, MA (US)

(72) Inventors: Ralph Mazitschek, Weston, MA (US); Connor Payne, Boston, MA (US); Bakhos Tannous, Lynnefield, MA (US)

(21) Appl. No.: 18/708,967

(22) PCT Filed: Nov. 10, 2022
(86) PCT No.: PCT/US2022/049540

§ 371 Date: May 9, 2024

Related U.S. Application Data
(60) Provisional application No. 63/391,655, filed on Jul. 22, 2022.
(60) Provisional application No. 63/277,643, filed on Nov. 10, 2021.

(51) Int. Cl. C07J 63/00; A61K 31/551; A61K 31/58; A61P 35/00

(52) U.S. Cl. CPC C07J 63/008; A61K 31/551; A61K 31/58; A61P 35/00

(57) ABSTRACT

The present application provides compounds that are proteolysis targeting chimeras containing a celastrol-based ubiquitin ligase targeting moiety. Methods of using these compounds for treating various diseases, such as neurodegenerative diseases and cancer, are also provided.

### CLAIM OF PRIORITY

This application claims priority to U.S. Patent Application Ser. No. 63/391,655, filed on Jul. 22, 2022, and 63/277,643, filed on Nov. 10, 2021, the entire contents of which are hereby incorporated by reference.

### TECHNICAL FIELD

This invention relates to bi-functional conjugates (PROTACs, or proteolysis targeting chimeras) containing at least three elements: (i) a targeting ligand capable of selectively binding a protein for degradation; (ii) a celastrol-based ubiquitin ligase recruiting ligand; and (iii) a linker between the ubiquitin ligase (ii) and the targeting ligand (i).

### BACKGROUND

Cancer is one of the leading causes of death in contemporary society. The numbers of new cancer cases and deaths is increasing each year. Currently, cancer incidence is nearly 450 cases of cancer per 100,000 men and women per year, while cancer mortality is nearly 71 cancer deaths per 100,000 men and women per year. Likewise, other diseases, such as inflammatory conditions, neurodegenerative diseases, anemia, ischemia, and diabetes affect a significant segment of population. For example, Alzheimer's disease (AD), a neurodegenerative tauopathy that affects approximately 44 million people world-wide, is the sixth leading cause of death with an estimated socioeconomic burden of more than $200 billion.

### SUMMARY

In one general aspect, the present disclosure provides a proteolysis targeting chimera (PROTAC) comprising (i) a targeting ligand capable of selectively binding a protein for degradation; (ii) a celastrol-based ubiquitin ligase recruiting ligand; and (iii) a linker between the ubiquitin ligase (ii) and the targeting ligand (i). Without being bound by any theory, it is believed that the PROTAC of this disclosure allows to degrade a protein implicated in the pathology of a disease or condition using proteasome machinery of a cell, advantageously leading to favorable therapeutic outcomes.

In one general aspect, the present disclosure provides a compound of Formula (I):

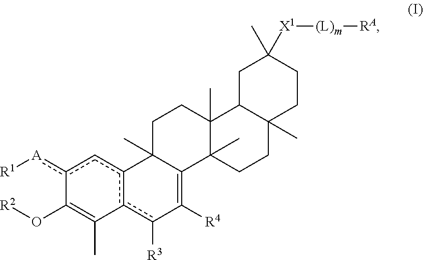

or a pharmaceutically acceptable salt thereof, wherein X¹, R¹, R², R³, R⁴, m, L, and R^{A} are as described herein.

In one general aspect, the compound of Formula (I) has formula:

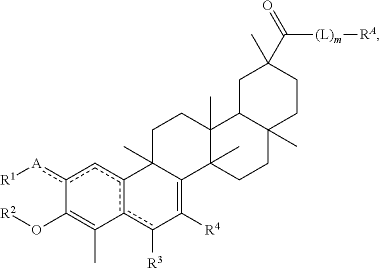

or a pharmaceutically acceptable salt thereof, wherein R¹, R², R³, R⁴, m, L, and R^{A} are as described herein.

In one general aspect, the compound of Formula (I) has formula:

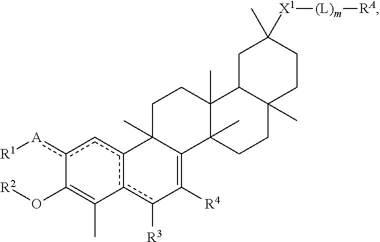

or a pharmaceutically acceptable salt thereof, wherein R¹, R², R³, R⁴, m, L, and R^{A} are as described herein.

In some embodiments, the compound of Formula (I) has formula:

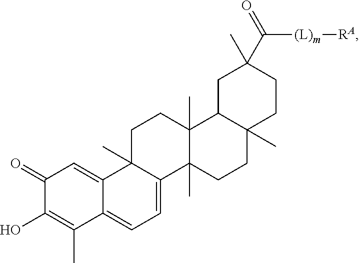

or a pharmaceutically acceptable salt thereof, wherein L, m, and R^{A} are as described herein.

In some embodiments, the compound of Formula (I) has formula:

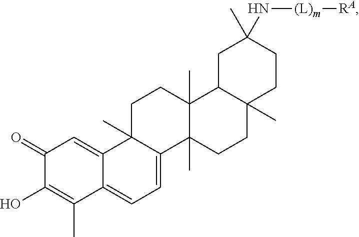

or a pharmaceutically acceptable salt thereof, wherein L, m, and R^{A} are as described herein.

In another general aspect, the present disclosure provides a pharmaceutical composition comprising the compound of Formula (I), or a pharmaceutically acceptable salt thereof, and a pharmaceutically acceptable carrier.

In yet another general aspect, the present disclosure provides a method of treating a disease or condition, the method comprising administering to a subject in need thereof a therapeutically effective amount of a compound of Formula (I), or a pharmaceutically acceptable salt thereof. In some embodiments, the method comprises administering to the subject a combination of the compound of Formula (I), or a pharmaceutically acceptable salt thereof, and a second therapeutic agent, or a pharmaceutically acceptable salt thereof.

In another general aspect, the present disclosure provides a method of treating cancer, the method comprising administering a subject in need thereof:
(i) a therapeutically effective amount of celastrol having formula:

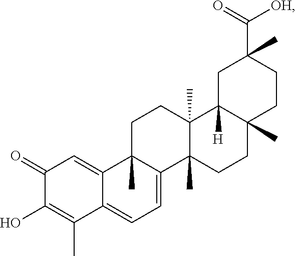

or a pharmaceutically acceptable salt thereof, and
(ii) a therapeutically effective amount of a bromodomain inhibitor, or a pharmaceutically acceptable salt thereof.

Unless otherwise defined, all technical and scientific terms used herein have the same meaning as commonly understood by one of ordinary skill in the art to which the present application belongs. Methods and materials are described herein for use in the present application; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the present application will be apparent from the following detailed description and figures, and from the claims.

### DESCRIPTION OF DRAWINGS

FIG. 1A shows that celastrol is a powerful E3 ubiquitin ligase recruiter for targeted protein degradation applications. The figure shows chemical structures of celastrol (CS) and celastrol-JQ1 (CS-JQ1; 2). Thiophilic sites are highlighted with grey circles.

FIG. 1B shows quantification of target engagement of CS-JQ1 with recombinant BRD4(BD1), BRD4(BD2), and endogenous BRD4 in MCF7 cell extracts (n=2). Data were fitted to a four-parameter dose-response model in Prism 9 (n=2, Kelch; n=4, BTB).

FIG. 1C shows dose-titration of CS and CS-JQ1 in TR-FRET assays with full-length Keap1 (see STAR Methods). Data were fitted to a four-parameter dose-response model in Prism 9 (n=2, Kelch; n=4, BTB).

FIG. 1D shows CS-JQ1-induced ternary complex formation between full-length Keap1 and BRD4(BD1) and BRD4(BD2) (n=2).

FIG. 1E shows TR-FRET quantification of BRD4 levels in MCF7 and MDA-MB-231 cells after treatment with dose-titrations of CS-JQ1 for 5 h in 24-well plate assay format. The DC_{50,5h} and E_{max,5h} values for CS-JQ1 were 29±14 (21%) and 16±2 nM (12%) in MCF7 and MDA-MB-231 cells, respectively (n=2).

FIG. 1F shows western blot analysis of the same samples used for TR-FRET quantification in FIG. 1E.

FIG. 1G shows western blot analysis of the same samples used for TR-FRET quantification in FIG. 1E.

FIG. 1H shows rescue of CS-JQ1-induced BRD4 degradation (250 nM) by BTZ, MLN7243, MLN4924 (1 μM) and JQ1 (10 μM) in MCF7 cells (n=2).

FIG. 1I shows rescue of CS-JQ1-induced BRD4 degradation (250 nM) by BTZ, MLN7243, MLN4924 (1 μM) and JQ1 (10 μM) in MDA-MB-231 cells (n=2).

FIG. 1J shows that co-treatment of MDA-MB-231 cells with CS-JQ1 (250 nM) and potent BTB-targeting Keap1 ligand CDDO-Me (1 μM) does not attenuate BRD4 degradation, indicating potential activity mediated through additional E3 ligase complexes other than Keap1/CRL3 (n=2). Data in FIG. 1B-FIG. 1D are expressed as mean±SD of n technical replicates and are representative of at least two independent experiments. Data in FIG. 1E-FIG. 1J are expressed as mean±SD of n biological replicates.

FIG. 2 contains chemical structures of CDDO and CDDO-Me. Thiophilic site is shown as a grey circle.

FIG. 3 contains a table showing apparent equilibrium dissociation constants for individual recombinant bromodomains and endogenous BRD4 determined by biochemical TR-FRET ligand displacement assays.

FIG. 4 contains a table showing cellular degradation constants for small molecule BRD4 degraders determined by TR-FRET.

FIG. 5 contains a table showing apparent equilibrium dissociation constants for CS and CS-JQ1 toward Keap1-Kelch and Keap1-BTB domains.

FIG. 6A shows results of treating MDA (breast cancer) cells (passage 40, 25 μg of protein) with low CDDO (100 nM), high CDDO (5 μm), celastrol-JQ1 (100 nM), low CDDO and celastrol-JQ1, and high CDDO and celastrol-JQ1.

FIG. 6B shows results of treating MES83 (glioma) cells (passage 7, 30 μg of protein) with low CDDO (100 nM), high CDDO (5 μm), celastrol-JQ1 (100 nM), low CDDO and celastrol-JQ1, and high CDDO and celastrol-JQ1.

FIG. 7A shows BRD4 degradation after treating MES83 (glioma) cells with JQ1, celastrol, and celastrol-JQ1.

FIG. 7B shows BRD4 degradation after treating GBM8 (glioma) cells with JQ1, celastrol, and celastrol-JQ1.

FIG. 7C shows BRD4 degradation after treating DIPG XIII (pediatric glioma) cells with JQ1, celastrol, and celastrol-JQ1.

FIG. 7D shows BRD4 degradation after treating MDA-MB-BR (metastatic breast cancer) cells with JQ1, celastrol, and celastrol-JQ1.

FIG. 7E shows BRD4 degradation after treating BT474 (breast cancer) cells with JQ1, celastrol, and celastrol-JQ1.

FIG. 8 shows results of BRD4 degradation at various concentrations of celastrol-JQ1 and obtusaquinone (OBT)-JQ1. All samples were pre-treated with cicloheximide CHX (3 μg/ml) for 1 h.

FIG. 9A shows BRD4 degradation in different glioma cells by MG132 and CS-JQ1.

FIG. 9B shows BRD4 degradation in different pediatric glioma cells by MG132 and CS-JQ1.

FIG. 9C shows BRD4 degradation in different breast cancer cells by MG132 and CS-JQ1.

FIG. 10A shows relative cell viability for cells treated with either JQ-1 or a combination of JQ-1 and celastrol.

FIG. 10B shows relative cell viability for cells treated with either control or celastrol.

### DETAILED DESCRIPTION

Over the past decade, the small molecule-induced selective degradation of specific protein targets has emerged as a novel modality in biomedical research that holds promise to revolutionize drug development, providing access to new classes of therapeutics that can act on disease-relevant proteins previously considered to be undruggable. The concept of targeted protein degradation (TPD) is based on the ligand-induced recruitment of a protein of interest (POI) to an E3 ubiquitin ligase complex. Provided productive orientation, the POI is ubiquitinated and consecutively eliminated by proteasomal degradation.

There are two fundamental classes of small molecules that are employed for TPD, commonly referred to as “molecular glues” and PROTACs (proteolysis-targeting chimeras). Molecular glues are monofunctional ligands, which stabilize the interaction between two proteins that would otherwise not bind. In TPD, molecular glues can enable the recruitment of POIs as neosubstrates to E3 ligase complexes. PROTACs, in contrast, are heterobifunctional molecules that are formally composed of two individual ligands, one for binding the E3 ligase complex and one for targeting the POI, that are tethered by a linker. PROTACs are particularly attractive because the modular design facilitates rational development and optimization by systematic variation of the three elements.

Despite the large number of E3 ligases (e.g., >600 E3 ligases in the human proteome) that could potentially be employed, early PROTAC development approaches have predominantly focused on a multitude of POIs and largely relied on only few E3 ligases (e.g. VHL and cereblon) that are targeted by a small number of ligand classes. Although the specific POIs vary between studies, BRD4 (bromodomain-containing protein 4) is often used as proof-of-concept in PROTAC development programs that explore new E3 ligase binders and/or linker elements. The reason for this is likely a combination of several factors, including historic reference data, abundance, ubiquitous expression, robustness of response, validated orthogonal inhibitor classes, and more recently the availability of commercial building blocks. Only more recently efforts have intensified to identify novel recruiting elements that broaden the scope of tractable E3 ligases.

Recently, the targeting of other E3 ubiquitin ligase complexes, apart from those canonically used such as CRBN and VHL, using (reversible) covalent ligands that target cysteine side chains has gained increasing attention. Of the predicted >600 E3 ligases, a substantial fraction feature solvent-exposed cysteine residues that can potentially be exploited with corresponding sulfhydryl-reactive biasing elements, including the recently targeted RNF114, RNF4, FEM1B, DCAF16, and DCAF11 complexes.

While the selective targeting of E3 ligases can potentially provide cell type- or tissue-specific degradation, targeting of multiple ligase complexes with a “promiscuous” reversible covalent inhibitor provides a means for efficient TPD that is indiscriminate to cell and tissue types. For example, a bardoxolone methyl (CDDO-Me) is a synthetic triterpenoid that binds with high affinity the Keap1 (Kelch-like ECH-associated protein 1), a redox-regulated member of a CRL3 (Cullin-RING E3 ligase) complex, by forming reversible covalent adducts with multiple cysteine nucleophiles within the protein. A PROTAC (e.g., CDDO-JQ1) derived from bardoxolone methyl (CDDO-Me) was used to target various E3 ligases containing cysteine side chains that are reactive with the CDDO recruiter. The triterpenoid celastrol ((9β,13α,14β,20α)-3-hydroxy-9,13-dimethyl-2-oxo-24,25,26-Trinoroleana-1(10),3,5,7-tetraen-29-oic acid, CAS Reg. No. 34157-83-0):

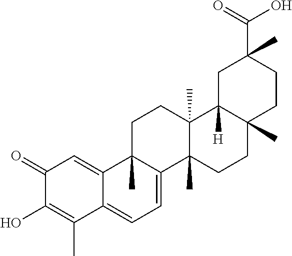

can also form reversible covalent adducts with multiple cysteine nucleophiles and has been shown to bind a host of proteins. Like CDDO, for example, celastrol targets Keap1, by binding Keap1 BTB and Kelch domains with low micromolar affinity. In one general aspect, this disclosure provides next generation PROTAC compounds using celastrol, a triterpene natural product that reversibly and covalently binds cysteine side chains of multiple proteins, as a powerful indiscriminate E3 ligase recruiter for a multitude of proteins of interest that are implicated in a variety of diseases and conditions, such as cancer (e.g., prostate cancer, breast cancer, or leukemia), prion diseases (e.g., encephalopathy), and tauopathies (e.g., Alzheimer's disease, Pick disease, frontotemporal dementia, or Parkinson's disease). Pharmaceutical compositions and methods of using these PROTAC compounds for treating the aforementioned diseases, alone or in combination with another therapeutic agent, are also provided. Some embodiments, of the compounds, pharmaceutical compositions, methods of treatment (including combination treatments) are described herein.

### Therapeutic PROTAC Compounds

In some embodiments, the present application provides a compound useful as a PROTAC (a proteolysis targeting chimera). Generally, PROTACS are bi-functional conjugates containing three elements: (i) a targeting ligand capable of selectively binding a protein for degradation; (ii) a ubiquitin ligase recruiting ligand; and (iii) a linker between the ubiquitin ligase (ii) and the targeting ligand (i). In one general aspect, in the PROTACS of the present disclosure, the ubiquitin ligase recruiting ligand (ii) is a celastrol or a derivative thereof. Generally, celastrol is a triterpenoid compound having the following chemical structure:

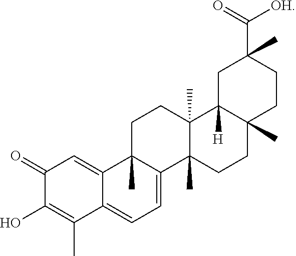

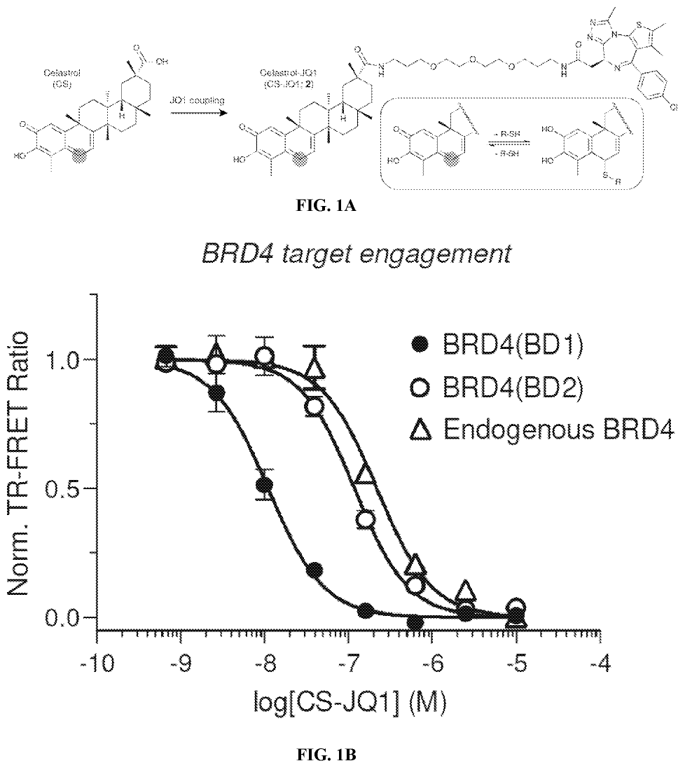

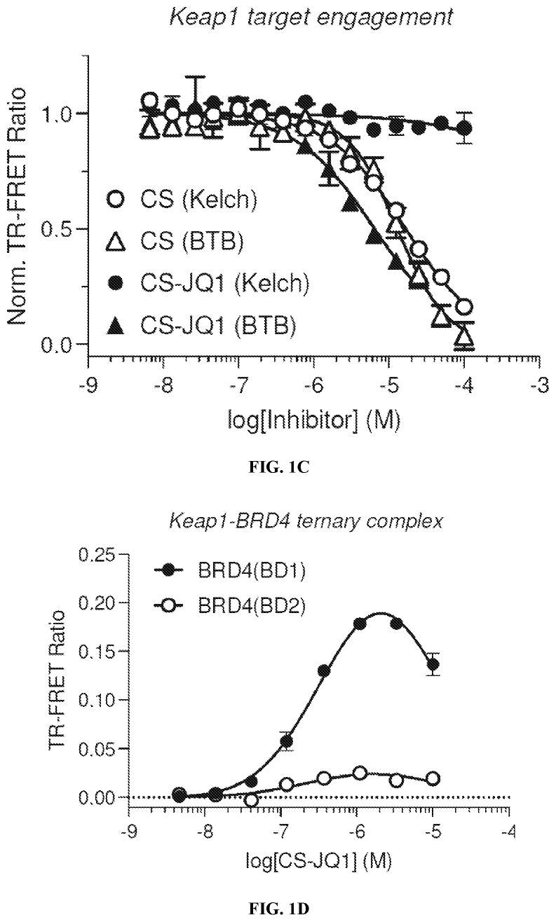

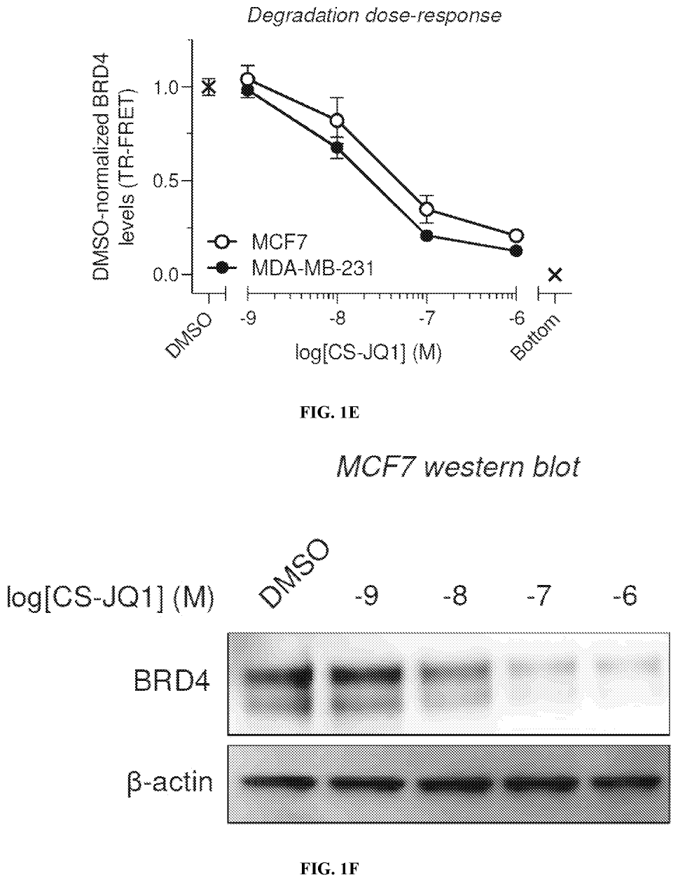

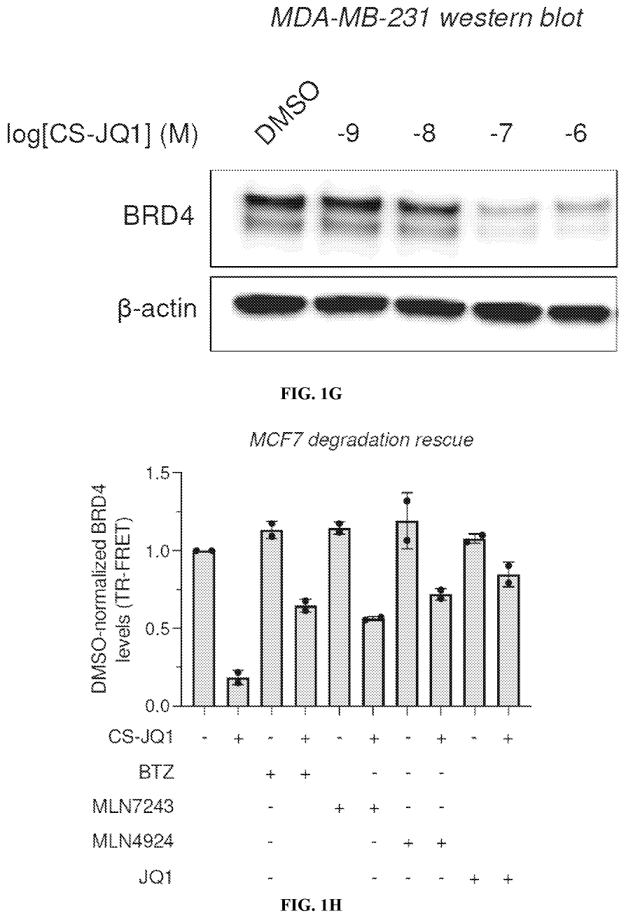

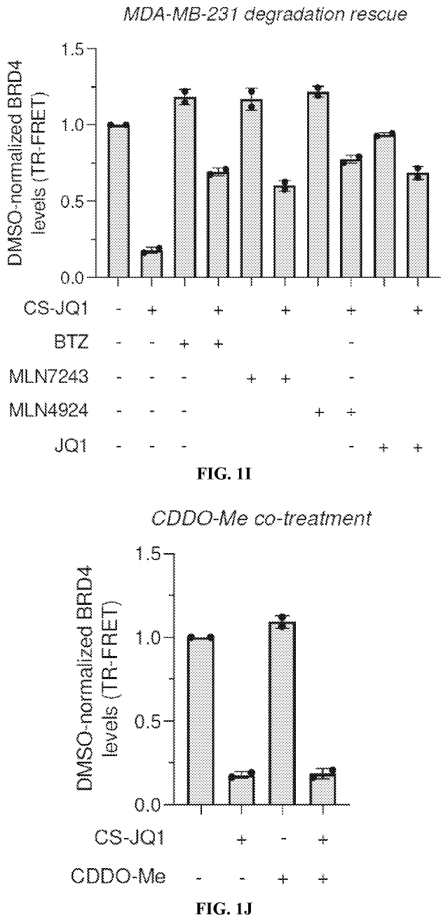

Alternative chemical names for celastrol include (9β,13α,14β,20α)-3-Hydroxy-9,13-dimethyl-2-oxo-24,25,26-trinoroleana-1(10),3,5,7-tetraen-29-oic acid, 3-hydroxy-2-oxo-24-nor-D:A-friedooleana-1(10),3,5,7-tetraen-29-oic acid, (2R,4aS,6aS,12bR,14aS,14bR)-1,2,3,4,4a,5,6,6a,11,12b,13,14,14a,14b-tetradecahydro-10-hydroxy-2,4a,6a,9,12b,14a-hexamethyl-11-oxo-2-picenecarboxylic acid, NSC70931, tripterin, and UI1590. CAS Reg. No. for celastrol is 34157-83-0. Molecular weight of celastrol is about 450.6 g/mol, melting point is about 195° C., and a pKa is about 4.8. Without being bound by any particular theory, it is believed that the celastrol-containing moiety in the PROTACs of this discloser binds to E3 ligase from RING/U-box family, active-site cysteine-containing HECT family, RING-in-Between-RING (RBR) family, or any combination of the foregoing. The binding may be covalent or non-covalent (e.g., through hydrogen bonds, electrostatic interactions, hydrophobic interactions, van der Waals interactions, or any combinations of the foregoing), reversible or irreversible. In some embodiments, the binding of the celastrol-containing moiety to the ligase is a reversible covalent binding. An exemplary mode of reversible covalent binding of celastrol with the cysteine side-chains of an E3 ligase is schematically shown in FIG. 1A. In some embodiments, the celastrol-containing moiety binds with an E3 ligase selected from von Hippel Lindau (VHL), cereblon, XIAP, cIAP, Keap1, RNF4, RNF114, and MDM2, or any combination of the foregoing.

In some embodiments, the PROTAC compound of this disclosure comprises any one of the celastrol-based moieties, and prodrugs thereof, described in US20190062254 and US20210061851, which are incorporated herein by reference in their entirety.

In one general aspect, the present disclosure provides a compound of Formula (I):

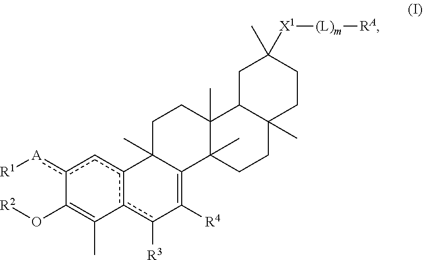

or a pharmaceutically acceptable salt thereof, wherein:
[symbol] indicates a single bond or a double bond;
A is N or O when the bond between A and the carbon to which A is attached is a double bond; or
A is O when the bond between A and the carbon to which A is attached is a single bond;
R¹ is absent when A is O and the bond between A and the carbon to which A is attached is a double bond;
R¹ is H or OH when A is N and the bond between A and the carbon to which A is attached is a double bond; or
R¹ is selected from H, R⁵, COR⁵, COOR⁵, and CONHR⁵, when the bond between A and the carbon to which A is attached is a single bond;
R² is selected from H, R⁵, COR⁵, COOR⁵, and CONHR⁵;
R³ is selected from H, halo, NO₂, CN, OH, SH, OR⁶, SR⁶, C₁₋₆ alkyl, C₁₋₆ haloalkyl, amino, C₁₋₆ alkylamino, di(C₁₋₆ alkyl)amino, CONH₂, CONH(C₁₋₆ alkyl), CON(C₁₋₆ alkyl)₂, COOH, and CO(OC₁₋₆ alkyl);
R⁴ is selected from H, OH, H, halo, NO₂, CN, OH, C₁₋₆ alkyl, C₁₋₆ haloalkyl, C₁₋₆ alkoxy, C₁₋₆ haloalkoxy, amino, C₁₋₆ alkylamino, di(C₁₋₆ alkyl)amino, CONH₂, CONH(C₁₋₆ alkyl), CON(C₁₋₆ alkyl)₂, COOH, and CO(OC₁₋₆ alkyl);
each R⁵ is independently selected from C₁₋₆ alkyl and C₆₋₁₀ aryl, each of which is optionally substituted with 1 or 2 substituents independently selected from NH₂, halo, OH, CN, NO₂, C₁₋₃ alkoxy, OCOC₁₋₆ alkyl, COOH, and CO(OC₁₋₆ alkyl);
each R⁶ is independently selected from R⁵ and COR⁵;
m is an integer from 2 to 20;
each L is independently selected from N(R^{N}), O, C(═O), S, S(═O), S(═O)₂, C₁₋₆ alkylene, C₃₋₇ cycloalkylene, 4-10-membered heterocycloalkylene, 5-10-membered heteroarylene, C₆₋₁₀ arylene, —(OCH₂CH₂)ₓ—, —(CH₂CH₂O)ₓ—, —(OCH(CH₃)CH₂)ₓ—, —(CH₂CH(CH₃)O)—, each of which is optionally substituted with 1 or 2 substituents independently selected from OH, NH₂, C(O)OH, SO₃H, C₁₋₃ alkylamino, di(C₁₋₃-alkyl)amino, C₁₋₃ haloalkyl, C₁₋₃ alkoxy, and C₁₋₃ haloalkoxy;
each x is independently an integer from 1 to 2,000;
each R^{N} is independently selected from H, C₁₋₆ alkyl, C₂₋₆ alkenyl, and C₂₋₆ alkynyl; and
R^{A} is a targeting ligand capable of selectively binding to a protein.

In some embodiments, [symbol] indicates a single bond.

In some embodiments, [symbol] indicates a double bond.

In some embodiments, A is N. In some embodiments, A is O.

In some embodiments, A is N, the bond between A and the carbon to which A is attached is a double bond, and R¹ is H or OH. In some embodiments, A is O, the bond between A and the carbon to which A is attached is a double bond, and R¹ is absent.

In some embodiments, A is O, the bond between A and the carbon to which A is attached is a single bond, and R¹ is selected from H, R⁵, COR⁵, COOR⁵, and CONHR⁵. In some aspects of these embodiments, R¹ is H. In some aspects of these embodiments, R¹ is R⁵. In some aspects of these embodiments, R¹ is COR⁵. In some aspects of these embodiments, R¹ is COOR⁵. In some aspects of these embodiments, R¹ is CONHR⁵.

In some embodiments, R² is H. In some embodiments, R² is R⁵. In some embodiments, R² is COR⁵. In some embodiments, R² is COOR⁵. In some embodiments, R² is CONHR⁵.

In some embodiments, R³ is H. In some embodiments, R³ is OR⁶. In some embodiments, R³ is SR⁶.

In some embodiments, R⁴ is H. In some embodiments, R⁴ is C₁₋₆ alkyl.

In some embodiments, R⁵ is C₁₋₆ alkyl, optionally substituted with 1 or 2 substituents independently selected from NH₂, halo, OH, CN, NO₂, C₁₋₃ alkoxy, OCOC₁₋₆ alkyl, COOH, and CO(OC₁₋₆ alkyl). In some embodiments, R⁵ is C₁₋₆ alkyl, optionally substituted with NH₂, OH, C₁₋₃ alkoxy, COOH, or CO(OC₁₋₆ alkyl).

In some embodiments, R⁵ is C₆₋₁₀ aryl, optionally substituted with 1 or 2 substituents independently selected from NH₂, halo, OH, CN, NO₂, C₁₋₃ alkoxy, OCOC₁₋₆ alkyl, COOH, and CO(OC₁₋₆ alkyl).

In some embodiments, R⁶ is R⁵. In some embodiments, R⁶ is COR⁵.

In some embodiments, the compound has formula:

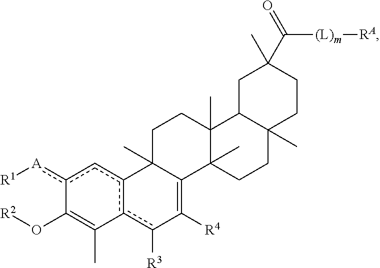

or a pharmaceutically acceptable salt thereof.

In some embodiments, the compound has formula:

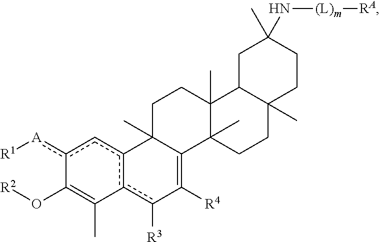

or a pharmaceutically acceptable salt thereof.

In some embodiments, the compound has formula:

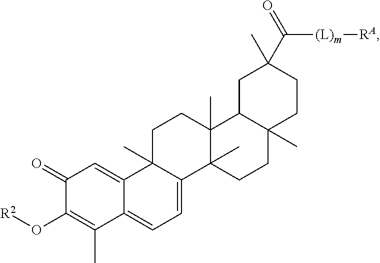

or a pharmaceutically acceptable salt thereof.

In some embodiments, the compound has formula:

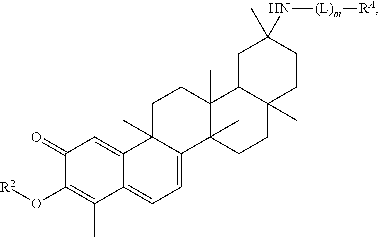

or a pharmaceutically acceptable salt thereof.

In some embodiments, R⁵ is C₁₋₄ alkyl, optionally substituted with NH₂, OH, C₁₋₃ alkoxy, COOH, or CO(OC₁₋₄ alkyl).

In some embodiments, the compound has formula:

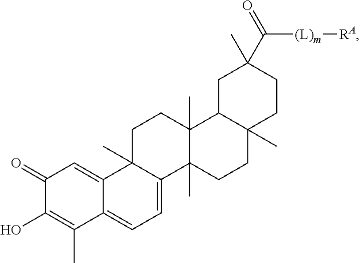

or a pharmaceutically acceptable salt thereof.

In some embodiments, the compound has formula:

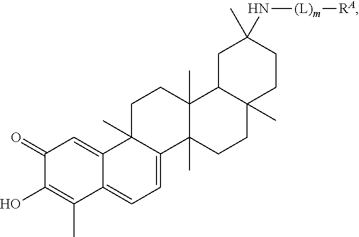

or a pharmaceutically acceptable salt thereof.

In some embodiments, the compound has formula:

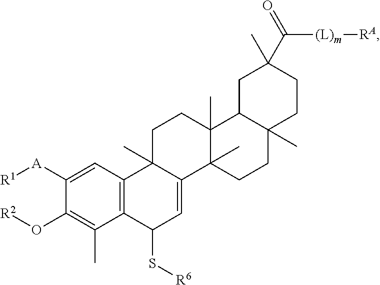

or a pharmaceutically acceptable salt thereof.

In some embodiments, the compound has formula:

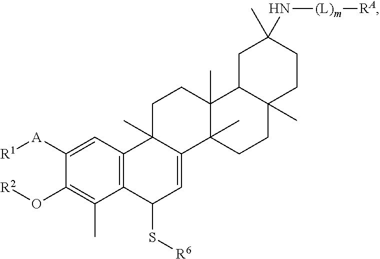

or a pharmaceutically acceptable salt thereof.

In some embodiments:
R¹ is COR⁵
R² is COR⁵;
R⁶ is R⁵; and
R⁵ is C₁₋₆ alkyl, optionally substituted with NH₂, OH, or COOH.

In some embodiments, the compound of Formula (I) has formula:

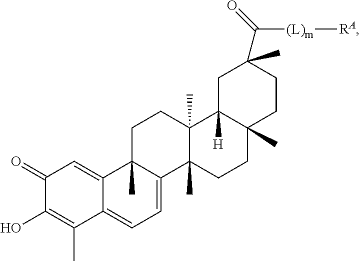

or a pharmaceutically acceptable salt thereof.

In some embodiments, the compound of Formula (I) has formula:

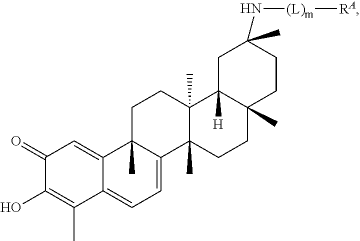

or a pharmaceutically acceptable salt thereof.

In some embodiments, m is an integer from 2 to 20. In some embodiments, m is an integer from 2 to 10. In some embodiments, m is 2, 4, 5, 6, or 8.

In some embodiments, each L is independently selected from N(R^{N}), O, C(═O), C₁₋₆ alkylene, C₃₋₇ cycloalkylene, 4-10-membered heterocycloalkylene, 5-10-membered heteroarylene, C₆₋₁₀ arylene, —(OCH₂CH₂)ₓ—, and —(CH₂CH₂O)ₓ—.

In some embodiments, each L is independently selected from NH, O, C(═O), C₁₋₆ alkylene, C₃₋₇ cycloalkylene, 4-10-membered heterocycloalkylene, 5-10-membered heteroarylene, C₆₋₁₀ arylene, —(OCH₂CH₂)ₓ—, and —(CH₂CH₂O)ₓ—. In some embodiments, at least one L is C₁₋₆ alkylene. In some embodiments, at least one Lis C(═O). In some embodiments, at least one Lis O. In some embodiments, group (L)ₘ comprises at least one 4-10-membered heterocycloalkylene. In some embodiments, group (L)ₘ comprises at least one C₆₋₁₀ arylene. In some embodiments, group (L)ₘ comprises at least one moiety C(═O)O, OC(═O), C(═O)NH, C(═O)NH, NHC(═O)NH, NHC(═S)NH, OC(═O)NH, or NHC(═O)O. In some embodiments, at least one Lis —(OCH₂CH₂)ₓ— or —(CH₂CH₂O)ₓ—.

In some embodiments:
m is 2, 4, 5, 6, or 8;
each L is independently selected from NH, O, C(═O), C₁₋₆ alkylene, C₃₋₇ cycloalkylene, 4-10-membered heterocycloalkylene, 5-10-membered heteroarylene, C₆₋₁₀ arylene, —(OCH₂CH₂)ₓ—, and —(CH₂CH₂O)ₓ—; and
is an integer from 1 to 10.

In some embodiments:
at least one L is C₁₋₆ alkylene;
at least one L is C(═O)O; or
at least one Lis —(OCH₂CH₂)ₓ— or —(CH₂CH₂O)ₓ—; and
is an integer from 1 to 10.

In some embodiments:
(L)ₘ comprises at least one 4-10-membered heterocycloalkylene; or
(L)ₘ comprises at least one C₆₋₁₀ arylene; (L)ₘ comprises at least one moiety C(═O)O, OC(═O), C(═O)NH, C(═O)NH, NHC(═O)NH, NHC(═S)NH, OC(═O)NH, or NHC(═O)O; and
is an integer from 1 to 10.

In some embodiments, x is an integer from 1 to 100. In some embodiments, x is an integer from 1 to 20. In some embodiments, x is an integer from 1 to 10. In some embodiments, x is 1. In some embodiments, x is 2. In some embodiments, x is 3. In some embodiments, x is 1, 2, 3, 4, 5, 6, 7, 8, 9, or 10.

In some embodiments, R^{N} is selected from H and C₁₋₆ alkyl. In some embodiments, R^{N} is H. In some embodiments, R^{N} is C₁₋₆ alkyl. In some embodiments, at least one L is NH. In some embodiments, N(R^{N}) is NH.

In some embodiments, the moiety (L)ₘ is a linker between the R^{A} targeting ligand (i) capable of selectively binding a protein for degradation and the celastrol-containing ubiquitin ligase recruiting ligand (ii). The linker moiety can be flexible or rigid, hydrophobic, hydrophilic, or amphiphilic. In some embodiments, length of the linker moiety is between about 10 Å to about 1,000 Å, from about 15 Å to about 800 Å, from about 20 Å to about 500 Å, from about 20 Å to about 400 Å, from about 20 Å to about 250 Å, from about 20 Å to about 20 Å to about 200 Å, or from about 20 Å to about 150 Å. Without being bound by any theory, it is believed that the length and physical properties of the linker moiety are selected for each targeting ligand and subsequently for each target protein such that upon binding of the celastrol-containing moiety of the E3 ligase, the target protein can be efficiently ubiquitinated within the ligase complex and then degraded by the proteasome machinery of the cell.

In some embodiments, the (L)ₘ moiety comprises any one of the foregoing flexible structural fragments, or any combination thereof:

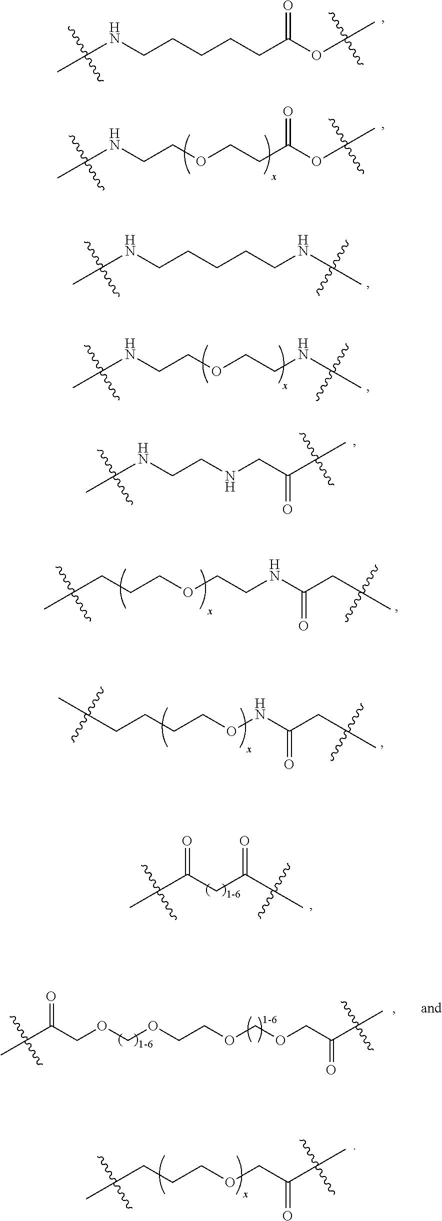

In some embodiments, the (L)ₘ moiety comprises any one of the foregoing rigid structural fragments, or any combination thereof:

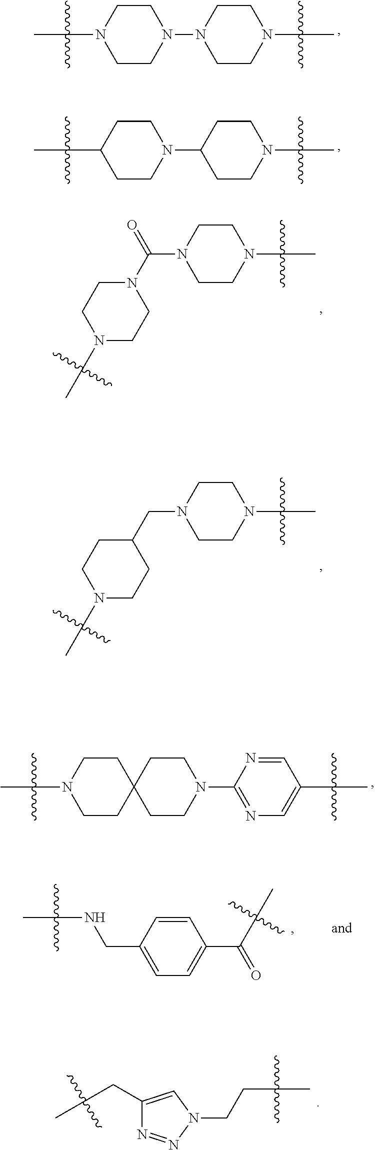

In some embodiments, R^{A} is H. In some embodiments, R^{A} is a targeting ligand capable of selectively binding to a protein (e.g., a pharmacologically relevant protein, such as a protein implicated in a disease or condition). Suitable examples of such proteins include damaged proteins, proteins containing a genetic mutation, aggregation-prone proteins, and other proteins malfunction or excessive functions of which substantially contributes to a pathology of a disease.

### Cancer-Specific Targeting Ligands

In some embodiments, the protein is implicated in the pathology of cancer. Suitable example of such proteins include kinases (cytosolic and receptor), transcription factors, epigenetic writers (e.g., methyltransferases, acetyltransferases,) epigenetic readers, and epigenetic erasers (e.g., demethylases, deacetylases). Examples of methyltransferases include those described in Nature Structural & Molecular Biology volume 26, pages 880-889 (2019), which is incorporated herein by reference in its entirety. Examples of histone demethylases include those described in Nature Reviews Molecular Cell Biology volume 13, pages 297-311 (2012), which is incorporated herein by reference in its entirety. More specifically, suitable examples of such proteins include hormone receptor, androgen receptor (AR), estrogen receptor (ER), estrogen-related receptor alpha (ERRα), KRAS, BRD4 (bromodomain and extraterminal (BET) domain epigenetic reader protein BRD4), BRD2, BRD3, anaplastic lymphoma kinase (ALK), BCL2, BCL6, BCR-ABL, BRD9, BRD7, BTK, CDK4/6, cyclin-dependent kinase 8 (CDK8), cyclin-dependent kinase 9 (CDK9), casein kinase 2 (CK2), c-Met, dihydroorotate dehydrogenase (DHODH), epidermal growth factor receptor (EGFR), human epidermal growth factor receptor 2 (HER2), eukaryotic translation initiation factor 4E (eIF4E), ERK1, ERK2, focal adhesion kinase (FAK), FMS-like tyrosine kinase 3 (FLT3), myeloid cell leukemia 1 (MCL1), murine double minute 2 (MDM2), poly (ADP-ribose) polymerase (PARPs, such as PARP1), transforming acidic coiled-coil containing protein 3 (TACC3), pirin, phosphoinositide 3-kinases (P13Ks), polycomb repressive complex 2 (PRC2), serine-threonine kinase (RIPK2), rpn13, serum/glucocorticoid-inducible protein kinase (SGK), smad3, STAT protein (STAT1, STAT2, STAT3, STAT4, STAT5A, STAT5B, or STAT6), TANK-binding kinase 1 (TBK1), TRIM24, the hepatitis C virus (HCV) NS3 protein, interleukin-1 receptor-associated kinase 4 (IRAK4), P300/CBP-associated factor (PCAF), cellular retinoic acid-binding protein (CRABP-I, -II), anaplastic lymphoma kinase (ALK), mitogen-activated protein kinase 14 (MAPK14, p38-α), mitogen-activated protein kinase 13 (MAPK 13, also known as stress-activated protein kinase 4 (SAPK4), or p38-8), sirtuin, sirtuin2 (SIRT2), P300/CBP associating factor (PCAF), histone deacetylase (e.g., HDAC1, HDAC2, HDAC3, HDAC4, HDAC5, HDAC6,m HDAC7, HDAC8, HDAC9, HDAC10, or HDAC11), cytosolic aminoacyl tRNA synthetase, mitochondrial aminoacyl tRNA synthetase, PD-L1, CD47, cytokine (e.g., IL-2, IL-7, IL-12, IL-15, IL-10, IL-21, or INF-alfa), chemokine (e.g., CCL2, CCL3, or CCL5), and an immunosuppressive antigen (e.g., PD-1, CTLA-4, CD20, Lag-3 or Tim-3).

Suitable examples of targeting ligands R^{A} capable of binding to the proteins implicated in the pathology of cancer include peptides and small-molecule therapeutics. Generally, small-molecule therapeutics are low molecular weight organic compounds, typically about 2000 daltons (Da) or less. The small-molecule therapeutics may include various heterocyclic moieties, such as pyridine, pyrimidine, pyrazole, oxazole, or indole, and various functional groups, such as hydroxy, carboxy, or amino groups. The small-molecule targeting ligand R^{A} may bind target proteins covalently or non-covalently, reversibly or irreversibly. Examples of non-covalent binding include electrostatic interactions, hydrophobic interactions, and Van der Waals forces. Examples of covalent binding include chemical reactions with functional groups within side-chains of amino acids target proteins, such as cysteine and lysine. In some embodiments, the targeting ligand binds the protein selectively, that is, the ligand does not substantially bind to any other protein in the cell. For example, the targeting ligand R^{A} is about 10×, about 20×, about 50×, about 100×, or about 1,000× selective to the target protein compared to any other protein in the cell, or any combination thereof. Examples of small-molecule targeting ligands R^{A} capable of selective binding to the target proteins implicated in the pathology of cancer include JQ1 VZ185, imatinib, enzalutamide, fulvestrant, tazemetostat, MAK683, UNC1999, adavosertib, AZD1775, carfilzomib, MG-132, apigenin, alectinib, brigatinib, ceritinib, crizotinib, and lorlatinib, TAE684, bosutinib, dasatinib, SNS-032, CX-4945, foretinib, OTX-15, brequinar, lapatinib, gefitinib, afatinib, fulvestrant, defactinib, quizartinib, gilteritinib, MLN-518, sunitinib, ponatinib, MI-1061, olaparib, niraparib, iniparib, veliparib, and bortezomib.

### Neurodegeneration-Specific Targeting Ligands

In some embodiments, the protein is implicated in the pathology of a neurodegenerative disease or condition. Suitable examples of such proteins include alpha-synuclein, transthyretin, tau protein, and amyloid-β peptide. Suitable examples of targeting ligands R^{A} capable of binding to the proteins implicated in the pathology of neurodegenerative diseases include peptides and small-molecule therapeutics. More specifically, suitable examples of such R^{A} groups include KLVFF and curcumin.

In some embodiments, the compound of Formula (I) is selected from any one of the following compounds:

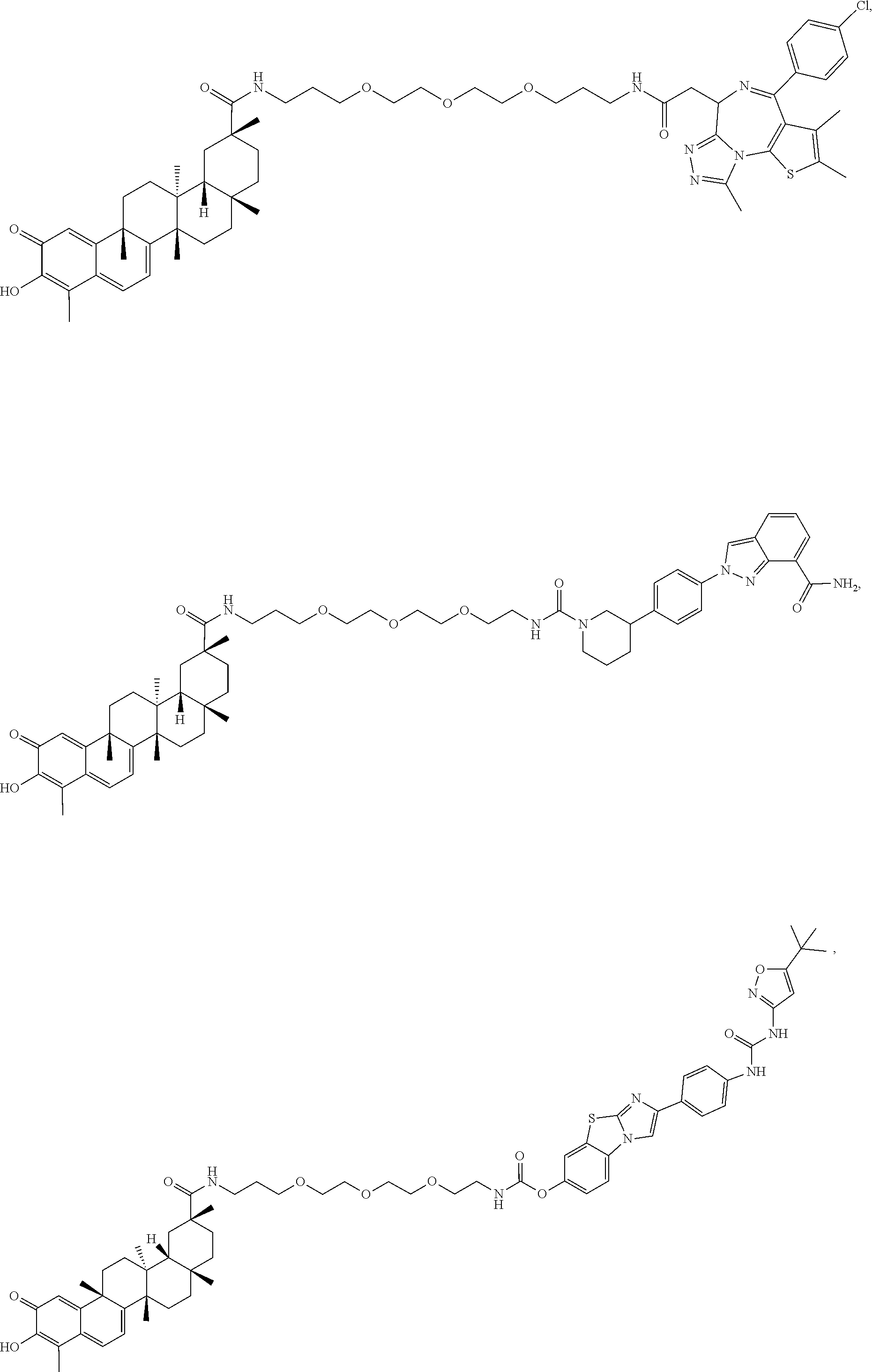

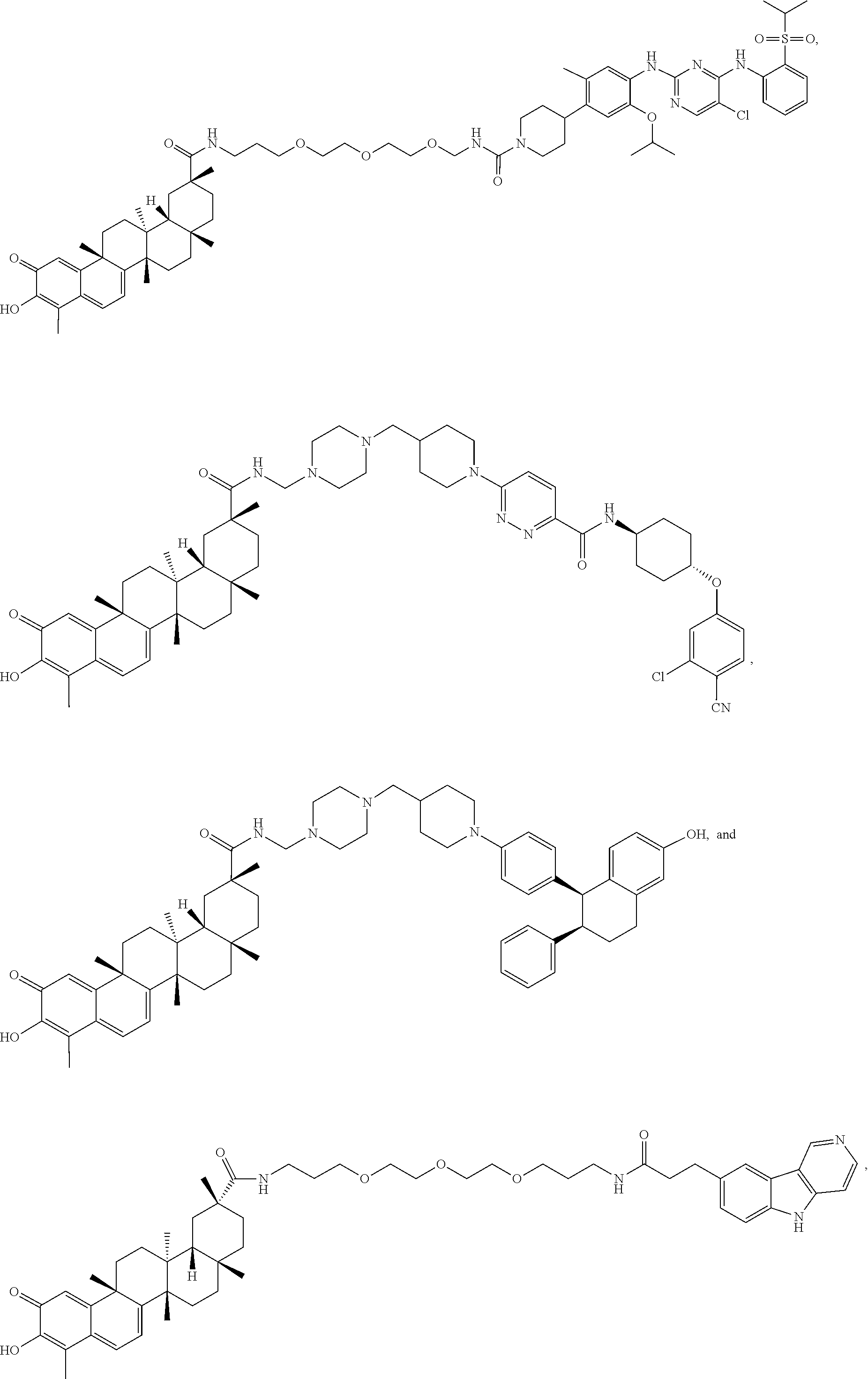

or a pharmaceutically acceptable salt thereof.

### Pharmaceutically Acceptable Salts

In some embodiments, a salt of any one of the compounds of the present disclosure is formed between an acid and a basic group of the compound, such as an amino functional group, or a base and an acidic group of the compound, such as a carboxyl functional group. According to another embodiment, the compound is a pharmaceutically acceptable acid addition salt. In some embodiments, acids commonly employed to form pharmaceutically acceptable salts of the compounds include inorganic acids such as hydrogen bisulfide, hydrochloric acid, hydrobromic acid, hydroiodic acid, sulfuric acid and phosphoric acid, as well as organic acids such as para-toluenesulfonic acid, salicylic acid, tartaric acid, bitartaric acid, ascorbic acid, maleic acid, besylic acid, fumaric acid, gluconic acid, glucuronic acid, formic acid, glutamic acid, methanesulfonic acid, ethanesulfonic acid, benzenesulfonic acid, lactic acid, oxalic acid, para-bromophenylsulfonic acid, carbonic acid, succinic acid, citric acid, benzoic acid and acetic acid, as well as related inorganic and organic acids. Such pharmaceutically acceptable salts thus include sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate, caprylate, acrylate, formate, isobutyrate, caprate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, terephthalate, sulfonate, xylene sulfonate, phenylacetate, phenylpropionate, phenylbutyrate, citrate, lactate, β-hydroxybutyrate, glycolate, maleate, tartrate, methanesulfonate, propanesulfonate, naphthalene-1-sulfonate, naphthalene-2-sulfonate, mandelate and other salts. In one embodiment, pharmaceutically acceptable acid addition salts include those formed with mineral acids such as hydrochloric acid and hydrobromic acid, and especially those formed with organic acids such as maleic acid.

In some embodiments, bases commonly employed to form pharmaceutically acceptable salts of the compounds include hydroxides of alkali metals, including sodium, potassium, and lithium; hydroxides of alkaline earth metals such as calcium and magnesium; hydroxides of other metals, such as aluminum and zinc; ammonia, organic amines such as unsubstituted or hydroxyl-substituted mono-, di-, or tri-alkylamines, dicyclohexylamine; tributyl amine; pyridine; N-methyl, N-ethylamine; diethylamine; triethylamine; mono-, bis-, or tris-(2-OH—(C₁-C₆)-alkylamine), such as N,N-dimethyl-N-(2-hydroxyethyl)amine or tri-(2-hydroxyethyl)amine; N-methyl-D-glucamine; morpholine; thiomorpholine; piperidine; pyrrolidine; and amino acids such as arginine, lysine, and the like.

### Methods of Use

Without being bound by any particular theory, it is believed that the binding of the celastrol-containing moiety of the PROTACs of this disclosure to a ligase leads to ubiquitination and subsequent degradation of the ligase complex. When a protein target is bound to the targeting ligand (i) of the PROTAC molecule, a complex is formed between the ligase and the protein target. As a result, the protein target is ubiquitinated within the ligase complex and is further degraded by the proteasome machinery of the cell.

Hence, when the protein target is implicated in a pathology of a disease or condition, contacting a cell containing the target protein by a celastrol moiety-containing PROTAC of this disclosure results in degradation of the target protein and, concomitantly, a favorable therapeutic outcome.

Accordingly, in some embodiments, the present disclosure provides a method of reducing level of a protein (e.g., a target protein) in a cell, the method comprising contacting the cell with an effective amount of a compound of Formula (I), or a pharmaceutically acceptable salt thereof. Contacting the cell may occur in vitro, in vivo, or ex vivo.

In some embodiments, when a target protein to which targeting ligand R^{A} within Formula (I) can bind is implicated in the pathology of cancer, the present disclosure provides a method of treating cancer, the method comprising administering to a patient in need thereof a therapeutically effective amount of a compound of Formula (I), or a pharmaceutically acceptable salt thereof. Suitable examples of cancers include bladder cancer, brain cancer, breast cancer, colorectal cancer, cervical cancer, gastrointestinal cancer, genitourinary cancer, head and neck cancer, lung cancer, ovarian cancer, pancreatic cancer, prostate cancer, renal cancer, skin cancer, and testicular cancer. In some embodiments, the cancer is selected from sarcoma, angiosarcoma, fibrosarcoma, rhabdomyosarcoma, liposarcoma, myxoma, rhabdomyoma, fibroma, lipoma, teratoma, lung cancer, bronchogenic carcinoma squamous cell, undifferentiated small cell, undifferentiated large cell, adenocarcinoma, alveolar bronchiolar carcinoma, bronchial adenoma, sarcoma, lymphoma, chondromatous hamartoma, mesothelioma, gastrointestinal cancer, cancer of the esophagus, squamous cell carcinoma, adenocarcinoma, leiomyosarcoma, lymphoma, cancer of the stomach, carcinoma, lymphoma, leiomyosarcoma, cancer of the pancreas, ductal adenocarcinoma, insulinoma, glucagonoma, gastrinoma, carcinoid tumor, vipoma, cancer of the small bowel, adenocarcinoma, lymphoma, carcinoid tumors, Kaposi's sarcoma, leiomyoma, hemangioma, lipoma, neurofibroma, fibroma, cancer of the large bowel or colon, tubular adenoma, villous adenoma, hamartoma, leiomyoma, genitourinary tract cancer, cancer of the kidney adenocarcinoma, Wilm's tumor (nephroblastoma), cancer of the bladder, cancer of the urethra, squamous cell carcinoma, transitional cell carcinoma, cancer of the prostate, cancer of the testis, seminoma, teratoma, embryonal carcinoma, teratocarcinoma, choriocarcinoma, sarcoma, interstitial cell carcinoma, fibroma, fibroadenoma, adenomatoid tumors, lipoma, liver cancer, hepatoma hepatocellular carcinoma, cholangiocarcinoma, hepatoblastoma, angiosarcoma, hepatocellular adenoma, hemangioma, bone cancer, osteogenic sarcoma (osteosarcoma), fibrosarcoma, malignant fibrous histiocytoma, chondrosarcoma, Ewing's sarcoma, malignant lymphoma (reticulum cell sarcoma), malignant giant cell tumor, chordoma, osteochrondroma (osteocartilaginous exostoses), benign chondroma, chondroblastoma, chondromyxofibroma, osteoid osteoma giant cell tumor, nervous system cancer, cancer of the skull, osteoma, hemangioma, granuloma, xanthoma, osteitis deformans, cancer of the meninges meningioma, meningiosarcoma, gliomatosis, cancer of the brain, astrocytoma, medulloblastoma, glioma, ependymoma, germinoma (pinealoma), glioblastoma multiforme, oligodendroglioma, schwannoma, retinoblastoma, congenital tumors, cancer of the spinal cord, neurofibroma, meningioma, glioma, sarcoma, gynecological cancer, cancer of the uterus, endometrial carcinoma, cancer of the cervix, cervical carcinoma, pre tumor cervical dysplasia, cancer of the ovaries, ovarian carcinoma, serous cystadenocarcinoma, mucinous cystadenocarcinoma, unclassified carcinoma, granulosa-theca cell tumor, Sertoli Leydig cell tumor, dysgerminoma, malignant teratoma, cancer of the vulva, squamous cell carcinoma, intraepithelial carcinoma, adenocarcinoma, fibrosarcoma, melanoma, cancer of the vagina, clear cell carcinoma, squamous cell carcinoma, botryoid sarcoma, embryonal rhabdomyosarcoma, cancer of the fallopian tubes, hematologic cancer, cancer of the blood, lymphoma, leukemia, acute myeloid leukemia (AML), chronic myeloid leukemia (CML), acute lymphoblastic leukemia (ALL), chronic lymphoblastic leukemia, chronic lymphocytic leukemia, myeloproliferative diseases, multiple myeloma, myelodysplastic syndrome, Hodgkin's lymphoma, non-Hodgkin's lymphoma (malignant lymphoma), Waldenstrom's macroglobulinemia, skin cancer, malignant melanoma, basal cell carcinoma, squamous cell carcinoma, Kaposi's sarcoma, moles dysplastic nevi, lipoma, angioma, dermatofibroma, keloids, adrenal gland cancer, and neuroblastoma. In some embodiments, the cancer is brain cancer or breast cancer.

In some embodiments, the disease is cancer selected from midline carcinoma, multiple myeloma, acute myelogenous leukemia, and acute lymphoblastic leukemia, the protein implicated in the disease is BRD4, and the compound of Formula (I) is:

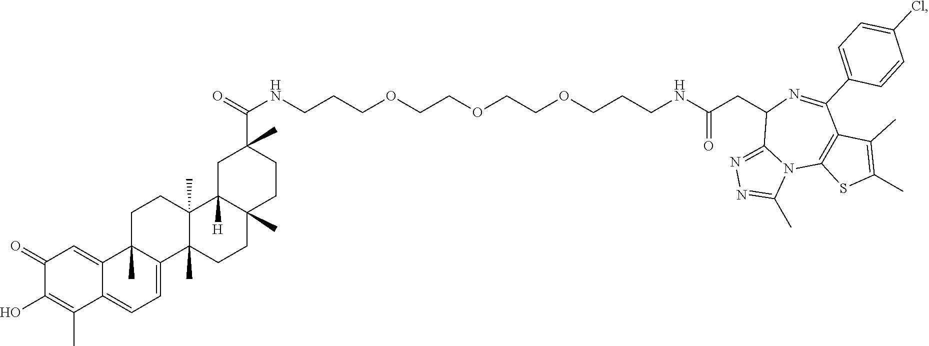

or a pharmaceutically acceptable salt thereof.

In some embodiments, the disease is cancer selected from ovarian cancer, fallopian tube cancer, and peritoneal cancer, the protein implicated in the disease is PARP, and the compound of Formula (I) is:

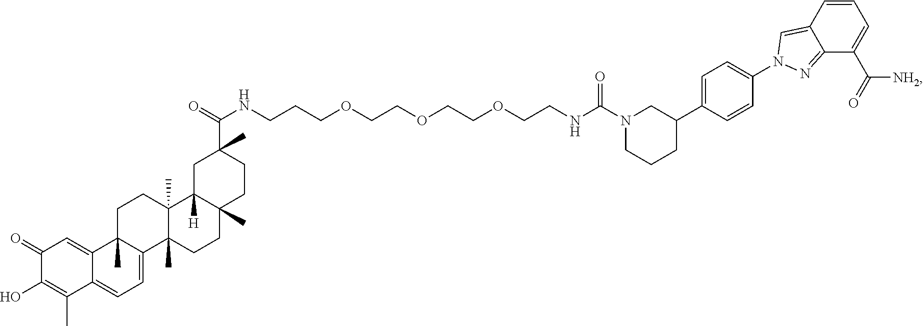

or a pharmaceutically acceptable salt thereof.

In some embodiments, the disease is acute myeloid leukemia, the protein implicated in the disease is receptor tyrosine kinase FLT3, and the compound of Formula (I) is:

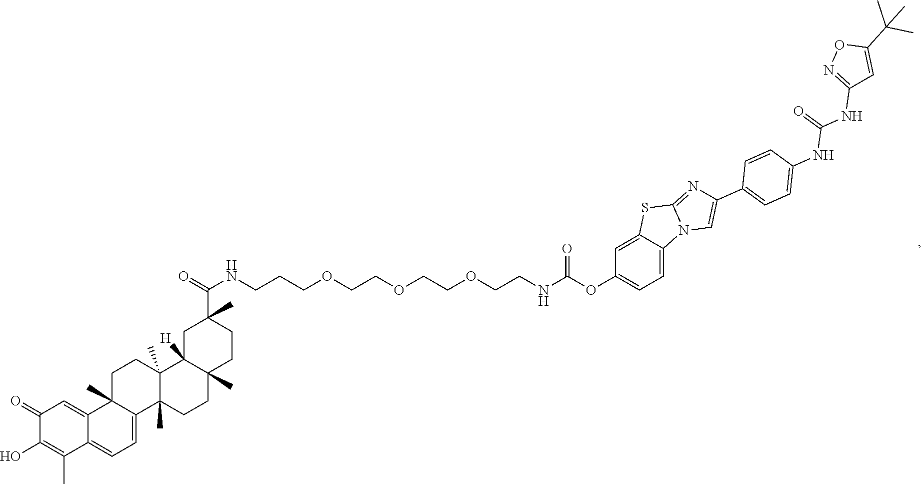 [SMILES: CC1=C(O)C(=O)C=C2C1=CC=C1[C@@]2(C)CC[C@@]2(C)[C@@H]3C[C@](C)(C(=O)NCCCOCCOCCOCCNC(=O)Oc4ccc5c(c4)sc4nc(-c6ccc(NC(=O)Nc7cc(C(C)(C)C)on7)cc6)cn45)CC[C@]3(C)CC[C@]12C]

or a pharmaceutically acceptable salt thereof.

In some embodiments, the disease is non-small cell lung cancer, the protein implicated in the disease is ALK, and the compound of Formula (I) is:

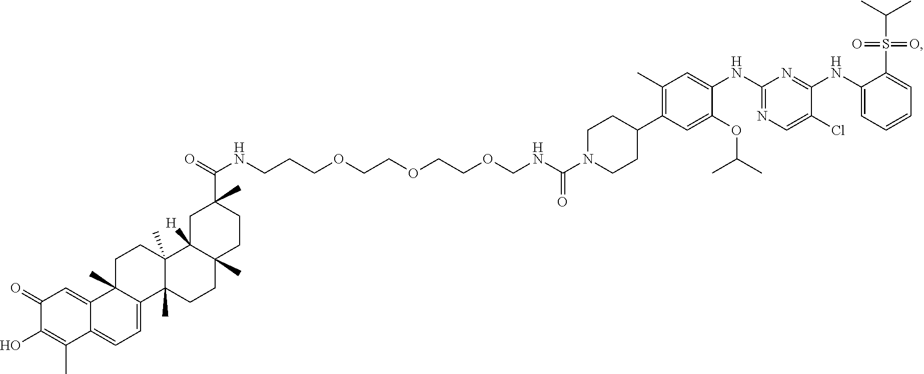 [SMILES: CC1=C(O)C(=O)C=C2C1=CC=C1[C@@]2(C)CC[C@@]2(C)[C@@H]3C[C@](C)(C(=O)NCCCOCCOCCOCNC(=O)N4CCC(c5cc(OC(C)C)c(Nc6ncc(Cl)c(Nc7ccccc7S(=O)(=O)C(C)C)n6)cc5C)CC4)CC[C@]3(C)CC[C@]12C]

or a pharmaceutically acceptable salt thereof.

In some embodiments, the disease is prostate cancer, the protein implicated in the disease is AR, and the compound of Formula (I) is:

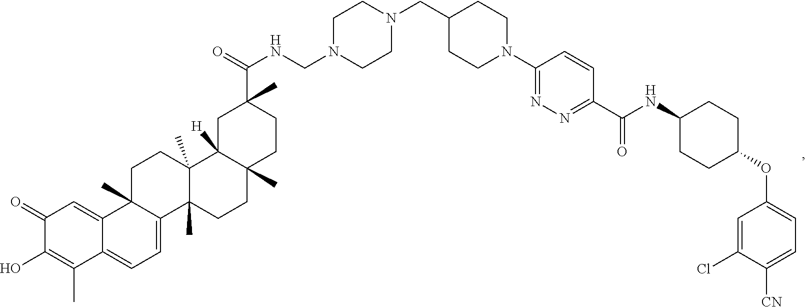 [SMILES: CC1=C(O)C(=O)C=C2C1=CC=C1[C@@]2(C)CC[C@@]2(C)[C@@H]3C[C@](C)(C(=O)NCN4CCN(CC5CCN(c6ccc(C(=O)N[C@H]7CC[C@H](Oc8ccc(C#N)c(Cl)c8)CC7)nn6)CC5)CC4)CC[C@]3(C)CC[C@]12C]

or a pharmaceutically acceptable salt thereof.

In some embodiments, the disease is breast cancer, the protein implicated in the disease is ER, and the compound of Formula (I) is cmpd 1.

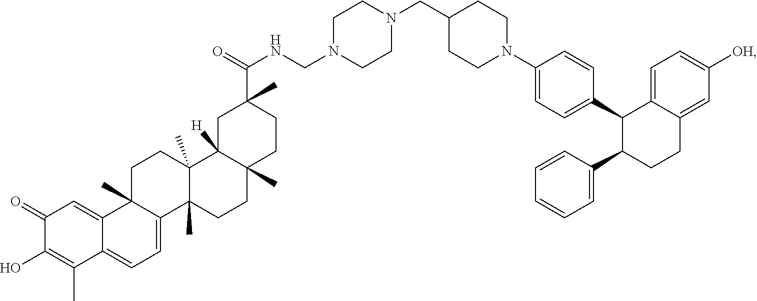 [SMILES: CC1=C(O)C(=O)C=C2C1=CC=C1[C@@]2(C)CC[C@@]2(C)[C@@H]3C[C@](C)(C(=O)NCN4CCN(CC5CCN(c6ccc([C@H]7c8ccc(O)cc8CC[C@H]7c7ccccc7)cc6)CC5)CC4)CC[C@]3(C)CC[C@]12C]

or a pharmaceutically acceptable salt thereof.

In some embodiments, when a target protein to which targeting ligand R^{A} within Formula (I) can bind is implicated in the pathology of is an inflammatory disease or condition, the present disclosure provides a method of treating is an inflammatory disease or condition, the method comprising administering to a patient in need thereof a therapeutically effective amount of a compound of Formula (I), or a pharmaceutically acceptable salt thereof. Suitable examples of such conditions include psoriasis, gastritis, colitis, arthritis, pneumonia, liver damage, cardiovascular disease, chronic obstructive pulmonary disease, emphysema, lung injury, arthritis, autoimmune disease, sepsis, fibrosis, diabetes, and stroke.

In some embodiments, when a target protein to which targeting ligand R^{A} within Formula (I) can bind is implicated in the pathology of is a neurodegenerative disease or condition, the present disclosure provides a method of treating is a neurodegenerative disease or condition, the method comprising administering to a patient in need thereof a therapeutically effective amount of a compound of Formula (I), or a pharmaceutically acceptable salt thereof. Suitable examples of neurodegenerative diseases include Huntington's disease (HD), Alzheimer's disease (AD), Parkinson's disease (PD), multiple sclerosis (MS), frontotemporal dementia (FTD) and amyotrophic lateral sclerosis (ALS). Other examples of neurodegenerative disease include Lewy body disease, dementia, motor neuron disease (MND), Prion disease, cerebral amyloid angiopathy, vascular cognitive impairment (VCI), hippocampal sclerosis, Binswanger's disease, Creutzfeldt-Jakob disease, cerebral ischemia, cerebrovascular ischemia, brain ischemia, cerebral palsy, chemotherapy-induced brain damage; cisplatin-induced neurotoxicity, diabetic neuropathy; Down's syndrome, epilepsy and post-traumatic epilepsy; Friedreich's ataxia; Hallervorden-Spatz disease; macular degeneration; methanol-induced neurotoxicity; meningitis (aseptic and tuberculous); Pick's disease; progressive supra-nuclear palsy; radiotherapy-induced brain damage; senile dementia; schizophrenia; traumatic brain injury (TBI); traumatic spinal injury; viral meningitis; encephalitis, and viral encephalitis.

### Compositions, Formulations, and Routes of Administration

The present application also provides pharmaceutical compositions comprising an effective amount of a compound of the present disclosure (e.g., Formula (I)) disclosed herein, or a pharmaceutically acceptable salt thereof; and a pharmaceutically acceptable carrier. The pharmaceutical composition may also comprise any one of the additional therapeutic agents described herein. In certain embodiments, the application also provides pharmaceutical compositions and dosage forms comprising any one the additional therapeutic agents described herein. The carrier(s) are “acceptable” in the sense of being compatible with the other ingredients of the formulation and, in the case of a pharmaceutically acceptable carrier, not deleterious to the recipient thereof in an amount used in the medicament.

Pharmaceutically acceptable carriers, adjuvants and vehicles that may be used in the pharmaceutical compositions of the present application include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol, and wool fat.

The compositions or dosage forms may contain any one of the compounds and therapeutic agents described herein in the range of 0.005% to 100% with the balance made up from the suitable pharmaceutically acceptable excipients. The contemplated compositions may contain 0.001%-100% of any one of the compounds and therapeutic agents provided herein, in one embodiment 0.1-95%, in another embodiment 75-85%, in a further embodiment 20-80%, wherein the balance may be made up of any pharmaceutically acceptable excipient described herein, or any combination of these excipients.

### Routes of Administration and Dosage Forms

The pharmaceutical compositions of the present application include those suitable for any acceptable route of administration. Acceptable routes of administration include, but are not limited to, buccal, cutaneous, endocervical, endosinusial, endotracheal, enteral, epidural, interstitial, intra-abdominal, intra-arterial, intrabronchial, intrabursal, intracerebral, intracisternal, intracoronary, intradermal, intraductal, intraduodenal, intradural, intraepidermal, intraesophageal, intragastric, intragingival, intraileal, intralymphatic, intramedullary, intrameningeal, intramuscular, intranasal, intraovarian, intraperitoneal, intraprostatic, intrapulmonary, intrasinal, intraspinal, intrasynovial, intratesticular, intrathecal, intratubular, intratumoral, intrauterine, intravascular, intravenous, nasal, nasogastric, oral, parenteral, percutaneous, peridural, rectal, respiratory (inhalation), subcutaneous, sublingual, submucosal, topical, transdermal, transmucosal, transtracheal, ureteral, urethral and vaginal.

Compositions and formulations described herein may conveniently be presented in a unit dosage form, e.g., tablets, sustained release capsules, and in liposomes, and may be prepared by any methods well known in the art of pharmacy. See, for example, Remington: The Science and Practice of Pharmacy, Lippincott Williams & Wilkins, Baltimore, MD (20th ed. 2000). Such preparative methods include the step of bringing into association with the molecule to be administered ingredients such as the carrier that constitutes one or more accessory ingredients. In general, the compositions are prepared by uniformly and intimately bringing into association the active ingredients with liquid carriers, liposomes or finely divided solid carriers, or both, and then, if necessary, shaping the product.

In some embodiments, any one of the compounds and therapeutic agents disclosed herein are administered orally. Compositions of the present application suitable for oral administration may be presented as discrete units such as capsules, sachets, granules or tablets each containing a predetermined amount (e.g., effective amount) of the active ingredient; a powder or granules; a solution or a suspension in an aqueous liquid or a non-aqueous liquid; an oil-in-water liquid emulsion; a water-in-oil liquid emulsion; packed in liposomes; or as a bolus, etc. Soft gelatin capsules can be useful for containing such suspensions, which may beneficially increase the rate of compound absorption. In the case of tablets for oral use, carriers that are commonly used include lactose, sucrose, glucose, mannitol, and silicic acid and starches. Other acceptable excipients may include: a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. For oral administration in a capsule form, useful diluents include lactose and dried corn starch. When aqueous suspensions are administered orally, the active ingredient is combined with emulsifying and suspending agents. If desired, certain sweetening and/or flavoring and/or coloring agents may be added. Compositions suitable for oral administration include lozenges comprising the ingredients in a flavored basis, usually sucrose and acacia or tragacanth; and pastilles comprising the active ingredient in an inert basis such as gelatin and glycerin, or sucrose and acacia.

Compositions suitable for parenteral administration include aqueous and non-aqueous sterile injection solutions or infusion solutions which may contain antioxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example, sealed ampules and vials, and may be stored in a freeze dried (lyophilized) condition requiring only the addition of the sterile liquid carrier, for example water for injections, saline (e.g., 0.9% saline solution) or 5% dextrose solution, immediately prior to use. Extemporaneous injection solutions and suspensions may be prepared from sterile powders, granules and tablets. The injection solutions may be in the form, for example, of a sterile injectable aqueous or oleaginous suspension. This suspension may be formulated according to techniques known in the art using suitable dispersing or wetting agents and suspending agents. The sterile injectable preparation may also be a sterile injectable solution or suspension in a non-toxic parenterally-acceptable diluent or solvent, for example, as a solution in 1,3-butanediol. Among the acceptable vehicles and solvents that may be employed are mannitol, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant.

The pharmaceutical compositions of the present application may be administered in the form of suppositories for rectal administration. These compositions can be prepared by mixing a compound of the present application with a suitable non-irritating excipient which is solid at room temperature but liquid at the rectal temperature and therefore will melt in the rectum to release the active components. Such materials include, but are not limited to, cocoa butter, beeswax, and polyethylene glycols.

The pharmaceutical compositions of the present application may be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art. See, for example, U.S. Pat. No. 6,803,031. Additional formulations and methods for intranasal administration are found in Ilium, L., J Pharm Pharmacol, 56:3-17, 2004 and Ilium, L., Eur J Pharm Sci 11:1-18, 2000.

The topical compositions of the present disclosure can be prepared and used in the form of an aerosol spray, cream, emulsion, solid, liquid, dispersion, foam, oil, gel, hydrogel, lotion, mousse, ointment, powder, patch, pomade, solution, pump spray, stick, towelette, soap, or other forms commonly employed in the art of topical administration and/or cosmetic and skin care formulation. The topical compositions can be in an emulsion form. Topical administration of the pharmaceutical compositions of the present application is especially useful when the desired treatment involves areas or organs readily accessible by topical application. In some embodiments, the topical composition comprises a combination of any one of the compounds and therapeutic agents disclosed herein, and one or more additional ingredients, carriers, excipients, or diluents including, but not limited to, absorbents, anti-irritants, anti-acne agents, preservatives, antioxidants, coloring agents/pigments, emollients (moisturizers), emulsifiers, film-forming/holding agents, fragrances, leave-on exfoliants, prescription drugs, preservatives, scrub agents, silicones, skin-identical/repairing agents, slip agents, sunscreen actives, surfactants/detergent cleansing agents, penetration enhancers, and thickeners.

The compounds and therapeutic agents of the present application may be incorporated into compositions for coating an implantable medical device, such as prostheses, artificial valves, vascular grafts, stents, or catheters. Suitable coatings and the general preparation of coated implantable devices are known in the art and are exemplified in U.S. Pat. Nos. 6,099,562; 5,886,026; and 5,304, 121. The coatings are typically biocompatible polymeric materials such as a hydrogel polymer, polymethyldisiloxane, polycaprolactone, polyethylene glycol, polylactic acid, ethylene vinyl acetate, and mixtures thereof. The coatings may optionally be further covered by a suitable topcoat of fluorosilicone, polysaccharides, polyethylene glycol, phospholipids or combinations thereof to impart controlled release characteristics in the composition. Coatings for invasive devices are to be included within the definition of pharmaceutically acceptable carrier, adjuvant or vehicle, as those terms are used herein.

According to another embodiment, the present application provides an implantable drug release device impregnated with or containing a compound or a therapeutic agent, or a composition comprising a compound of the present application or a therapeutic agent, such that said compound or therapeutic agent is released from said device and is therapeutically active.

### Dosages and Regimens

In the pharmaceutical compositions of the present application, a compound of the present disclosure (e.g., a compound of Formula (I)) is present in an effective amount (e.g., a therapeutically effective amount). Effective doses may vary, depending on the diseases treated, the severity of the disease, the route of administration, the sex, age and general health condition of the subject, excipient usage, the possibility of co-usage with other therapeutic treatments such as use of other agents and the judgment of the treating physician.

In some embodiments, an effective amount of the compound (e.g., Formula (I)) can range, for example, from about 0.001 mg/kg to about 500 mg/kg (e.g., from about 0.001 mg/kg to about 200 mg/kg; from about 0.01 mg/kg to about 200 mg/kg; from about 0.01 mg/kg to about 150 mg/kg; from about 0.01 mg/kg to about 100 mg/kg; from about 0.01 mg/kg to about 50 mg/kg; from about 0.01 mg/kg to about 10 mg/kg; from about 0.01 mg/kg to about 5 mg/kg; from about 0.01 mg/kg to about 1 mg/kg; from about 0.01 mg/kg to about 0.5 mg/kg; from about 0.01 mg/kg to about 0.1 mg/kg; from about 0.1 mg/kg to about 200 mg/kg; from about 0.1 mg/kg to about 150 mg/kg; from about 0.1 mg/kg to about 100 mg/kg; from about 0.1 mg/kg to about 50 mg/kg; from about 0.1 mg/kg to about 10 mg/kg; from about 0.1 mg/kg to about 5 mg/kg; from about 0.1 mg/kg to about 2 mg/kg; from about 0.1 mg/kg to about 1 mg/kg; or from about 0.1 mg/kg to about 0.5 mg/kg). In some embodiments, an effective amount of a compound of Formula (I), or a pharmaceutically acceptable salt thereof, is about 0.1 mg/kg, about 0.5 mg/kg, about 1 mg/kg, about 2 mg/kg, or about 5 mg/kg.

The foregoing dosages can be administered on a daily basis (e.g., as a single dose or as two or more divided doses, e.g., once daily, twice daily, thrice daily) or non-daily basis (e.g., every other day, every two days, every three days, once weekly, twice weekly, once every two weeks, once a month).

### Kits

The present invention also includes pharmaceutical kits useful, for example, in the treatment of disorders, diseases and conditions referred to herein, which include one or more containers containing a pharmaceutical composition comprising a therapeutically effective amount of a compound of the present disclosure. Such kits can further include, if desired, one or more of various conventional pharmaceutical kit components, such as, for example, containers with one or more pharmaceutically acceptable carriers, additional containers, etc. Instructions, either as inserts or as labels, indicating quantities of the components to be administered, guidelines for administration, and/or guidelines for mixing the components, can also be included in the kit. The kit may optionally include an additional therapeutic agent as described herein.

### Combinations

The compounds of the present disclosure can be used on combination with at least one medication or therapy useful, e.g., in treating or alleviating symptoms of cancer, an inflammatory condition, or a neurodegenerative disease or condition. Suitable examples of anticancer agents include cisplatin, carboplatin, oxaliplatin, satraplatin, picoplatin, nedaplatin, triplatin, lipoplatin, nitrogen mustards, cyclophosphamide, mechlorethamine or mustine (HN2), uramustine or uracil mustard, melphalan, chlorambucil, ifosfamide, bendamustine, nitrosoureas, carmustine, lomustine, streptozocin, alkyl sulfonates, busulfan, thiotepa, procarbazine, altretamine, triazenes, dacarbazine, mitozolomide, temozolomide, necitumumab, dinutuximab, nivolumab, blinatumomab, pembrolizumab, ramucirumab, obinutuzumab, adotrastuzumab emtansine, pertuzumab, brentuximab, ipilimumab, ofatumumab, catumaxomab, bevacizumab, cetuximab, tositumomab, ibritumomab tiuxetan, alemtuzumab, gemtuzumab ozogamicin, trastuzumab, rituximab, vinblastine, vincristine, vindesine, vinorelbine, desoxyvincaminol, vincaminol, vinburnine, vincamajine, vineridine, vinburnine, vinpocetine, fluorouracil, cladribine, capecitabine, mercaptopurine, pemetrexed, fludarabine, gemcitabine, hydroxyurea, methotrexate, nelarbine, clofarabine, cytarabine, decitabine, pralatrexate, floxuridine, and thioguanine, or a pharmaceutically acceptable salt thereof. In some embodiments, the anti-cancer therapy is selected from the group consisting of surgery, radiation therapy, chemotherapy, gene therapy, DNA therapy, viral therapy, RNA therapy, adjuvant therapy, and immunotherapy. Suitable examples of anti-cancer agents also include bromodomain inhibitors (e.g., BRD2, BRD3, BRD4, or BRDT inhibitors). Examples of such inhibitors include I-BET 151, JQ1, I-BET 762, OTX-015, TEN-010, CPI-203, CPI-0610, olinone, RVX-208, ABBV-744, LY294002, AZD5153, MT-1, and MS645. Suitable examples of anti-cancer agents also include immunotherapies, such as anti-PD1 antibodies or anti-CTLA4 antibodies.

Suitable examples of anti-inflammatory agents include steroids, antibodies against IL-6 or TNF-alpha, and NSAIDs (e.g., aspirin, acetaminophen, ibuprofen, naproxen, or diclofenac). Suitable examples of therapies for a neurodegenerative disease include L-DOPA, memantine, riluzole, edaravone, tetrabenazine, nicotinamide riboside, and nicotinamide mononucleotide.

The compound of the present disclosure may be administered to the patient simultaneously with the additional therapeutic agent (in the same pharmaceutical composition or dosage form or in different compositions or dosage forms) or consecutively (the additional therapeutic agent may be administered in a separate pharmaceutical composition or dosage form before or after administration of the compound of the present disclosure).

In some embodiments, the present disclosure provides a method of treating cancer (e.g., any one of cancers described herein), the method comprising administering a subject in need thereof:
(i) a therapeutically effective amount of celastrol having formula:

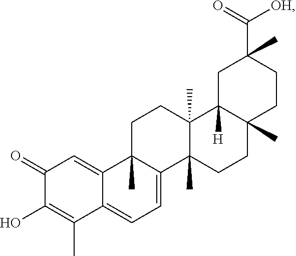

or a pharmaceutically acceptable salt thereof, and
(ii) a therapeutically effective amount of a bromodomain inhibitor, or a pharmaceutically acceptable salt thereof.

In some embodiments, the method includes administering an additional therapeutic agent useful in treating cancer (e.g., any one of the additional chemotherapeutic agents described herein). For example, the method may include administering celastrol and bromodomain inhibitor in combination with cisplatin, carboplatin, oxaliplatin, satraplatin, picoplatin, or an anticancer antibody, such as necitumumab, dinutuximab, nivolumab, blinatumomab, pembrolizumab, ramucirumab, obinutuzumab, adotrastuzumab, pertuzumab, brentuximab, ipilimumab, ofatumumab, catumaxomab, bevacizumab, cetuximab, tositumomab, ibritumomab tiuxetan, alemtuzumab, gemtuzumab ozogamicin, trastuzumab, or rituximab.

In some embodiments, the method includes treating a cancer where bromodomain protein (e.g., BRD4) is implicated in pathology. In one example, the method includes treating a cancer responsive to treatment with a bromodomain inhibitor. Suitable examples of such cancers include the cancers described herein. Some examples of such cancers include glioma (including pediatric glioma), breast cancer (including metastatic breast cancer and triple-negative breast cancer), midline carcinoma, ovarian cancer, prostate cancer (including metastatic and/or castration-resistant), leukemia (including acute myeloid leukemia), lymphoma (including diffuse large B-cell lymphoma), multiple myeloma, non-small-cell lung cancer, glioblastoma multiforme, head and neck cancer, colorectal cancer, gastric cancer, endometrial cancer, cervical cancer, glioblastoma, medulloblastoma, hepatocellular carcinoma, colon cancer, pancreatic cancer, liver cancer, as well as other cancers. In some embodiments, the cancer is selected from breast cancer, glioma, and pediatric glioma.

In some embodiments, the therapeutically effective amount of celastrol and the therapeutically effective amount of bromodomain inhibitor are independently selected from any of the amounts described herein for Formula (I). For example, the therapeutically effective amount of celastrol may range from about 0.001 mg/kg to about 500 mg/kg, and the therapeutically effective amount of bromodomain inhibitor may range from about 0.001 mg/kg to about 500 mg/kg.

In some embodiments, the celastrol and the bromodomain inhibitor can be administered to the subject by any route and in any dosage form as described herein for formula (I). For example, both celastrol and bromodomain inhibitor can be administered to the subject intravenously, both celastrol and bromodomain inhibitor can be administered to the subject orally, or one of the agents can be administered orally and the other agent can be administered intravenously. In some embodiments, the celastrol, or a pharmaceutically acceptable salt thereof, and a bromodomain inhibitor, or a pharmaceutically acceptable salt thereof, are administered to the subject simultaneously. In some embodiments, the celastrol, or a pharmaceutically acceptable salt thereof, and the bromodomain inhibitor, or a pharmaceutically acceptable salt thereof, and administered to the subject consecutively. In some embodiments, the celastrol, or a pharmaceutically acceptable salt thereof, and the bromodomain inhibitor, or a pharmaceutically acceptable salt thereof, are administered to the subject in the same dosage form or in separate dosage forms. In some embodiments, either one of the compounds can be administered on a daily basis (e.g., as a single dose or as two or more divided doses, e.g., once daily, twice daily, thrice daily) or non-daily basis (e.g., every other day, every two days, every three days, once weekly, twice weekly, once every two weeks, once a month). In some embodiments, the celastrol, or a pharmaceutically acceptable salt thereof, is administered to the subject before the bromodomain inhibitor, or a pharmaceutically acceptable salt thereof. In some embodiments, the celastrol, or a pharmaceutically acceptable salt thereof, is administered to the subject after the bromodomain inhibitor, or a pharmaceutically acceptable salt thereof.

In some embodiments, the bromodomain inhibitor is selected from BRD2 inhibitor, BRD3 inhibitor, BRD4 inhibitor, and BRDT inhibitor. In some embodiments, the bromodomain inhibitor is BRD4 inhibitor. In some embodiments, the bromodomain inhibitor is selected from I-BET 151, JQ1, I-BET 762, OTX-015, TEN-010, CPI-203, CPI-0610, olinone, RVX-208, ABBV-744, LY294002, AZD5153, MT-1, and MS645, or a pharmaceutically acceptable salt thereof. In some embodiments, the bromodomain inhibitor is selected from (+)-JQ1, I-BET762, OTX015, I-BET151, CPI203, PFI-1, MS436, CPI-0610 chemical structures are shown. RVX2135, FT-1101, BAY1238097, INCB054329, TEN-010, GSK2820151, ZEN003694, BAY-299, BMS-986158, ABBV-075, GS-5829, and PLX51107, or a pharmaceutically acceptable salt thereof. In some embodiments, the bromodomain inhibitor is JQ-1 (e.g., “+” or “−” JQ-1), or a pharmaceutically acceptable salt thereof. In some embodiments, the bromodomain inhibitor is JQ-1 having formula:

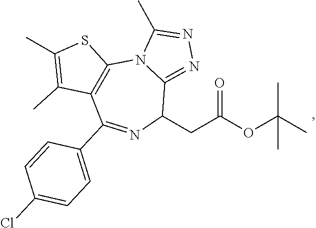 [SMILES: Cc1sc2c(c1C)C(c1ccc(Cl)cc1)=NC(CC(=O)OC(C)(C)C)c1nnc(C)n1-2]

or a pharmaceutically acceptable salt thereof.

In some embodiments, the method includes administering to the subject celastrol, or a pharmaceutically acceptable salt thereof, and JQ-1, or a pharmaceutically acceptable salt thereof.

### Definitions

As used herein, the term “about” means “approximately” (e.g., plus or minus approximately 10% of the indicated value).

At various places in the present specification, substituents of compounds of the invention are disclosed in groups or in ranges. It is specifically intended that the invention include each and every individual subcombination of the members of such groups and ranges. For example, the term “C₁₋₆ alkyl” is specifically intended to individually disclose methyl, ethyl, C₃ alkyl, C₄ alkyl, C₅ alkyl, and C₆ alkyl.

At various places in the present specification various aryl, heteroaryl, cycloalkyl, and heterocycloalkyl rings are described. Unless otherwise specified, these rings can be attached to the rest of the molecule at any ring member as permitted by valency. For example, the term “a pyridine ring” or “pyridinyl” may refer to a pyridin-2-yl, pyridin-3-yl, or pyridin-4-yl ring.

It is further appreciated that certain features of the invention, which are, for clarity, described in the context of separate embodiments, can also be provided in combination in a single embodiment. Conversely, various features of the invention which are, for brevity, described in the context of a single embodiment, can also be provided separately or in any suitable subcombination.

The term “aromatic” refers to a carbocycle or heterocycle having one or more polyunsaturated rings having aromatic character (i.e., having (4n+2) delocalized π (pi) electrons where n is an integer).

The term “n-membered” where n is an integer typically describes the number of ring-forming atoms in a moiety where the number of ring-forming atoms is n. For example, piperidinyl is an example of a 6-membered heterocycloalkyl ring, pyrazolyl is an example of a 5-membered heteroaryl ring, pyridyl is an example of a 6-membered heteroaryl ring, and 1,2,3,4-tetrahydro-naphthalene is an example of a 10-membered cycloalkyl group.

As used herein, the phrase “optionally substituted” means unsubstituted or substituted. The substituents are independently selected, and substitution may be at any chemically accessible position. As used herein, the term “substituted” means that a hydrogen atom is removed and replaced by a substituent. A single divalent substituent, e.g., oxo, can replace two hydrogen atoms. It is to be understood that substitution at a given atom is limited by valency.

Throughout the definitions, the term “Cₙ₋ₘ” indicates a range which includes the endpoints, wherein n and m are integers and indicate the number of carbons. Examples include C₁₋₄, C₁₋₆, and the like.

As used herein, the term “Cₙ₋ₘ alkyl”, employed alone or in combination with other terms, refers to a saturated hydrocarbon group that may be straight-chain or branched, having n to m carbons. Examples of alkyl moieties include, but are not limited to, chemical groups such as methyl, ethyl, n-propyl, isopropyl, n-butyl, tert-butyl, isobutyl, sec-butyl; higher homologs such as 2-methyl-1-butyl, n-pentyl, 3-pentyl, n-hexyl, 1,2,2-trimethylpropyl, and the like. In some embodiments, the alkyl group contains from 1 to 6 carbon atoms, from 1 to 4 carbon atoms, from 1 to 3 carbon atoms, or 1 to 2 carbon atoms.

As used herein, the term “Cₙ₋ₘ haloalkyl”, employed alone or in combination with other terms, refers to an alkyl group having from one halogen atom to 2s+1 halogen atoms which may be the same or different, where “s” is the number of carbon atoms in the alkyl group, wherein the alkyl group has n to m carbon atoms. In some embodiments, the haloalkyl group is fluorinated only. In some embodiments, the alkyl group has 1 to 6, 1 to 4, or 1 to 3 carbon atoms.

As used herein, the term “Cₙ₋ₘ alkylene”, employed alone or in combination with other terms, refers to a divalent alkyl linking group having n to m carbons. Examples of alkylene groups include, but are not limited to, ethan-1,1-diyl, ethan-1,2-diyl, propan-1,1,-diyl, propan-1,3-diyl, propan-1,2-diyl, butan-1,4-diyl, butan-1,3-diyl, butan-1,2-diyl, 2-methyl-propan-1,3-diyl, and the like. In some embodiments, the alkylene moiety contains 2 to 6, 2 to 4, 2 to 3, 1 to 6, 1 to 4, or 1 to 2 carbon atoms.

As used herein, the term “Cₙ₋ₘ alkoxy”, employed alone or in combination with other terms, refers to a group of formula —O-alkyl, wherein the alkyl group has n to m carbons. Example alkoxy groups include, but are not limited to, methoxy, ethoxy, propoxy (e.g., n-propoxy and isopropoxy), butoxy (e.g., n-butoxy and tert-butoxy), and the like. In some embodiments, the alkyl group has 1 to 6, 1 to 4, or 1 to 3 carbon atoms.

As used herein, “Cₙ₋ₘ haloalkoxy” refers to a group of formula —O-haloalkyl having n to m carbon atoms. An example haloalkoxy group is OCF₃. In some embodiments, the haloalkoxy group is fluorinated only. In some embodiments, the alkyl group has 1 to 6, 1 to 4, or 1 to 3 carbon atoms.

As used herein, the term “amino” refers to a group of formula —NH₂.

As used herein, the term “Cₙ₋ₘ alkylamino” refers to a group of formula —NH(alkyl), wherein the alkyl group has n to m carbon atoms. In some embodiments, the alkyl group has 1 to 6, 1 to 4, or 1 to 3 carbon atoms. Examples of alkylamino groups include, but are not limited to, N-methylamino, N-ethylamino, N-propylamino (e.g., N-(n-propyl)amino and N-isopropylamino), N-butylamino (e.g., N-(n-butyl)amino and N-(tert-butyl)amino), and the like.

As used herein, the term “di(Cₙ₋ₘ-alkyl)amino” refers to a group of formula-N(alkyl)₂, wherein the two alkyl groups each has, independently, n to m carbon atoms. In some embodiments, each alkyl group independently has 1 to 6, 1 to 4, or 1 to 3 carbon atoms.

As used herein, “halo” refers to F, Cl, Br, or I. In some embodiments, a halo is F, Cl, or Br.

As used herein, the term “aryl,” employed alone or in combination with other terms, refers to an aromatic hydrocarbon group, which may be monocyclic or polycyclic (e.g., having 2, 3 or 4 fused rings). The term “Cₙ₋ₘaryl” refers to an aryl group having from n to m ring carbon atoms. Aryl groups include, e.g., phenyl, naphthyl, anthracenyl, phenanthrenyl, indanyl, indenyl, and the like. In some embodiments, aryl groups have from 6 to 10 carbon atoms. In some embodiments, the aryl group is phenyl or naphtyl. “Arylene” is a divalent aryl group.

As used herein, “cycloalkyl” refers to non-aromatic cyclic hydrocarbons including cyclized alkyl and/or alkenyl groups. Cycloalkyl groups can include mono- or polycyclic (e.g., having 2, 3 or 4 fused rings) groups and spirocycles. Ring-forming carbon atoms of a cycloalkyl group can be optionally substituted by 1 or 2 independently selected oxo or sulfide groups (e.g., C(O) or C(S)). Also included in the definition of cycloalkyl are moieties that have one or more aromatic rings fused (i.e., having a bond in common with) to the cycloalkyl ring, for example, benzo or thienyl derivatives of cyclopentane, cyclohexane, and the like. A cycloalkyl group containing a fused aromatic ring can be attached through any ring-forming atom including a ring-forming atom of the fused aromatic ring. Cycloalkyl groups can have 3, 4, 5, 6, 7, 8, 9, or 10 ring-forming carbons (C₃₋₁₀). In some embodiments, the cycloalkyl is a C₃₋₁₀ monocyclic or bicyclic cyclocalkyl. In some embodiments, the cycloalkyl is a C₃₋₇ monocyclic cyclocalkyl. Example cycloalkyl groups include cyclopropyl, cyclobutyl, cyclopentyl, cyclohexyl, cycloheptyl, cyclopentenyl, cyclohexenyl, cyclohexadienyl, cycloheptatrienyl, norbornyl, norpinyl, norcarnyl, adamantyl, and the like. In some embodiments, cycloalkyl is cyclopropyl, cyclobutyl, cyclopentyl, or cyclohexyl. “Cycloalkylene” is a divalent cycloalkyl group.

As used herein, “heteroaryl” refers to a monocyclic or polycyclic aromatic heterocycle having at least one heteroatom ring member selected from sulfur, oxygen, and nitrogen. In some embodiments, the heteroaryl ring has 1, 2, 3, or 4 heteroatom ring members independently selected from nitrogen, sulfur and oxygen. In some embodiments, any ring-forming N in a heteroaryl moiety can be an N-oxide. In some embodiments, the heteroaryl is a 5-10 membered monocyclic or bicyclic heteroaryl having 1, 2, 3 or 4 heteroatom ring members independently selected from nitrogen, sulfur and oxygen. In some embodiments, the heteroaryl is a 5-6 monocyclic heteroaryl having 1 or 2 heteroatom ring members independently selected from nitrogen, sulfur and oxygen. In some embodiments, the heteroaryl is a five-membered or six-membereted heteroaryl ring. A five-membered heteroaryl ring is a heteroaryl with a ring having five ring atoms wherein one or more (e.g., 1, 2, or 3) ring atoms are independently selected from N, O, and S. Exemplary five-membered ring heteroaryls are thienyl, furyl, pyrrolyl, imidazolyl, thiazolyl, oxazolyl, pyrazolyl, isothiazolyl, isoxazolyl, 1,2,3-triazolyl, tetrazolyl, 1,2,3-thiadiazolyl, 1,2,3-oxadiazolyl, 1,2,4-triazolyl, 1,2,4-thiadiazolyl, 1,2,4-oxadiazolyl, 1,3,4-triazolyl, 1,3,4-thiadiazolyl, and 1,3,4-oxadiazolyl. A six-membered heteroaryl ring is a heteroaryl with a ring having six ring atoms wherein one or more (e.g., 1, 2, or 3) ring atoms are independently selected from N, O, and S. Exemplary six-membered ring heteroaryls are pyridyl, pyrazinyl, pyrimidinyl, triazinyl and pyridazinyl. “Heteroarylene” is a divalent heteroaryl group.

As used herein, “heterocycloalkyl” refers to non-aromatic monocyclic or polycyclic heterocycles having one or more ring-forming heteroatoms selected from O, N, or S. Included in heterocycloalkyl are monocyclic 4-, 5-, 6-, 7-, 8-, 9- or 10-membered heterocycloalkyl groups. Heterocycloalkyl groups can also include spirocycles. Example heterocycloalkyl groups include pyrrolidin-2-one, 1,3-isoxazolidin-2-one, pyranyl, tetrahydropuran, oxetanyl, azetidinyl, morpholino, thiomorpholino, piperazinyl, tetrahydrofuranyl, tetrahydrothienyl, piperidinyl, pyrrolidinyl, isoxazolidinyl, isothiazolidinyl, pyrazolidinyl, oxazolidinyl, thiazolidinyl, imidazolidinyl, azepanyl, benzazapene, and the like. Ring-forming carbon atoms and heteroatoms of a heterocycloalkyl group can be optionally substituted by 1 or 2 independently selected oxo or sulfido groups (e.g., C(O), S(O), C(S), or S(O)₂, etc.). The heterocycloalkyl group can be attached through a ring-forming carbon atom or a ring-forming heteroatom. In some embodiments, the heterocycloalkyl group contains 0 to 3 double bonds. In some embodiments, the heterocycloalkyl group contains 0 to 2 double bonds. Also included in the definition of heterocycloalkyl are moieties that have one or more aromatic rings fused (i.e., having a bond in common with) to the cycloalkyl ring, for example, benzo or thienyl derivatives of piperidine, morpholine, azepine, etc. A heterocycloalkyl group containing a fused aromatic ring can be attached through any ring-forming atom including a ring-forming atom of the fused aromatic ring. In some embodiments, the heterocycloalkyl is a monocyclic 4-6 membered heterocycloalkyl having 1 or 2 heteroatoms independently selected from nitrogen, oxygen, or sulfur and having one or more oxidized ring members. In some embodiments, the heterocycloalkyl is a monocyclic or bicyclic 4-10 membered heterocycloalkyl having 1, 2, 3, or 4 heteroatoms independently selected from nitrogen, oxygen, or sulfur and having one or more oxidized ring members. “Heterocycloalkylene” is a divalent heterocycloalkyl group.

At certain places, the definitions or embodiments refer to specific rings (e.g., an azetidine ring, a pyridine ring, etc.). Unless otherwise indicated, these rings can be attached to any ring member provided that the valency of the atom is not exceeded. For example, an azetidine ring may be attached at any position of the ring, whereas a pyridin-3-yl ring is attached at the 3-position.

As used herein, the term “oxo” refers to an oxygen atom as a divalent substituent, forming a carbonyl group when attached to a carbon (e.g., C═O), or attached to a heteroatom forming a sulfoxide or sulfone group.

The term “compound” as used herein is meant to include all stereoisomers, geometric isomers, tautomers, and isotopes of the structures depicted. Compounds herein identified by name or structure as one particular tautomeric form are intended to include other tautomeric forms unless otherwise specified.

The compounds described herein can be asymmetric (e.g., having one or more stereocenters). All stereoisomers, such as enantiomers and diastereomers, are intended unless otherwise indicated. Compounds of the present invention that contain asymmetrically substituted carbon atoms can be isolated in optically active or racemic forms. Methods on how to prepare optically active forms from optically inactive starting materials are known in the art, such as by resolution of racemic mixtures or by stereoselective synthesis. Many geometric isomers of olefins, C═N double bonds, N═N double bonds, and the like can also be present in the compounds described herein, and all such stable isomers are contemplated in the present invention. Cis and trans geometric isomers of the compounds of the present invention are described and may be isolated as a mixture of isomers or as separated isomeric forms. In some embodiments, the compound has the (R)-configuration. In some embodiments, the compound has the (S)-configuration.

Compounds provided herein also include tautomeric forms. Tautomeric forms result from the swapping of a single bond with an adjacent double bond together with the concomitant migration of a proton. Tautomeric forms include prototropic tautomers which are isomeric protonation states having the same empirical formula and total charge. Example prototropic tautomers include ketone-enol pairs, amide-imidic acid pairs, lactam-lactim pairs, enamine-imine pairs, and annular forms where a proton can occupy two or more positions of a heterocyclic system, for example, 1H- and 3H-imidazole, 1H-, 2H- and 4H-1,2,4-triazole, 1H- and 2H-isoindole, and 1H- and 2H-pyrazole. Tautomeric forms can be in equilibrium or sterically locked into one form by appropriate substitution.

As used herein, the term “cell” is meant to refer to a cell that is in vitro, ex vivo or in vivo. In some embodiments, an ex vivo cell can be part of a tissue sample excised from an organism such as a mammal. In some embodiments, an in vitro cell can be a cell in a cell culture. In some embodiments, an in vivo cell is a cell living in an organism such as a mammal.

As used herein, the term “contacting” refers to the bringing together of indicated moieties in an in vitro system or an in vivo system. For example, “contacting” the cell with a compound of the invention includes the administration of a compound of the present invention to an individual or patient, such as a human, having the cell, as well as, for example, introducing a compound of the invention into a sample containing a cellular or purified preparation containing the cell.

As used herein, the term “individual”, “patient”, or “subject” used interchangeably, refers to any animal, including mammals, preferably mice, rats, other rodents, rabbits, dogs, cats, swine, cattle, sheep, horses, or primates, and most preferably humans.

As used herein, the phrase “effective amount” or “therapeutically effective amount” refers to the amount of active compound or pharmaceutical agent that elicits the biological or medicinal response in a tissue, system, animal, individual or human that is being sought by a researcher, veterinarian, medical doctor or other clinician.

As used herein the term “treating” or “treatment” refers to 1) inhibiting the disease; for example, inhibiting a disease, condition or disorder in an individual who is experiencing or displaying the pathology or symptomatology of the disease, condition or disorder (i.e., arresting further development of the pathology and/or symptomatology), or 2) ameliorating the disease; for example, ameliorating a disease, condition or disorder in an individual who is experiencing or displaying the pathology or symptomatology of the disease, condition or disorder (i.e., reversing the pathology and/or symptomatology).

As used herein, the term “preventing” or “prevention” of a disease, condition or disorder refers to decreasing the risk of occurrence of the disease, condition or disorder in a subject or group of subjects (e.g., a subject or group of subjects predisposed to or susceptible to the disease, condition or disorder). In some embodiments, preventing a disease, condition or disorder refers to decreasing the possibility of acquiring the disease, condition or disorder and/or its associated symptoms. In some embodiments, preventing a disease, condition or disorder refers to completely or almost completely stopping the disease, condition or disorder from occurring.

### EXAMPLES

### Methods

Reagents and ligands: reagents and ligands were purchased from Chem-Impex International, Millipore-Sigma, TCI America, Beantown Chemical, Combi-Blocks, MedChemExpress, Ontario Chemicals, and BOC Sciences and used as received.

Chemical synthesis: FITC-Ahx-LDEETGEFL-CONH₂ (FITC-KL9) peptide tracer was custom synthesized by Genscript (Piscataway, New Jersey). CDDO-FITC fluorescent tracer was prepared as previously described. Column purifications were performed on a Biotage Isolera 4 Purification System equipped with a 200-400 nm diode array detector. For normal phase flash purifications, Sorbtech Purity Flash Cartridges were used (CFC-52300-012-18 and CFC-52500-025-12). For reverse phase flash purifications, Biotage Sfar Bio C18 Duo 300 A, 20 μm cartridges were used (FSBD-0411-001). Analytical LC/MS was performed on a Waters 2545 HPLC equipped with a 2998 diode array detector, a 2424 evaporative light scattering detector, a 2475 multichannel fluorescence detector, and a Waters 3100 ESI-MS module, using a XTerraMS C18 5 μm, 4.6×50 mm column at a flow rate of 5 mL/min with a linear gradient (95% A: 5% B to 100% B 90 sec and 30 sec hold at 100% B, solvent A=water+0.1% formic acid, solvent B=acetonitrile+0.1% formic acid). LC/MS data analysis was performed using Waters Masslynx V4.1 SCN 846 software. Proton and carbon nuclear magnetic resonance (1H and 13C NMR spectra) were recorded on a Bruker Avance III 400 spectrometer using Topspin 3.2 software and data were analyzed using MestreNova (version 12.0.1-20560, Mestrelab Research). Chemical shifts for protons are reported in parts per million (ppm) and are referenced to residual solvent peaks. Data is reported as follows: chemical shift, multiplicity (s=singlet, br s, =broad singlet, d=doublet, t=triplet, q=quartet, p=pentet, m=multiplet), proton coupling constants (J, Hz), and integration.

Mammalian cell culture: MCF7 cells (ATCC) were propagated in RPMI-1640 medium supplemented with 10% FBS, 1% pen-strep, and 1% L-glutamine at 37° C. and 5% CO₂. MDA-MB-231 cells (ATCC) were propagated in DMEM medium supplemented with 10% FBS, and 1% pen-strep at 37° C. and 5% CO₂.

Preparation of MCF7 cell extracts: a cell pellet from one 15 cm dish (˜25 M cells) of MCF7 cells was allowed to thaw on ice and cells were suspended in 400 μL lysis buffer (25 mM HEPES, 150 mM NaCl, 0.2% (v/v) Triton X-100, 0.02% (v/v) TWEEN-20, pH 7.5 supplemented with 2 mM DTT, 250 U Benzonase (Sigma E1014) and 1 mM AEBSF hydrochloride (Combi-Blocks SS-7834)). Optionally, Roche complete, Mini, EDTA-free protease inhibitor cocktail (Sigma 11836170001) can be used in place of, or in combination with, AEBSF hydrochloride. Cells were homogenized via passage through a 27.5-gauge needle 5 times, and the resulting mixture was incubated with slow, end-over-end mixing at 4° C. for 30 min. The lysate was clarified via centrifugation at 16,100×g for 20 min at 4° C. then 800 μL (1:3 dilution) dilution buffer (25 mM HEPES, 150 mM NaCl, 0.005% (v/v) TWEEN-20, pH 7.5) was added and the lysate was re-clarified at 16,100×g for 20 min at 4° C. Total protein was quantified via detergent-compatible Bradford assay (ThermoFisher 23246) The lysate was used fresh or flash-frozen in liquid nitrogen and stored at −80° C. in single-use aliquots.

TR-FRET measurements: unless otherwise noted, experiments were performed in white, 384-well microtiter plates (Corning 3572) in 30 μL assay volume. TR-FRET measurements were acquired on a Tecan SPARK plate reader with SPARKCONTROL software version V2.1 (Tecan Group Ltd.), with the following settings: 340/50 nm excitation, 490/10 nm (Tb) and 520/10 nm (FITC) emission, 100 us delay, 400 us integration. The 490/10 and 520/10 emission channels were acquired with a 50% mirror and a dichroic 510 mirror, respectively, using independently optimized detector gain settings unless specified otherwise. The TR-FRET ratio was taken as the 520/490 nm intensity ratio on a per-well basis.

Antibody and nanobody labeling: nano-secondary alpaca anti-rabbit IgG (ChromoTek shurbGNHS-1), GST V_{H}H (ChromoTek st-250), anti-6×His IgG (Abcam 18184), and anti-GST IgG (Abcam 19256) were labeled with CoraFluor-1-Pfp as previously described. The following extinction coefficients were used to calculate protein concentration and degree-of-labeling (DOL): ChromoTek shurbGNHS-1 E280=24,075 M⁻¹ cm⁻¹, ChromoTek st-250/280=28,545 M⁻¹ cm⁻¹, IgG/280=210,000 M⁻¹ cm⁻¹, CoraFluor-1-Pfp/340=22,000 M⁻¹ cm⁻¹. Nanobody conjugates were diluted with 50% glycerol and stored at −20° C. IgG conjugates were diluted with 50% glycerol, snap-frozen in liquid nitrogen, and stored at −80° C.

### Example 1—Preparation of Celastrol-Derived BRD4 Degrader

### (2R,4aS,6aS,12bR,14aS,14bR)—N-(1-(4-(4-chlorophenyl)-2,3,9-trimethyl-6H-thieno[3,2-f][1,2,4]triazolo[4,3-a][1,4]diazepin-6-yl)-2-oxo-7,10,13-trioxa-3-azahexadecan-16-yl)-10-hydroxy-2,4a,6a,9,12b,14a-hexamethyl-11-oxo-1,2,3,4,4a,5,6,6a,11,12b,13,14,14a,14b-tetradecahydropicene-2-carboxamide-(CS-JQ1)

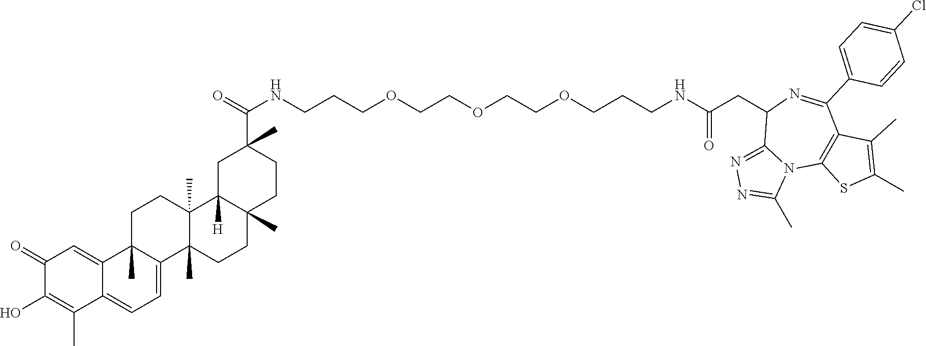

Celastrol (10.0 mg, 22 μmol, 1 eq) was dissolved in DMF (500 μL) then PyBOP (12.1 mg, 23 μmol, 1.05 eq) and 1-(4-(4-chlorophenyl)-2,3,9-trimethyl-6H-thieno[3,2-f][1,2,4]triazolo[4,3-a][1,4]diazepin-6-yl)-2-oxo-7,10,13-trioxa-3-azahexadecan-16-aminium chloride (14.9 mg, 23 μmol, 1.05 eq) were added followed by DIPEA (19 μL, 14 mg, 111 μmol, 5 eq) and the reaction mixture was briefly vortexed then allowed to stand at room temperature for 10 min. The reaction mixture was diluted into EtOAc (50 mL) and the organic layer was washed 2× equal volume 0.2 N HCl, 2×H₂O, 1× saturated brine solution. The organic layer was then dried over Na₂SO₄, filtered, and concentrated. The crude product was purified via reverse phase flash chromatography (A 250 nm, 400 nm; gradient: 5% ACN/H₂O for 3 CV, 5% ACN/H₂O to 100% ACN over 18 CV, 100% ACN for 3 CV). Yield=11.0 mg, 48% as an orange powder. ¹H NMR (400 MHZ, CDCl₃) o 7.40 (d, J=8.2 Hz, 2H), 7.32 (d, J=8.3 Hz, 2H), 7.00 (d, J=6.8 Hz, 2H), 6.88 (t, J=5.0 Hz, 1H), 6.63 (t, J=4.4 Hz, 1H), 6.52 (s, 1H), 6.33 (d, J=7.2 Hz, 1H), 4.64 (t, J=7.0 Hz, 1H), 3.68-3.63 (m, 4H), 3.63-3.57 (m, 6H), 3.53 (t, J=5.9 Hz, 3H), 3.45-3.31 (m, 3H), 3.22 (s, 2H), 2.66 (s, 3H), 2.53-2.43 (m, 1H), 2.40 (s, 3H), 2.20 (s, 3H), 2.17 (s, 3H), 2.11-1.75 (m, 10H), 1.67 (br s, 6H), 1.59-1.48 (m, 5H), 1.42 (s, 3H), 1.12 (s, 3H), 1.09 (s, 3H), 0.63 (s, 3H). ¹³C NMR (101 MHz, CDCl₃) o 178.48, 177.66, 170.61, 170.51, 164.94, 163.95, 155.85, 149.99, 146.14, 136.89, 136.82, 134.24, 132.29, 131.06, 130.92, 130.60, 129.98, 128.84, 127.49, 119.67, 118.12, 117.16, 71.34, 70.65, 70.55, 70.37, 69.96, 54.60, 45.22, 44.55, 43.16, 40.22, 39.53, 39.01, 38.30, 37.97, 36.55, 35.17, 33.92, 33.68, 31.78, 31.23, 31.10, 30.96, 30.12, 29.83, 29.45, 29.18, 28.85, 28.71, 21.88, 18.38, 14.56, 13.25, 12.00, 10.42. MS (ESI^{-/-}) m/z (M+H)⁺ 1035.79, m/z (M−H)⁻1033.47, [calculated C₅₈H₇₅ClN₆O₇S: 1034.51].

### Example 2—Characterization of Celastrol-Derived BRD4 Degrader

Recently, the targeting of other E3 ubiquitin ligase complexes, apart from those canonically used such as CRBN and VHL, using (reversible) covalent ligands that target cysteine side chains has gained increasing attention. Of the predicted >600 E3 ligases, a substantial fraction feature solvent-exposed cysteine residues that can potentially be exploited with corresponding sulfhydryl-reactive biasing elements, including the recently targeted RNF114, RNF4, FEMIB, DCAF16, and DCAF11 complexes.

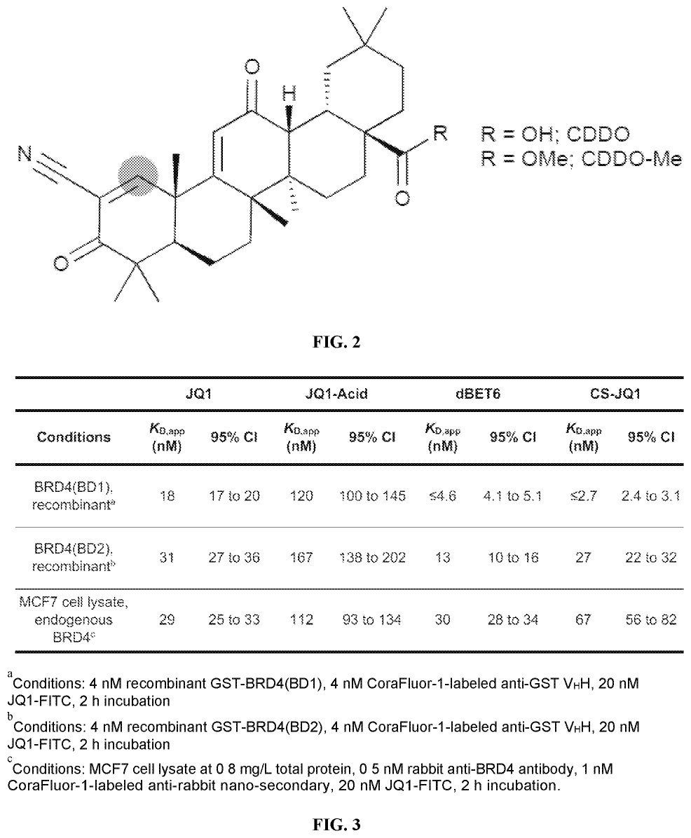

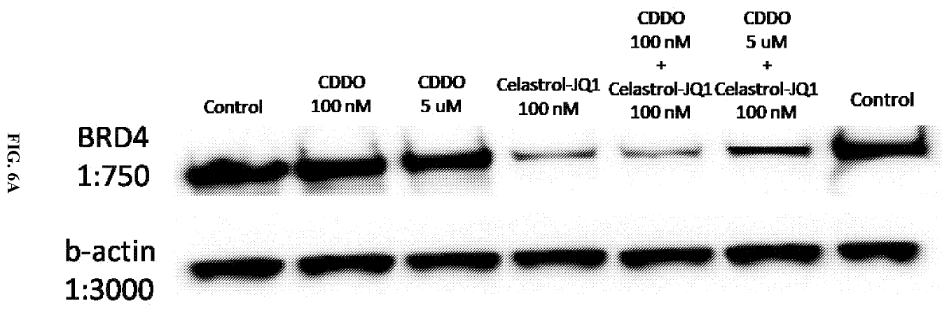

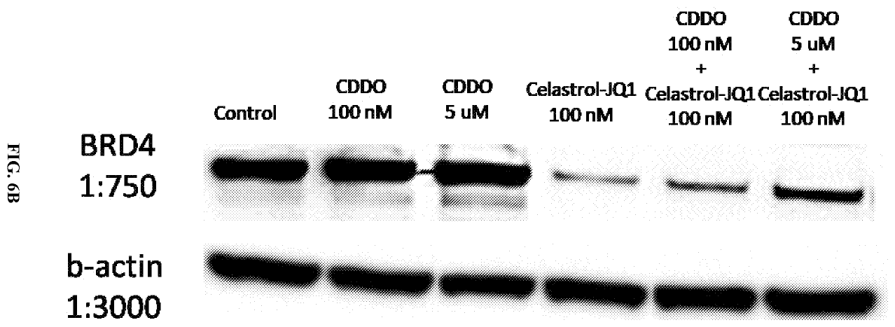

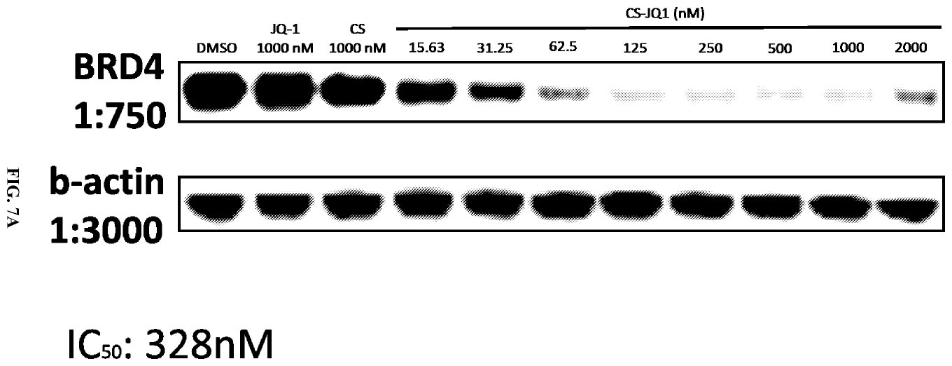

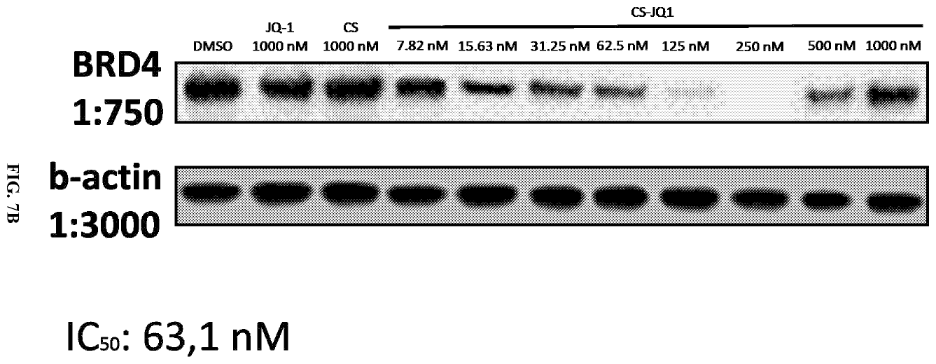

While selective targeting of E3 ligases can potentially provide cell type- or tissue-specific degradation, targeting multiple ligase complexes with a “promiscuous” reversible covalent inhibitor could provide a means for efficient TPD. The triterpenoid celastrol (CS) can form reversible covalent adducts with multiple cysteine nucleophiles and has been shown to bind a host of proteins. CS also targets Keap1 (Kelch-like ECH-associated protein 1), a redox-regulated member of the CRL3 (Cullin-RING E3 ligase) complex that regulates homeostatic abundance of the transcription factor Nrf2 (abbreviation). CS binds Keap1 BTB and Kelch domains with low micromolar affinity (FIG. 5). A previous report has demonstrated the promise of PROTACs (CDDO-JQ1) derived from bardoxolone methyl (CDDO-Me), a synthetic triterpenoid that binds Keap1 with high affinity (FIG. 2). This example shows the capacity of CS to function as a recruiting element for E3 ligase activity in a similar manner.

A PROTAC compound CS-JQ1 (2, FIG. 1A) was prepared as described herein. Data shows that CS-conjugation did not impair binding to BRD4 (FIG. 1B and FIG. 3). Surprisingly and unexpectedly, however, CS-JQ1 lost the ability to bind the Keap1 Kelch domain, while exhibiting slightly improved affinity for the BTB domain (FIG. 1C and FIG. 5). In contrast, functionalization of CDDO results in substantially decreased affinity for BTB. Furthermore, CS-JQ1 was able to induce ternary complex formation between wildtype, full-length Keap1, and isolated BRD4 (BD1) and BRD4 (BD2) domains (FIG. 1D).

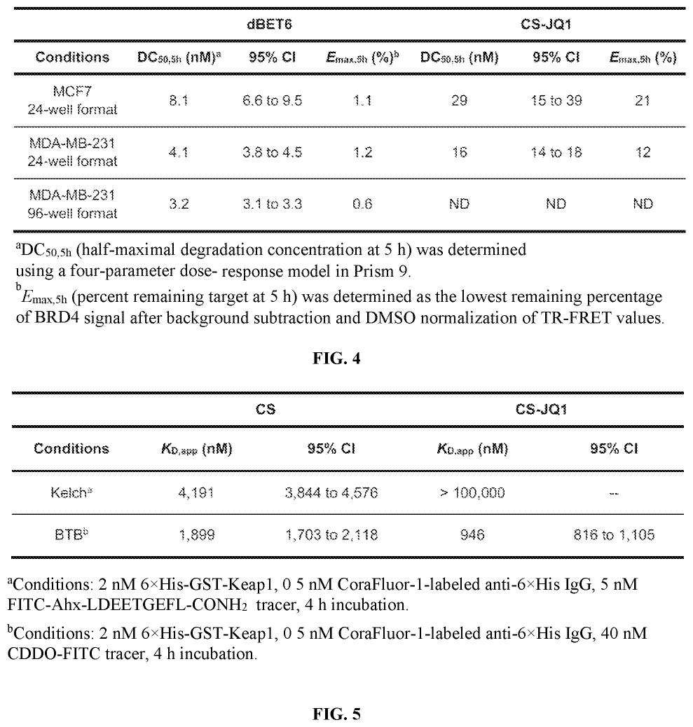

Next, MCF7 and MDA-MB-231 cells were treated for 5 h with varying concentrations of CS-JQ1, followed by BRD4 quantification. CS-JQ1 showed dose-dependent and efficient degradation of BRD4 in both cell lines (DC_{50,5h,MCF7}=29±14 nM; E_{max,5h,MCF7}=21%; DC_{50,5h,MDA-MB-231}=16±2 nM; E_{max,5h,MDA-MB-231}=12%; FIG. 1E and FIG. 4). Western blot analysis on the same samples yielded results that were again in excellent agreement (FIG. 1F-G).

Next, 20S proteasomal, E1 ubiquitin-activating enzyme was evaluated, as well as NEDD8-dependence on CS-JQ1 activity by co-treatment with 1 μM of either BTZ, MLN7243, or MLN4924, respectively (FIG. 1H-I). In both cell lines, CS-JQ1-mediated BRD4 degradation (250 nM) was substantially rescued by all three compounds, indicating proteasomal and ubiquitin system dependence. As expected, co-treatment with JQ1 (10 μM) similarly reduced CS-JQ1 degradation efficiency (FIG. 1H-I). However, when the capacity of CDDO-Me was evaluated (CDDO was shown to bind Keap1 BTB domain with potent activity (K_{D,CDDO-Me}=24 nM)) to attenuate CS-JQ1-induced BRD4 degradation similar to JQ1, it was surprisingly and unexpectedly found that CDDO-Me co-treatment did not increase BRD4 levels.

### DISCUSSION

CS was discovered and validated as a promising new ligand for the redirection of E3 ligase activity. CS-derived degrader exhibits potent activity in reducing cellular BRD4. Additional mechanistic studies showed proteasomal- and ubiquitin system-dependence, providing support that CS-JQ1 acts through the intended mode of action. Interestingly, co-treatment with CDDO-Me did not reduce the efficacy of CS-JQ1. Since the binding affinity of CS and CS-JQ1 for BTB was determined in a ligand displacement assay using a CDDO-based tracer, it was reasonable to assume that CDDO-Me would efficiently displace CS-JQ1. While it cannot be ruled out that CS-JQ1 binds Keap1 via a third, previously unrecognized site, it is more likely that CS-JQ1 is dominantly acting by engaging with one or more other E3 ligase complexes.

### Example 3—CDDO Blockade

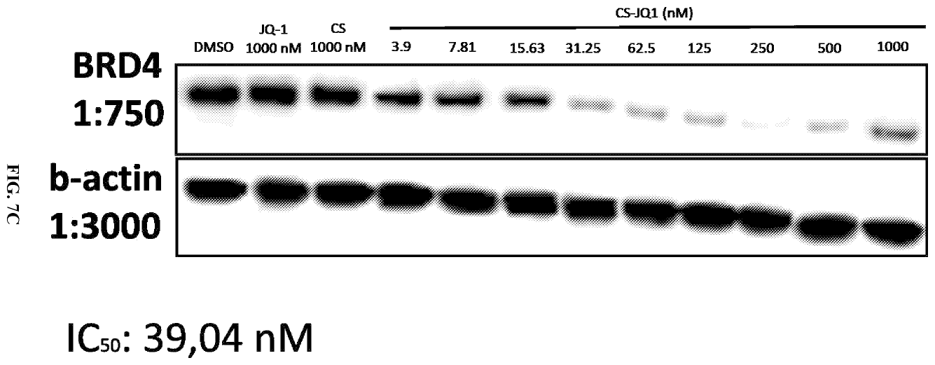

The pre-treatment of cells with CDDO blocks partially the BRD4 degradation induced by CS-JQ1. Referring to FIGS. 6A and 6B, all samples were pre-treated with cicloheximide CHX (3 μg/ml) for 1 hour before an 8 hour treatment with the test compound (JQ1, CS and CS-JQ1).

FIG. 6A shows results of treating MDA (breast cancer) cells (passage 40, 25 μg of protein) with low CDDO (100 nM), high CDDO (5 μm), celastrol-JQ1 (100 nM), low CDDO and celastrol-JQ1, and high CDDO and celastrol-JQ1.

FIG. 6B shows results of treating MES83 (glioma) cells (passage 7, 30 μg of protein) with low CDDO (100 nM), high CDDO (5 μm), celastrol-JQ1 (100 nM), low CDDO and celastrol-JQ1, and high CDDO and celastrol-JQ1.

### Example 4—BRD4 Degradation

Adult glioma cells (MES83 and GBM8), pediatric glioma cells (SJGBM2 and DIPG XIII) and breast cancer cells (MDA-MB-BR and BT474) were pre-treated with cicloheximide (3 μg/ml) for 1 hour before an 8 hour treatment with JQ1, CS and CS-JQ1.

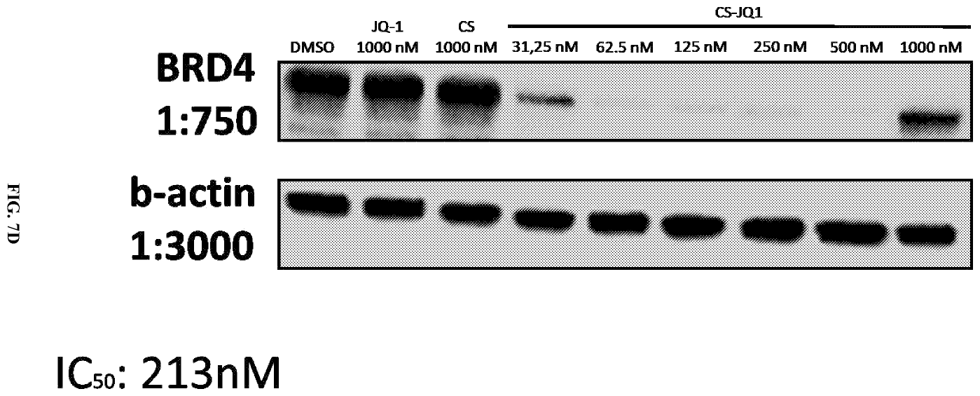

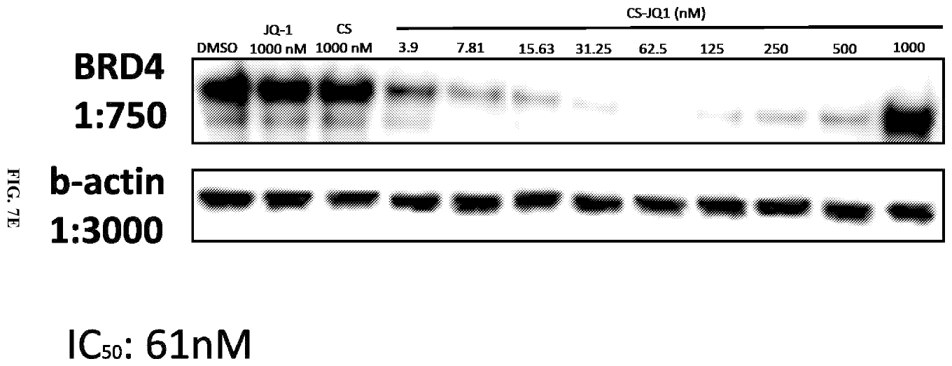

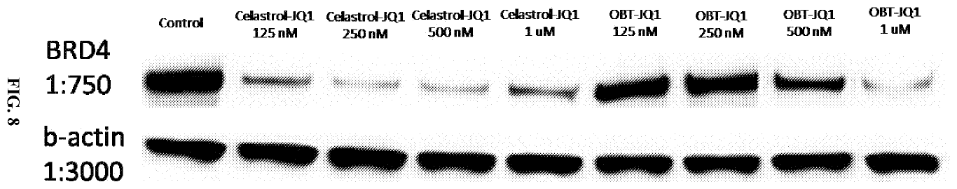

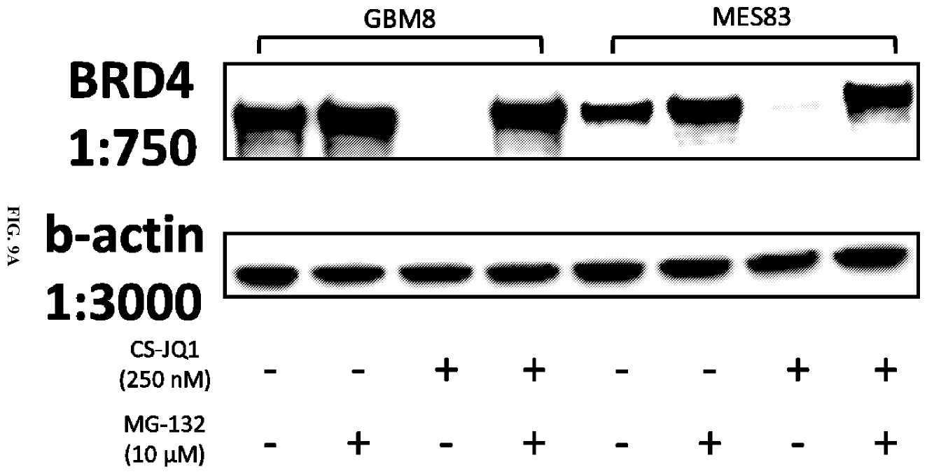

FIG. 7A shows BRD4 degradation after treating MES83 (glioma) cells with JQ1, celastrol, and celastrol-JQ1. FIG. 7B shows BRD4 degradation after treating GBM8 (glioma) cells with JQ1, celastrol, and celastrol-JQ1. FIG. 7C shows BRD4 degradation after treating DIPG XIII (pediatric glioma) cells with JQ1, celastrol, and celastrol-JQ1. FIG. 7D shows BRD4 degradation after treating MDA-MB-BR (metastatic breast cancer) cells with JQ1, celastrol, and celastrol-JQ1. FIG. 7E shows BRD4 degradation after treating BT474 (breast cancer) cells with JQ1, celastrol, and celastrol-JQ1.

### Example 5—BRD4 Degradation

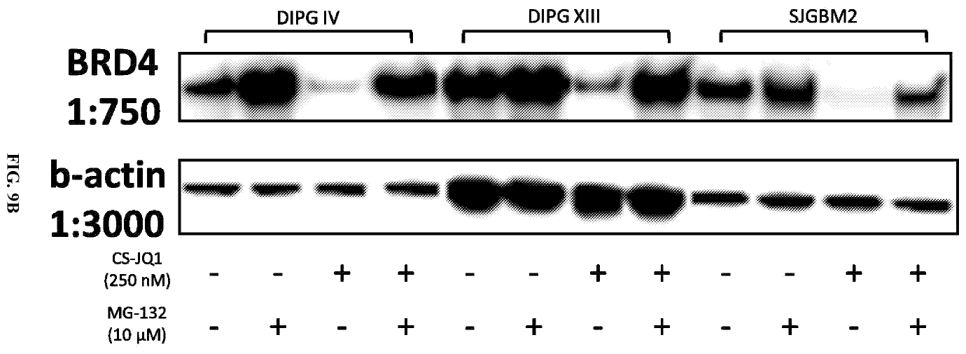

Pediatric glioma cells SJGBM2 at 30 μg of protein per lane were treated with celastrol-JQ1, and obtusaquinone (OBT)-JQ1 (see WO 2021/067799). FIG. 8 shows results of BRD4 degradation at various test compound concentrations. All samples were pre-treated with cicloheximide CHX (3 μg/ml) for 1 h.

### Example 6-comparison between CS-JQ1 and MG-132

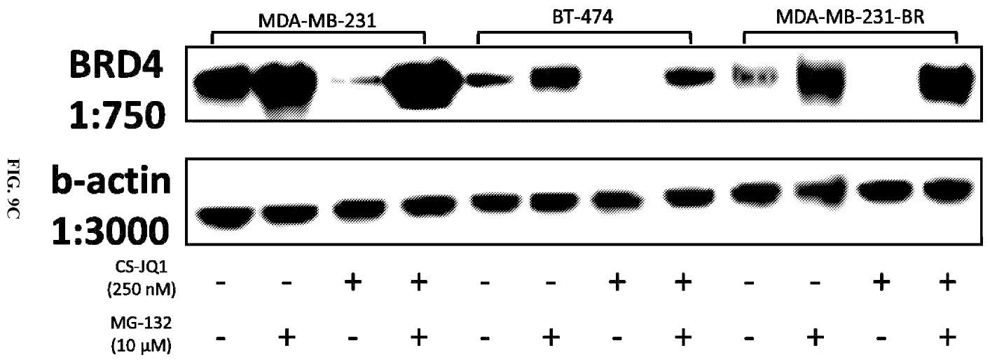

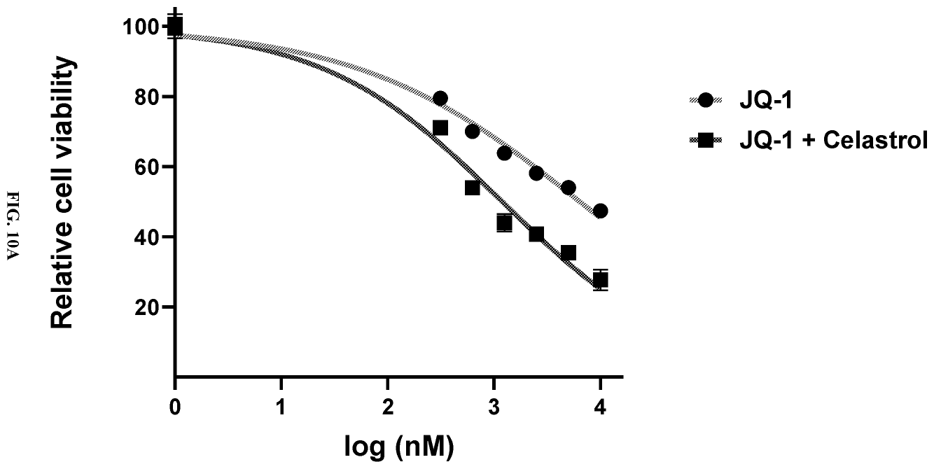

Adult glioma cells (MES83 and GBM8), pediatric glioma cells (SJGBM2, DIPG IV, DIPG XIII) and breast cancer cells (MDA-MB-231, MDA-MB-BR, and BT474) were pre-treated with cicloheximide (3 μg/ml) and MG132 (10 μM) for 1 h before an eight hour treatment with CS-JQ1. FIG. 9A shows BRD4 degradation in different glioma cells by test compounds. FIG. 9B shows BRD4 degradation in different pediatric glioma cells by test compounds. FIG. 9C shows BRD4 degradation in different breast cancer cells by test compounds.

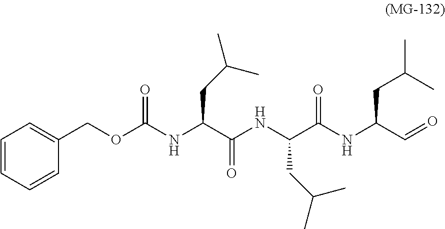 [SMILES: CC(C)C[C@@H](C=O)NC(=O)[C@H](CC(C)C)NC(=O)[C@H](CC(C)C)NC(=O)OCc1ccccc1]

### Example 7—CS-JQ1 Effect on Cell Viability

CS-JQ1 exhibits potent in vitro anti-cancer effect in vitro in breast cancer as well as in pediatric and adult glioma.

TABLE 1
Breast cancer calls

| | IC₅₀ (nM) | | | | |
|---|---|---|---|---|---|
| Cell | JQ-1 | Celastrol | Celastrol-JQ1 | CDDO | CDDO-JQ1 |
| MDA | 6489 | 1197 | 119.4 | 1216 | 747.9 |
| MCF-7 | — | 3317 | 409.4 | >10000 | 6602 |
| MDA-BR | >10000 | 1706 | 212.7 | >10000 | >10000 |
| BT474 | >10000 | 510.6 | 60.94 | 4836 | 1677 |

TABLE 2
Pediatric glioma

| | IC₅₀ (nM) | | | | |
|---|---|---|---|---|---|
| Cell | JQ-1 | Celastrol | Celastrol-JQ1 | CDDO | CDDO-JQ1 |
| SJGBM2 | 1582 | 1188 | 262.9 | 2373 | 635 |
| DIPG XIII | >10000 | 1909 | 39.04 | 1409 | 694.5 |
| | | 1797 | 46.54 | | |
| DIPG VI | >10000 | 1365 | 83.1 | 1334 | 276.6 |
| DIPG IV | 1524 | 2963 | 819.3 | 1356 | 893.9 |
| DIPG 50 | >10000 | 1605 | 298.2 | — | — |
| PHC (mice) | 899.9 | 309.6 | 8.478 | — | — |
| PKC (mice) | 786.1 | 169.8 | 103.3 | — | — |
| XFM (mice) | >10000 | 275.7 | 38.23 | — | — |

TABLE 3
Adult glioma

| | IC₅₀ (nM) | | | | |
|---|---|---|---|---|---|
| Cell | JQ-1 | Celastrol | Celastrol-JQ1 | CDDO | CDDO-JQ1 |
| MES83 | 8117 | 1162 | 328 | 1348 | 1003 |
| GBM8 | — | 714.9 | 63.08 | 578.1 | 123.5 |
| CT2A (mice) | — | 605 | 141.5 | 2118 | 746.6 |
| MES236 | 3679 | 1274 | 190.5 | 1280 | 403.6 |
| MGG29 | >10000 | 1098 | 90.42 | 828.2 | 359.7 |
| PN84 | 604 | 381.2 | 79.7 | 278.2 | 139.4 |
| | | | 124.9 | | |
| U87 | 6949 | 913 | 266.5 | 3700 | 745.6 |
| MGG23 | >10000 | 1814 | 447.3 | 2572 | 1854 |
| MGG6 | — | 1231 | 99.32 | 964.1 | 357.2 |
| M005 (mice) | 750.6 | 873.8 | 23.6 | — | — |

### Example 8—Synergistic Effect of CS and JQ1 Combination

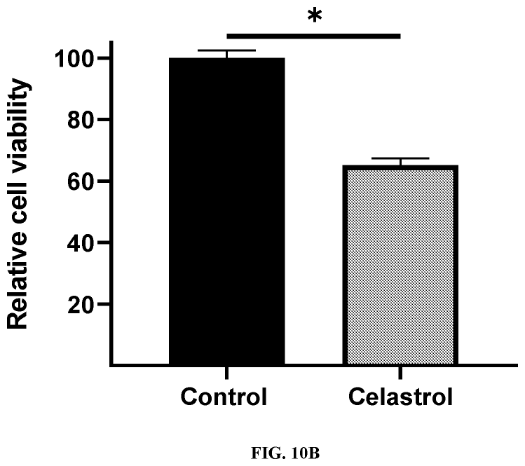

The combination of JQ1 (10, 5, 2.5, 1.25, 0.625, 0.3125 μM) and CS (1 μM) kills cancer cells in an apparent synergistic manner (MES83 and DIPG XIII). FIG. 10A shows relative cell viability for cells treated with either JQ-1 or a combination of JQ-1 and celastrol. FIG. 10B shows relative cell viability for cells treated with either control or celastrol.

| | IC₅₀ (nM) | |
|---|---|---|
| Cell | JQ-1 | JQ-1 + Celastrol |
| DIPG XIII | >10000 | 3272 |
| MES83 | 6379 | 1192 |

### NUMBERED PARAGRAPHS

In some embodiments, the present invention can be described by reference to the following numbered paragraphs:

Paragraph 1. A compound of Formula (I):

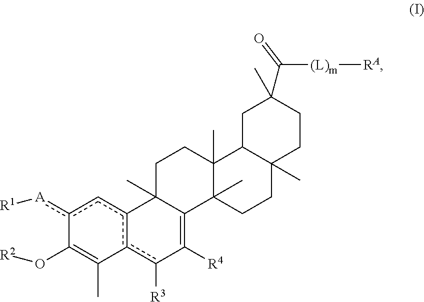

or a pharmaceutically acceptable salt thereof, wherein:
[symbol] indicates a single bond or a double bond;
A is N or O when the bond between A and the carbon to which A is attached is a double bond; or
A is O when the bond between A and the carbon to which A is attached is a single bond;
R¹ is absent when A is O and the bond between A and the carbon to which A is attached is a double bond;
R¹ is H or OH when A is N and the bond between A and the carbon to which A is attached is a double bond; or
R¹ is selected from H, R⁵, COR⁵, COOR⁵, and CONHR⁵, when the bond between A and the carbon to which A is attached is a single bond;
R² is selected from H, R⁵, COR⁵, COOR⁵, and CONHR⁵;
R³ is selected from H, halo, NO₂, CN, OH, SH, OR⁶, SR⁶, C₁₋₆ alkyl, C₁₋₆ haloalkyl, amino, C₁₋₆ alkylamino, di(C₁₋₆ alkyl)amino, CONH₂, CONH(C₁₋₆ alkyl), CON(C₁₋₆ alkyl)₂, COOH, and CO(OC₁₋₆ alkyl);
R⁴ is selected from H, OH, H, halo, NO₂, CN, OH, C₁₋₆ alkyl, C₁₋₆ haloalkyl, C₁₋₆ alkoxy, C₁₋₆ haloalkoxy, amino, C₁₋₆ alkylamino, di(C₁₋₆ alkyl)amino, CONH₂, CONH(C₁₋₆ alkyl), CON(C₁₋₆ alkyl)₂, COOH, and CO(OC₁₋₆ alkyl);
each R⁵ is independently selected from C₁₋₆ alkyl and C₆₋₁₀ aryl, each of which is optionally substituted with 1 or 2 substituents independently selected from NH₂, halo, OH, CN, NO₂, C₁₋₃ alkoxy, OCOC₁₋₆ alkyl, COOH, and CO(OC₁₋₆ alkyl);
each R⁶ is independently selected from R⁵ and COR⁵;
m is an integer from 2 to 20;
each L is independently selected from N(R^{N}), O, C(═O), S, S(═O), S(═O)₂, C₁₋₆ alkylene, C₃₋₇ cycloalkylene, 4-10-membered heterocycloalkylene, 5-10-membered heteroarylene, C₆₋₁₀ arylene, —(OCH₂CH₂)ₓ—, —(CH₂CH₂O)ₓ—, —(OCH(CH₃)CH₂)ₓ—, —(CH₂CH(CH₃)O)ₓ—, each of which is optionally substituted with 1 or 2 substituents independently selected from OH, NH₂, C(O)OH, SO₃H, C₁₋₃ alkylamino, di(C₁₋₃-alkyl)amino, C₁₋₃ haloalkyl, C₁₋₃ alkoxy, and C₁₋₃ haloalkoxy;
each x is independently an integer from 1 to 2,000;
each R^{N} is independently selected from H, C₁₋₆ alkyl, C₂₋₆ alkenyl, and C₂₋₆ alkynyl; and
R^{A} is a targeting ligand capable of selectively binding to a protein.

Paragraph 2. The compound of paragraph 1, wherein the compound has formula:

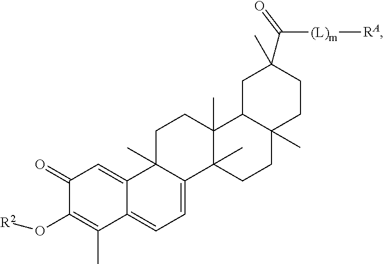

or a pharmaceutically acceptable salt thereof.

Paragraph 3. The compound of paragraph 2, wherein R⁵ is C₁₋₆ alkyl, optionally substituted with NH₂, OH, C₁₋₃ alkoxy, COOH, or CO(OC₁₋₆ alkyl).

Paragraph 4. The compound of paragraph 3, having formula:

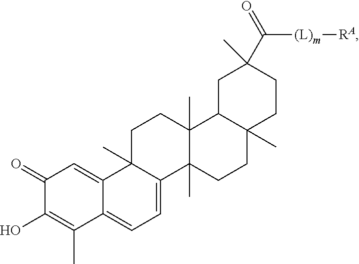

or a pharmaceutically acceptable salt thereof.

Paragraph 5. The compound of paragraph 1, having formula:

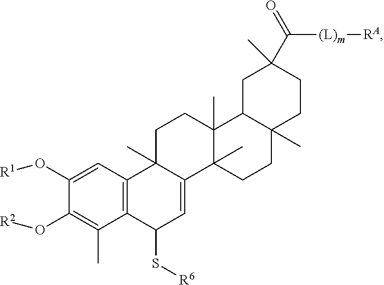

or a pharmaceutically acceptable salt thereof.

Paragraph 6. The compound of paragraph 1, having formula:

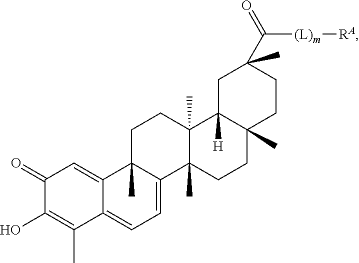

or a pharmaceutically acceptable salt thereof.

Paragraph 7. The compound of any one of paragraphs 1-6, wherein m is 2, 4, 5, 6, or 8.

Paragraph 8. The compound of any one of paragraphs 1-7, wherein each L is independently selected from NH, O, C(═O), C₁₋₆ alkylene, C₃₋₇ cycloalkylene, 4-10-membered heterocycloalkylene, 5-10-membered heteroarylene, C₆₋₁₀ arylene, —(OCH₂CH₂)ₓ—, and —(CH₂CH₂O)ₓ—.

Paragraph 9. The compound of any one of paragraph 1-7, wherein at least one L is C₁₋₆ alkylene.

Paragraph 10. The compound of any one of paragraph 1-7, wherein at least one L is C(═O).

Paragraph 11. The compound of any one of paragraph 1-7, wherein at least one L is 0.

Paragraph 12. The compound of any one of paragraph 1-7, wherein at least one L is —(OCH₂CH₂)ₓ— or —(CH₂CH₂O)ₓ—.

Paragraph 13. The compound of any one of paragraphs 1-12, wherein (L)ₘ comprises at least one 4-10-membered heterocycloalkylene.

Paragraph 14. The compound of any one of paragraphs 1-12, wherein (L)ₘ comprises at least one C₆₋₁₀ arylene.

Paragraph 15. The compound of any one of paragraphs 1-12, wherein (L)ₘ comprises at least one moiety C(═O)O, OC(═O), C(═O)NH, C(═O)NH, NHC(═O)NH, NHC(═S)NH, OC(═O)NH, or NHC(═O)O.

Paragraph 16. The compound of any one of paragraphs 1-15, wherein x is an integer from 1 to 10.

Paragraph 17. The compound of any one of paragraphs 1-6, wherein (L)ₘ moiety comprises any one of the foregoing flexible structural fragments, or any combination thereof:

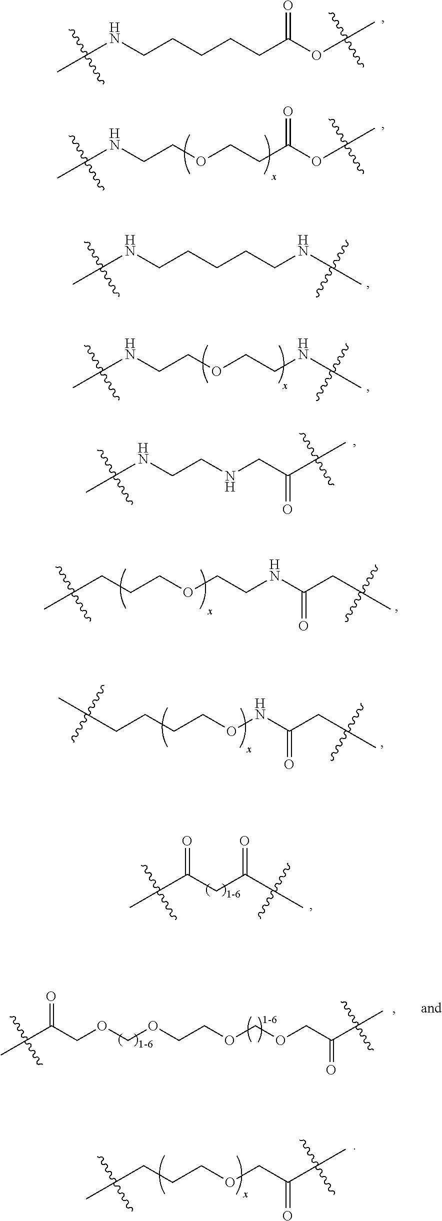

Paragraph 18. The compound of any one of paragraphs 1-6, wherein (L)ₘ moiety comprises any one of the foregoing rigid structural fragments, or any combination thereof:

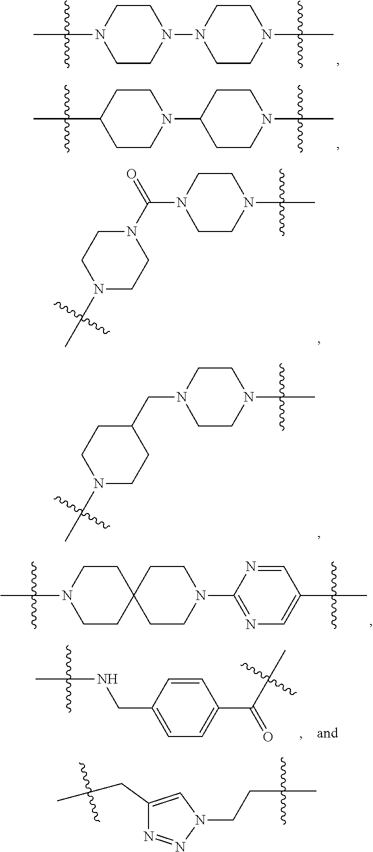

Paragraph 19. The compound of any one of paragraphs 1-18, wherein R^{A} is a ligand capable of binding a pharmacologically relevant protein selected from damaged protein, protein containing a genetic mutation, aggregation-prone protein, and a protein malfunction or excessive function of which substantially contributes to a pathology of a disease.

Paragraph 20. The compound of any one of paragraph 1-18, wherein R^{A} is a targeting ligand capable of selectively binding a protein implicated in the pathology of cancer.

Paragraph 21. The compound of paragraph 20, wherein R^{A} is a targeting ligand capable of selectively binding hormone receptor, androgen receptor (AR), estrogen receptor (ER), estrogen-related receptor alpha (ERRα), KRAS, BRD4 (bromodomain and extraterminal (BET) domain epigenetic reader protein BRD4), BRD2, BRD3, anaplastic lymphoma kinase (ALK), BCL2, BCL6, BCR-ABL, BRD9, BRD7, BTK, CDK4/6, cyclin-dependent kinase 8 (CDK8), cyclin-dependent kinase 9 (CDK9), casein kinase 2 (CK2), c-Met, dihydroorotate dehydrogenase (DHODH), epidermal growth factor receptor (EGFR), human epidermal growth factor receptor 2 (HER2), eukaryotic translation initiation factor 4E (eIF4E), ERK1, ERK2, focal adhesion kinase (FAK), FMS-like tyrosine kinase 3 (FLT3), myeloid cell leukemia 1 (MCL1), murine double minute 2 (MDM2), poly (ADP-ribose) polymerase (PARPs, such as PARP1), transforming acidic coiled-coil containing protein 3 (TACC3), pirin, phosphoinositide 3-kinases (P13Ks), polycomb repressive complex 2 (PRC2), serine-threonine kinase (RIPK2), rpn13, serum/glucocorticoid-inducible protein kinase (SGK), smad3, STAT protein (STAT1, STAT2, STAT3, STAT4, STAT5A, STAT5B, or STAT6), TANK-binding kinase 1 (TBK1), TRIM24, the hepatitis C virus (HCV) NS3 protein, interleukin-1 receptor-associated kinase 4 (IRAK4), P300/CBP-associated factor (PCAF), cellular retinoic acid-binding protein (CRABP-I, -II), anaplastic lymphoma kinase (ALK), mitogen-activated protein kinase 14 (MAPK14, p38-α), mitogen-activated protein kinase 13 (MAPK 13, also known as stress-activated protein kinase 4 (SAPK4), or p38-8), sirtuin, sirtuin2 (SIRT2), P300/CBP associating factor (PCAF), histone deacetylase (e.g., HDAC1, HDAC2, HDAC3, HDAC4, HDAC5, HDAC6,m HDAC7, HDAC8, HDAC9, HDAC10, or HDAC11), cytosolic aminoacyl tRNA synthetase, mitochondrial aminoacyl tRNA synthetase, PD-L1, CD47, cytokine (e.g., IL-2, IL-7, IL-12, IL-15, IL-10, IL-21, or INF-alfa), chemokine (e.g., CCL2, CCL3, or CCL5), or immunosuppressive antigen (e.g., PD-1, CTLA-4, CD20, Lag-3 or Tim-3).

Paragraph 22. The compound of paragraph 20, wherein the targeting ligand R^{A} capable of binding to a target protein implicated in the pathology of cancer is selected from JQ1, VZ185, imatinib, enzalutamide, fulvestrant, tazemetostat, MAK683, UNC1999, adavosertib, AZD1775, carfilzomib, MG-132, apigenin, alectinib, brigatinib, ceritinib, crizotinib, and lorlatinib, TAE684, bosutinib, dasatinib, SNS-032, CX-4945, foretinib, OTX-15, brequinar, lapatinib, gefitinib, afatinib, fulvestrant, defactinib, quizartinib, gilteritinib, MLN-518, sunitinib, ponatinib, MI-1061, olaparib, niraparib, iniparib, veliparib, and bortezomib.

Paragraph 23. The compound of any one of paragraphs 1-15, wherein R^{A} is a targeting ligand capable of selectively binding a protein implicated in the pathology of a neurodegenerative disease or condition.

Paragraph 24. The compound of paragraph 23, wherein R^{A} is a targeting ligand capable of selectively binding alpha-synuclein, transthyretin, tau protein, or amyloid-β peptide.

Paragraph 25. The compound of paragraph 1, wherein the compound of Formula (I) is selected from any one of the following compounds:

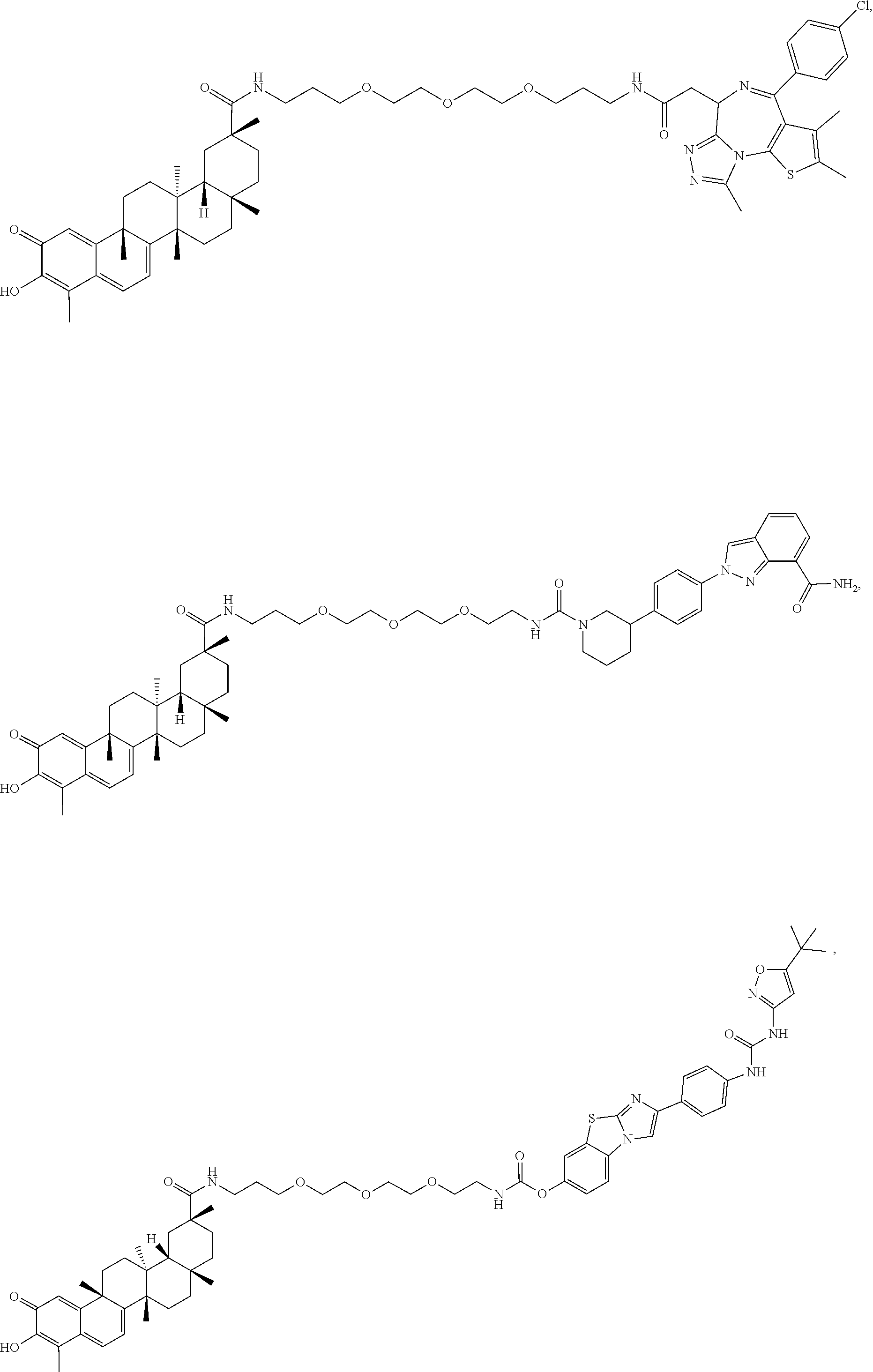

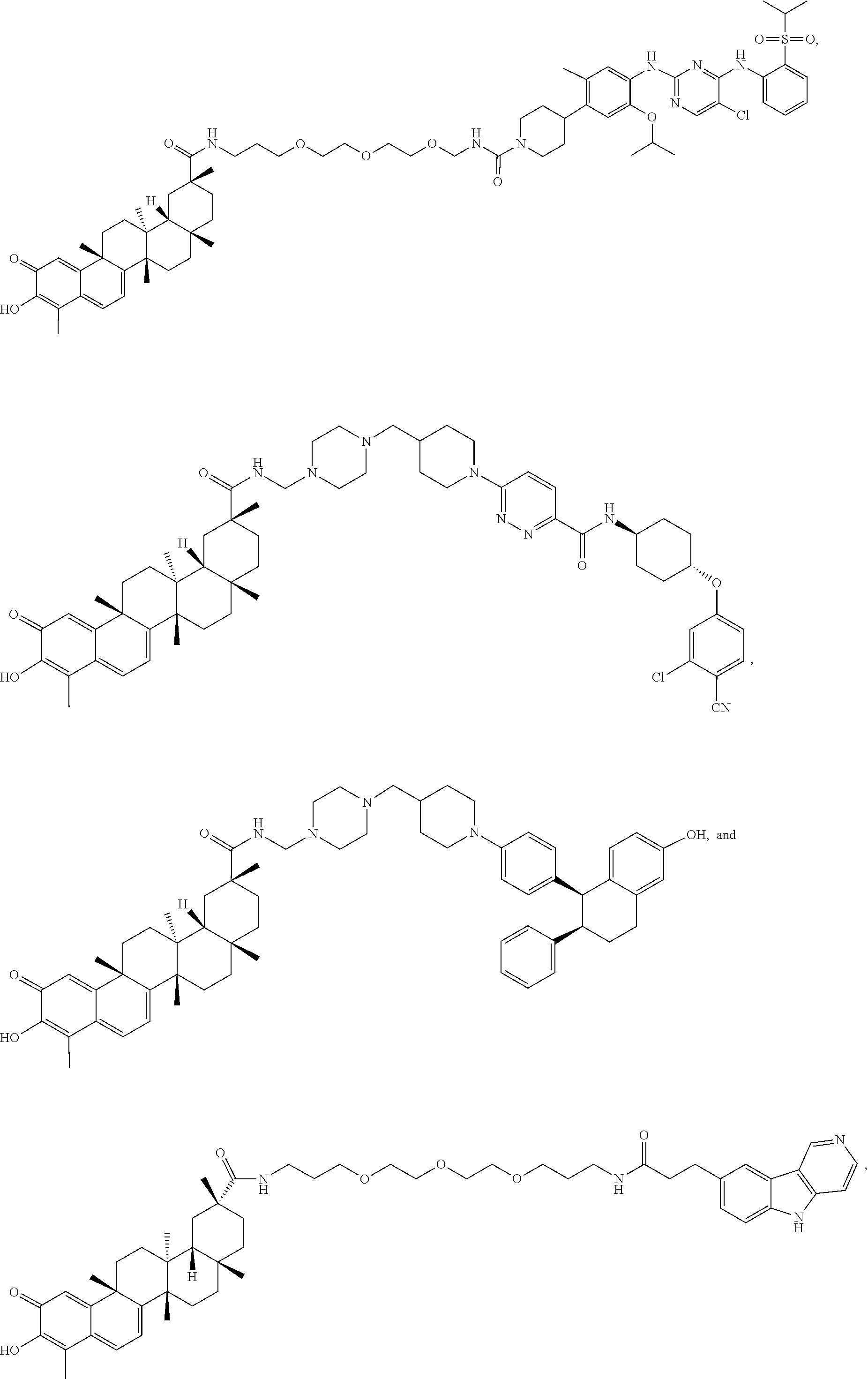

or a pharmaceutically acceptable salt thereof.

Paragraph 26. A pharmaceutical composition comprising a compound of any one of paragraphs 1-25, or a pharmaceutically acceptable salt thereof, and a pharmaceutically acceptable carrier.

Paragraph 27. A method of treating a disease or condition, the method comprising administering to a subject in need thereof a therapeutically effective amount of a compound of any one of paragraphs 1-25, or a pharmaceutically acceptable salt thereof, wherein the protein is implicated in the pathology of the disease or condition.

Paragraph 28. The method of paragraph 27, wherein the disease or condition is cancer.

Paragraph 29. The method of paragraph 28, wherein the cancer is selected from bladder cancer, brain cancer, breast cancer, colorectal cancer, cervical cancer, gastrointestinal cancer, genitourinary cancer, head and neck cancer, lung cancer, ovarian cancer, pancreatic cancer, prostate cancer, renal cancer, skin cancer, and testicular cancer. In some embodiments, the cancer is selected from sarcoma, angiosarcoma, fibrosarcoma, rhabdomyosarcoma, liposarcoma, myxoma, rhabdomyoma, fibroma, lipoma, teratoma, lung cancer, bronchogenic carcinoma squamous cell, undifferentiated small cell, undifferentiated large cell, adenocarcinoma, alveolar bronchiolar carcinoma, bronchial adenoma, sarcoma, lymphoma, chondromatous hamartoma, mesothelioma, gastrointestinal cancer, cancer of the esophagus, squamous cell carcinoma, adenocarcinoma, leiomyosarcoma, lymphoma, cancer of the stomach, carcinoma, lymphoma, leiomyosarcoma, cancer of the pancreas, ductal adenocarcinoma, insulinoma, glucagonoma, gastrinoma, carcinoid tumor, vipoma, cancer of the small bowel, adenocarcinoma, lymphoma, carcinoid tumors, Kaposi's sarcoma, leiomyoma, hemangioma, lipoma, neurofibroma, fibroma, cancer of the large bowel or colon, tubular adenoma, villous adenoma, hamartoma, leiomyoma, genitourinary tract cancer, cancer of the kidney adenocarcinoma, Wilm's tumor (nephroblastoma), cancer of the bladder, cancer of the urethra, squamous cell carcinoma, transitional cell carcinoma, cancer of the prostate, cancer of the testis, seminoma, teratoma, embryonal carcinoma, teratocarcinoma, choriocarcinoma, sarcoma, interstitial cell carcinoma, fibroma, fibroadenoma, adenomatoid tumors, lipoma, liver cancer, hepatoma hepatocellular carcinoma, cholangiocarcinoma, hepatoblastoma, angiosarcoma, hepatocellular adenoma, hemangioma, bone cancer, osteogenic sarcoma (osteosarcoma), fibrosarcoma, malignant fibrous histiocytoma, chondrosarcoma, Ewing's sarcoma, malignant lymphoma (reticulum cell sarcoma), malignant giant cell tumor, chordoma, osteochrondroma (osteocartilaginous exostoses), benign chondroma, chondroblastoma, chondromyxofibroma, osteoid osteoma giant cell tumor, nervous system cancer, cancer of the skull, osteoma, hemangioma, granuloma, xanthoma, osteitis deformans, cancer of the meninges meningioma, meningiosarcoma, gliomatosis, cancer of the brain, astrocytoma, medulloblastoma, glioma, pediatric glioma, ependymoma, germinoma (pinealoma), glioblastoma multiforme, oligodendroglioma, schwannoma, retinoblastoma, congenital tumors, cancer of the spinal cord, neurofibroma, meningioma, sarcoma, gynecological cancer, cancer of the uterus, endometrial carcinoma, cancer of the cervix, cervical carcinoma, pre tumor cervical dysplasia, cancer of the ovaries, ovarian carcinoma, serous cystadenocarcinoma, mucinous cystadenocarcinoma, unclassified carcinoma, granulosa-theca cell tumor, Sertoli Leydig cell tumor, dysgerminoma, malignant teratoma, cancer of the vulva, squamous cell carcinoma, intraepithelial carcinoma, adenocarcinoma, fibrosarcoma, melanoma, cancer of the vagina, clear cell carcinoma, squamous cell carcinoma, botryoid sarcoma, embryonal rhabdomyosarcoma, cancer of the fallopian tubes, hematologic cancer, cancer of the blood, lymphoma, leukemia, acute myeloid leukemia (AML), chronic myeloid leukemia (CML), acute lymphoblastic leukemia (ALL), chronic lymphoblastic leukemia, chronic lymphocytic leukemia, myeloproliferative diseases, multiple myeloma, myelodysplastic syndrome, Hodgkin's lymphoma, non-Hodgkin's lymphoma (malignant lymphoma), Waldenstrom's macroglobulinemia, skin cancer, malignant melanoma, basal cell carcinoma, squamous cell carcinoma, Kaposi's sarcoma, moles dysplastic nevi, lipoma, angioma, dermatofibroma, keloids, adrenal gland cancer, and neuroblastoma.

Paragraph 30. The method of paragraph 27, wherein the disease or condition is a neurodegenerative disease or condition.

Paragraph 31 The method of paragraph 30 wherein the neurodegenerative disease or condition is selected from Huntington's disease (HD), Alzheimer's disease (AD), Parkinson's disease (PD), multiple sclerosis (MS), frontotemporal dementia (FTD), amyotrophic lateral sclerosis (ALS), Lewy body disease, dementia, motor neuron disease (MND), Prion disease, cerebral amyloid angiopathy, vascular cognitive impairment (VCI), hippocampal sclerosis, Binswanger's disease, Creutzfeldt-Jakob disease, cerebral ischemia, cerebrovascular ischemia, brain ischemia, cerebral palsy, chemotherapy-induced brain damage; cisplatin-induced neurotoxicity, diabetic neuropathy; Down's syndrome, epilepsy and post-traumatic epilepsy; Friedreich's ataxia; Hallervorden-Spatz disease; macular degeneration; methanol-induced neurotoxicity; meningitis (aseptic and tuberculous); Pick's disease; progressive supra-nuclear palsy; radiotherapy-induced brain damage; senile dementia; schizophrenia; traumatic brain injury (TBI); traumatic spinal injury; viral meningitis; encephalitis, and viral encephalitis.

Paragraph 32. The method of any one of paragraphs 27-31, comprising administering to the subject a second therapeutic useful in treating the disease or condition.

Paragraph 33. A method of treating cancer, the method comprising administering a subject in need thereof:
(i) a therapeutically effective amount of celastrol having formula:

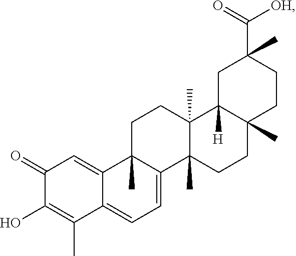

or a pharmaceutically acceptable salt thereof, and
(ii) a therapeutically effective amount of a bromodomain inhibitor, or a pharmaceutically acceptable salt thereof.

Paragraph 34. The method of paragraph 33, wherein the cancer is selected from any of the cancers as recited in paragraph 29.

Paragraph 34. The method of paragraph 33, wherein the cancer is selected from breast cancer, glioma, and pediatric glioma.

Paragraph 35. The method of paragraph 33 or 34, wherein the bromodomain inhibitor is selected from BRD2 inhibitor, BRD3 inhibitor, BRD4 inhibitor, and BRDT inhibitor.

Paragraph 36. The method of paragraph 33 or 34, wherein the bromodomain inhibitor is selected from I-BET 151, JQ1, I-BET 762, OTX-015, TEN-010, CPI-203, CPI-0610, olinone, RVX-208, ABBV-744, LY294002, AZD5153, MT-1, and MS645, or a pharmaceutically acceptable salt thereof.

Paragraph 37. The method of paragraph 33 or 34, wherein the bromodomain inhibitor is JQ-1 having formula:

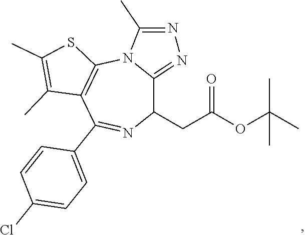 [SMILES: Cc1sc2c(c1C)C(c1ccc(Cl)cc1)=NC(CC(=O)OC(C)(C)C)c1nnc(C)n1-2]

or a pharmaceutically acceptable salt thereof.

Paragraph 38. The method of any one of paragraphs 33-37, wherein the celastrol, or a pharmaceutically acceptable salt thereof, and the bromodomain inhibitor, or a pharmaceutically acceptable salt thereof, are administered to the subject simultaneously.

Paragraph 39. The method of paragraph 38, wherein the celastrol, or a pharmaceutically acceptable salt thereof, and the bromodomain inhibitor, or a pharmaceutically acceptable salt thereof, are administered to the subject in the same dosage form or in separate dosage forms.

Paragraph 40. The method of any one of paragraphs 33-37, wherein the celastrol, or a pharmaceutically acceptable salt thereof, and the bromodomain inhibitor, or a pharmaceutically acceptable salt thereof, are administered to the subject consecutively.

### OTHER EMBODIMENTS

It is to be understood that while the present application has been described in conjunction with the detailed description thereof, the foregoing description is intended to illustrate and not limit the scope of the present application, which is defined by the scope of the appended claims. Other aspects, advantages, and modifications are within the scope of the following claims.

What is claimed is:

1. A compound of Formula (I): 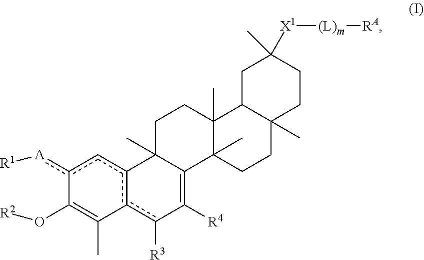
or a pharmaceutically acceptable salt thereof, wherein:
X¹ is NR^{N} or C(═O);
R^{N} is selected from H, C₁₋₃ alkyl, and C₁₋₃ haloalkyl; [symbol]
indicates a single bond or a double bond;
A is N or O when the bond between A and the carbon to which A is attached is a double bond; or
A is O when the bond between A and the carbon to which A is attached is a single bond;
R¹ is absent when A is O and the bond between A and the carbon to which A is attached is a double bond;
R¹ is H or OH when A is N and the bond between A and the carbon to which A is attached is a double bond; or
R¹ is selected from H, R⁵, COR⁵, COOR⁵, and CONHR⁵, when the bond between A and the carbon to which A is attached is a single bond;
R² is selected from H, R⁵, COR⁵, COOR⁵, and CONHR⁵;
R³ is selected from H, halo, NO₂, CN, OH, SH, OR⁶, SR⁶, C₁₋₆ alkyl, C₁₋₆ haloalkyl, amino, C₁₋₆ alkylamino, di(C₁₋₆ alkyl)amino, CONH₂, CONH(C₁₋₆ alkyl), CON(C₁₋₆ alkyl)₂, COOH, and CO(OC₁₋₆ alkyl);
R⁴ is selected from H, OH, H, halo, NO₂, CN, OH, C₁₋₆ alkyl, C₁₋₆ haloalkyl, C₁₋₆ alkoxy, C₁₋₆ haloalkoxy, amino, C₁₋₆ alkylamino, di(C₁₋₆ alkyl)amino, CONH₂, CONH(C₁₋₆ alkyl), CON(C₁₋₆ alkyl)₂, COOH, and CO(OC₁₋₆ alkyl);
each R⁵ is independently selected from C₁₋₆ alkyl and C₆₋₁₀ aryl, each of which is optionally substituted with 1 or 2 substituents independently selected from NH₂, halo, OH, CN, NO₂, C₁₋₃ alkoxy, OCOC₁₋₆ alkyl, COOH, and CO(OC₁₋₆ alkyl);
each R⁶ is independently selected from R⁵ and COR⁵;
m is an integer from 2 to 20;
each L is independently selected from N(R^{N}), O, C(═O), S, S(═O), S(═O)₂, C₁₋₆ alkylene, C₃₋₇ cycloalkylene, 4-10-membered heterocycloalkylene, 5-10-membered heteroarylene, C₆₋₁₀ arylene, —(OCH₂CH₂)ₓ—, —(CH₂CH₂O)ₓ—, —(OCH(CH₃)CH₂)ₓ—, —(CH₂CH(CH₃)O)ₓ—, each of which is optionally substituted with 1 or 2 substituents independently selected from OH, NH₂, C(O)OH, SO₃H, C₁₋₃ alkylamino, di(C₁₋₃-alkyl)amino, C₁₋₃ haloalkyl, C₁₋₃ alkoxy, and C₁₋₃ haloalkoxy;
each x is independently an integer from 1 to 2,000;
each R^{N} is independently selected from H, C₁₋₆ alkyl, C₂₋₆ alkenyl, and C₂₋₆ alkynyl; and
R^{A} is a targeting ligand capable of selectively binding to a protein.

2. The compound of claim 1, wherein the compound is selected from any of the following formulae: 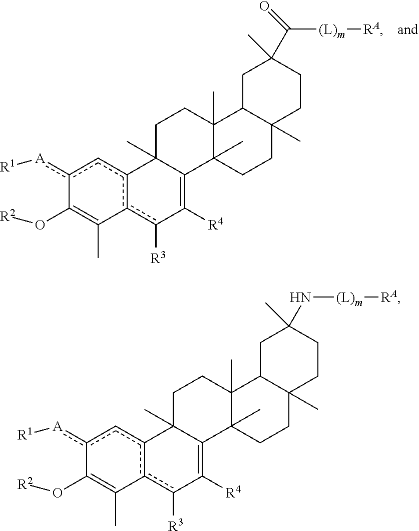
or a pharmaceutically acceptable salt thereof.

3. The compound of claim 1, wherein the compound is selected from any of the following formulae: 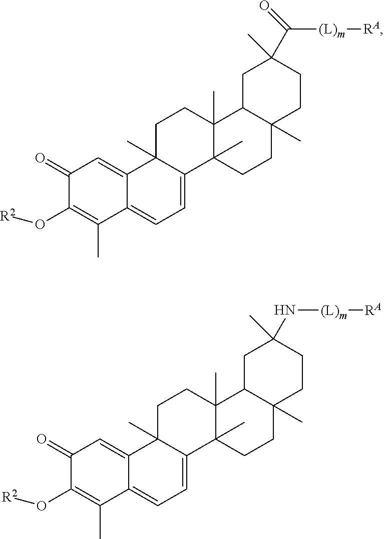
or a pharmaceutically acceptable salt thereof.

4. The compound of any one of claims 1-3, wherein R⁵ is C₁₋₄ alkyl, optionally substituted with NH₂, OH, C₁₋₃ alkoxy, COOH, or CO(OC₁₋₆ alkyl).

5. The compound of claim 1, wherein the compound is selected from any of the following formulae: 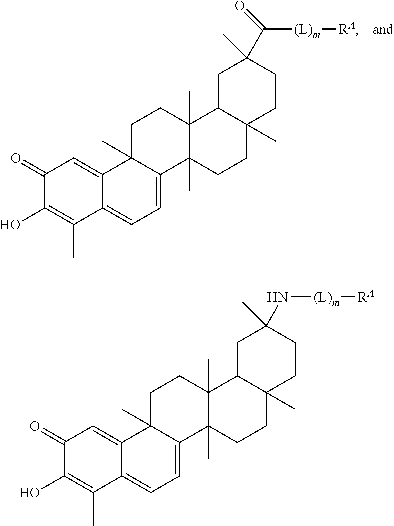
or a pharmaceutically acceptable salt thereof.

6. The compound of claim 1, wherein the compound is selected from any of the following formulae: 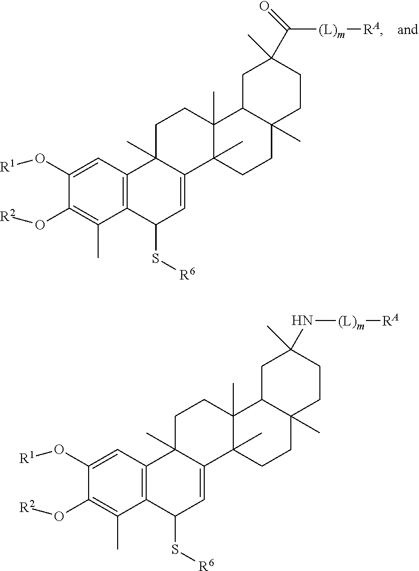
or a pharmaceutically acceptable salt thereof.

7. The compound of claim 1, wherein the compound is selected from any of the following formulae: 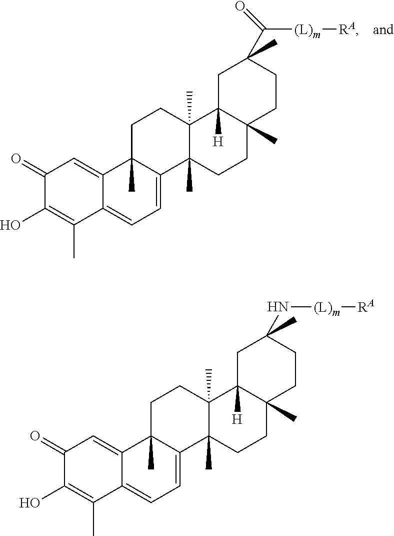
or a pharmaceutically acceptable salt thereof.

8. The compound of any one of claims 1-7, wherein:
m is 2, 4, 5, 6, or 8;
each L is independently selected from NH, O, C(═O), C₁₋₆ alkylene, C₃₋₇ cycloalkylene, 4-10-membered heterocycloalkylene, 5-10-membered heteroarylene, C₆₋₁₀ arylene, —(OCH₂CH₂)ₓ—, and —(CH₂CH₂O)ₓ—; and
is an integer from 1 to 10.

9. The compound of any one of claim 1-8, wherein
at least one L is C₁₋₆ alkylene;
at least one L is C(═O)O; or
at least one L is —(OCH₂CH₂)ₓ— or —(CH₂CH₂O)ₓ—; and
is an integer from 1 to 10.

10. The compound of any one of claims 1-9, wherein:
(L)ₘ comprises at least one 4-10-membered heterocycloalkylene; or
(L)ₘ comprises at least one C₆₋₁₀ arylene; (L)ₘ comprises at least one moiety C(═O)O, OC(═O), C(═O)NH, C(═O)NH, NHC(═O)NH, NHC(═S)NH, OC(═O)NH, or NHC(═O)O; and
is an integer from 1 to 10.

11. The compound of any one of claims 1-7, wherein (L)ₘ moiety comprises any one of the foregoing flexible structural fragments, or any combination thereof:

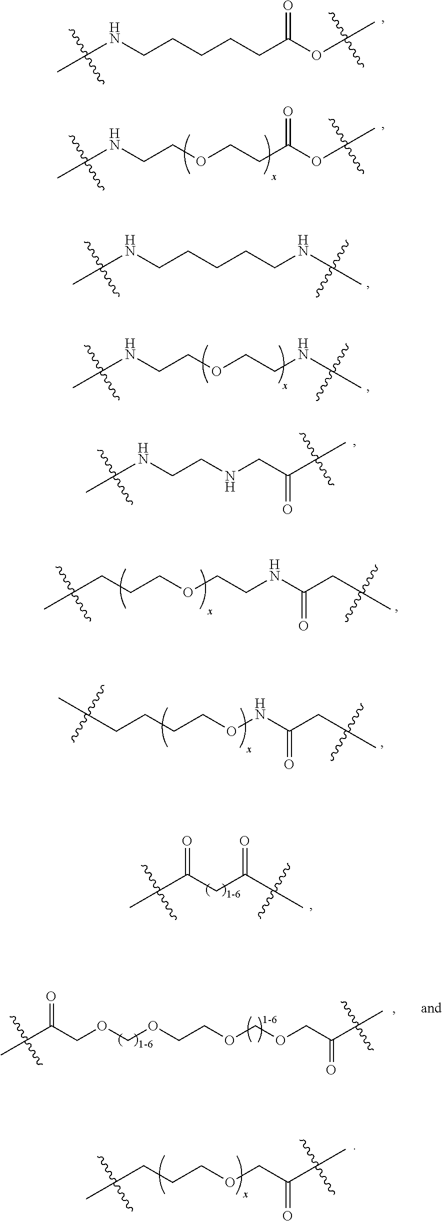

12. The compound of any one of claims 1-7, wherein (L)ₘ moiety comprises any one of the foregoing rigid structural fragments, or any combination thereof:

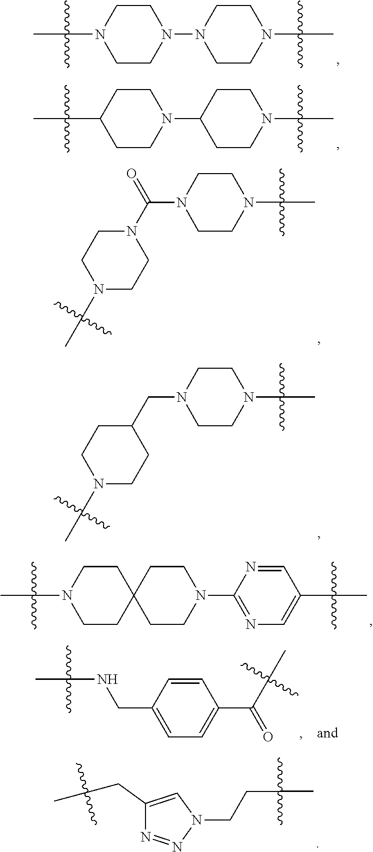

13. The compound of any one of claims 1-12, wherein R^{A} is a ligand capable of binding a pharmacologically relevant protein selected from damaged protein, protein containing a genetic mutation, aggregation-prone protein, and a protein malfunction or excessive function of which substantially contributes to a pathology of a disease.

14. The compound of any one of claim 1-13, wherein R^{A} is a targeting ligand capable of selectively binding a protein implicated in the pathology of cancer.

15. The compound of claim 14, wherein R^{A} is a targeting ligand capable of selectively binding hormone receptor, androgen receptor (AR), estrogen receptor (ER), estrogen-related receptor alpha (ERRα), KRAS, BRD4 (bromodomain and extraterminal (BET) domain epigenetic reader protein BRD4), BRD2, BRD3, anaplastic lymphoma kinase (ALK), BCL2, BCL6, BCR-ABL, BRD9, BRD7, BTK, CDK4/6, cyclin-dependent kinase 8 (CDK8), cyclin-dependent kinase 9 (CDK9), casein kinase 2 (CK2), c-Met, dihydroorotate dehydrogenase (DHODH), epidermal growth factor receptor (EGFR), human epidermal growth factor receptor 2 (HER2), eukaryotic translation initiation factor 4E (eIF4E), ERK1, ERK2, focal adhesion kinase (FAK), FMS-like tyrosine kinase 3 (FLT3), myeloid cell leukemia 1 (MCL1), murine double minute 2 (MDM2), poly (ADP-ribose) polymerase (PARPs, such as PARP1), transforming acidic coiled-coil containing protein 3 (TACC3), pirin, phosphoinositide 3-kinases (P13Ks), polycomb repressive complex 2 (PRC2), serine-threonine kinase (RIPK2), rpn13, serum/glucocorticoid-inducible protein kinase (SGK), smad3, STAT protein (STAT1, STAT2, STAT3, STAT4, STAT5A, STAT5B, or STAT6), TANK-binding kinase 1 (TBK1), TRIM24, the hepatitis C virus (HCV) NS3 protein, interleukin-1 receptor-associated kinase 4 (IRAK4), P300/CBP-associated factor (PCAF), cellular retinoic acid-binding protein (CRABP-I, -II), anaplastic lymphoma kinase (ALK), mitogen-activated protein kinase 14 (MAPK14, p38-α), mitogen-activated protein kinase 13 (MAPK 13, also known as stress-activated protein kinase 4 (SAPK4), or p38-δ), sirtuin, sirtuin2 (SIRT2), P300/CBP associating factor (PCAF), histone deacetylase (e.g., HDAC1, HDAC2, HDAC3, HDAC4, HDAC5, HDAC6,m HDAC7, HDAC8, HDAC9, HDAC10, or HDAC11), cytosolic aminoacyl tRNA synthetase, mitochondrial aminoacyl tRNA synthetase, PD-L1, CD47, cytokine (e.g., IL-2, IL-7, IL-12, IL-15, IL-10, IL-21, or INF-alfa), chemokine (e.g., CCL2, CCL3, or CCL5), or immunosuppressive antigen (e.g., PD-1, CTLA-4, CD20, Lag-3 or Tim-3).

16. The compound of claim 15, wherein the targeting ligand R^{A} capable of binding to a target protein implicated in the pathology of cancer is selected from JQ1, VZ185, imatinib, enzalutamide, fulvestrant, tazemetostat, MAK683, UNC1999, adavosertib, AZD1775, carfilzomib, MG-132, apigenin, alectinib, brigatinib, ceritinib, crizotinib, and lorlatinib, TAE684, bosutinib, dasatinib, SNS-032, CX-4945, foretinib, OTX-15, brequinar, lapatinib, gefitinib, afatinib, fulvestrant, defactinib, quizartinib, gilteritinib, MLN-518, sunitinib, ponatinib, MI-1061, olaparib, niraparib, iniparib, veliparib, and bortezomib.

17. The compound of any one of claims 1-13, wherein R^{A} is a targeting ligand capable of selectively binding a protein implicated in the pathology of a neurodegenerative disease or condition.

18. The compound of claim 17, wherein R^{A} is a targeting ligand capable of selectively binding alpha-synuclein, transthyretin, tau protein, or amyloid-β peptide.

19. The compound of claim 1, wherein the compound of Formula (I) is selected from any one of the following compounds: 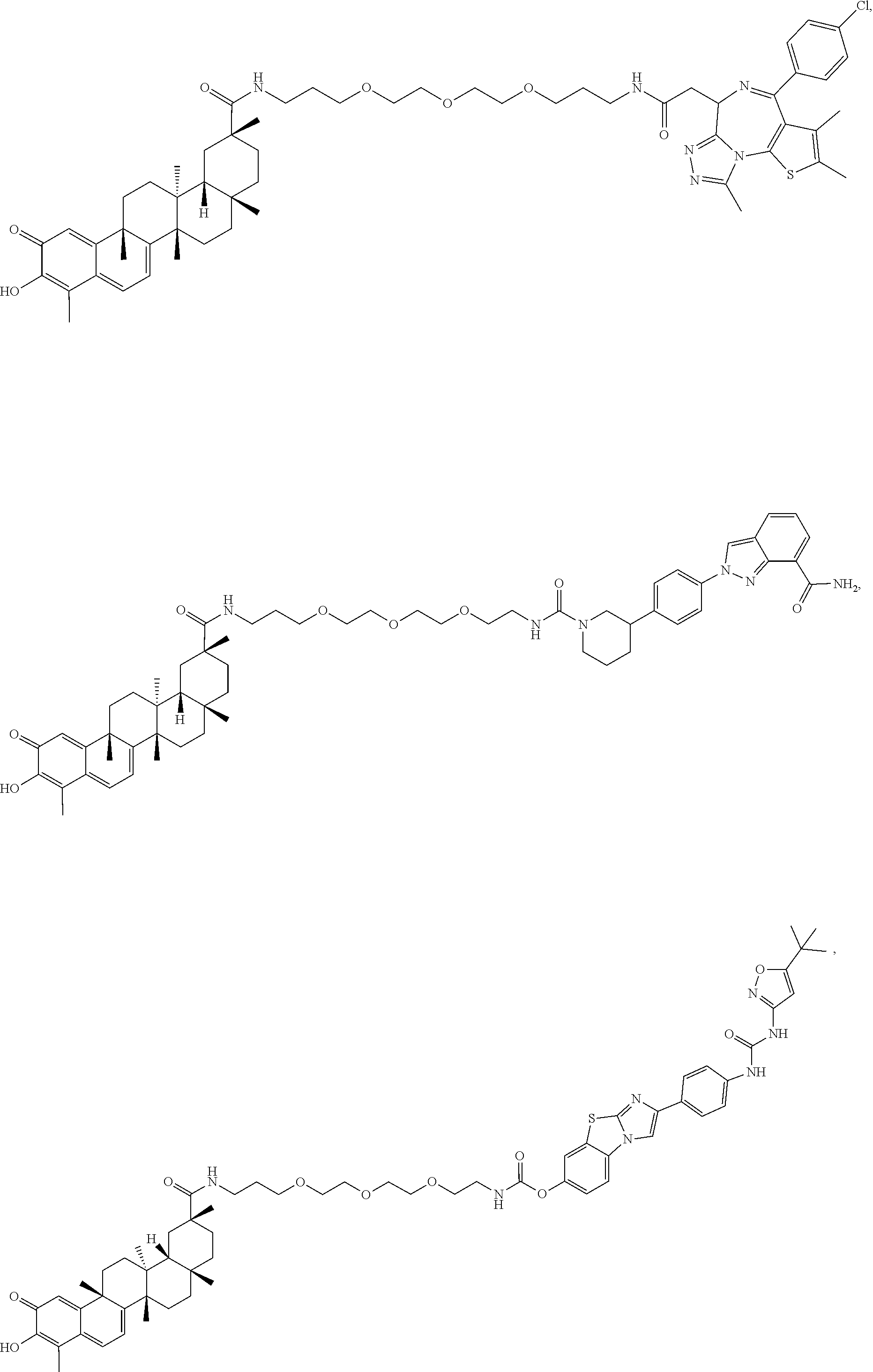 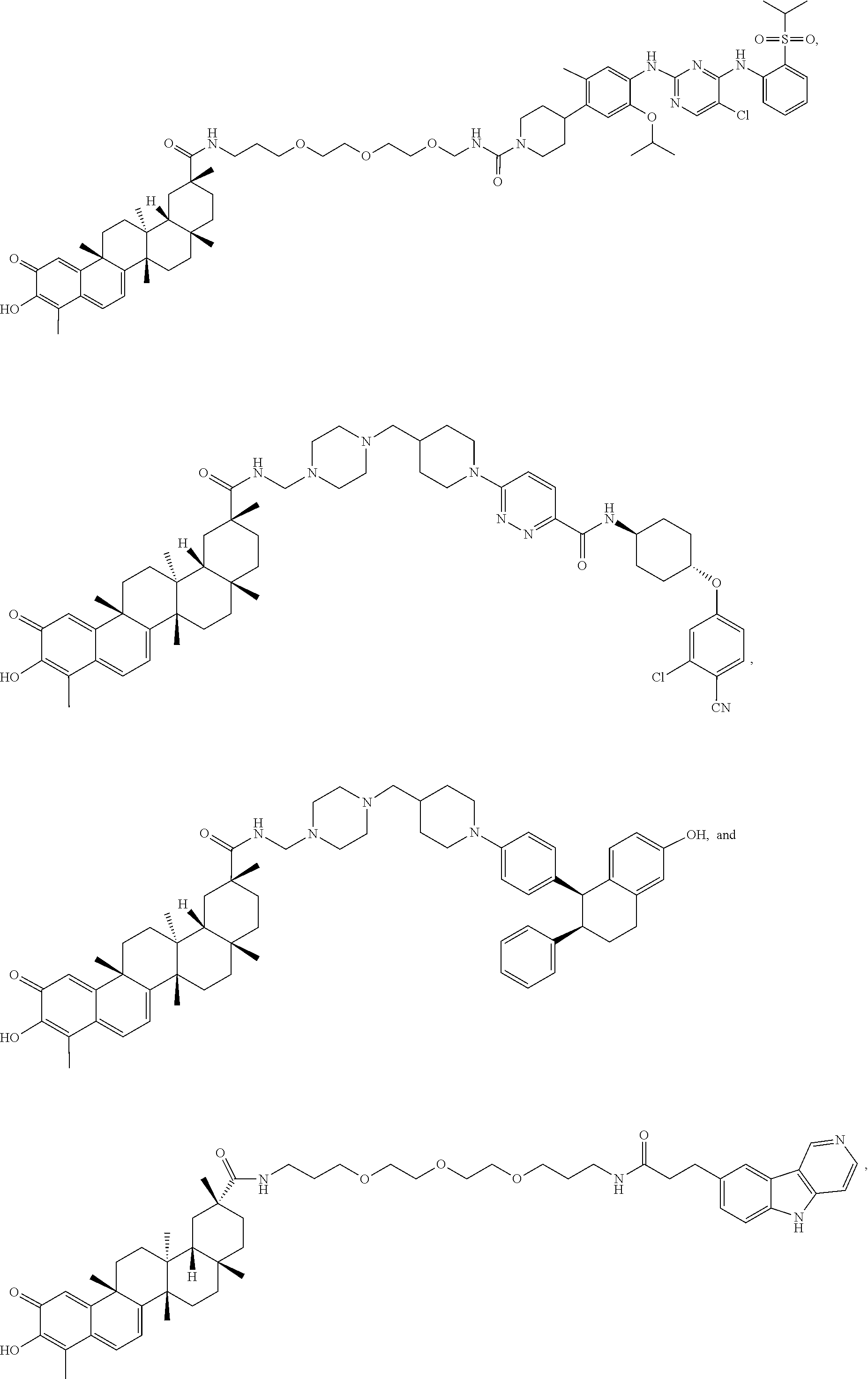
or a pharmaceutically acceptable salt thereof.

20. A pharmaceutical composition comprising a compound of any one of claims 1-19, or a pharmaceutically acceptable salt thereof, and a pharmaceutically acceptable carrier.

21. A method of treating a disease or condition, the method comprising administering to a subject in need thereof a therapeutically effective amount of a compound of any one of claims 1-13, or a pharmaceutically acceptable salt thereof, wherein the protein is implicated in the pathology of the disease or condition.

22. The method of claim 21, wherein the disease or condition is cancer.

23. The method of claim 22, wherein the cancer is selected from bladder cancer, brain cancer, breast cancer, colorectal cancer, cervical cancer, gastrointestinal cancer, genitourinary cancer, head and neck cancer, lung cancer, ovarian cancer, pancreatic cancer, prostate cancer, renal cancer, skin cancer, and testicular cancer. In some embodiments, the cancer is selected from sarcoma, angiosarcoma, fibrosarcoma, rhabdomyosarcoma, liposarcoma, myxoma, rhabdomyoma, fibroma, lipoma, teratoma, lung cancer, bronchogenic carcinoma squamous cell, undifferentiated small cell, undifferentiated large cell, adenocarcinoma, alveolar bronchiolar carcinoma, bronchial adenoma, sarcoma, lymphoma, chondromatous hamartoma, mesothelioma, gastrointestinal cancer, cancer of the esophagus, squamous cell carcinoma, adenocarcinoma, leiomyosarcoma, lymphoma, cancer of the stomach, carcinoma, lymphoma, leiomyosarcoma, cancer of the pancreas, ductal adenocarcinoma, insulinoma, glucagonoma, gastrinoma, carcinoid tumor, vipoma, cancer of the small bowel, adenocarcinoma, lymphoma, carcinoid tumors, Kaposi's sarcoma, leiomyoma, hemangioma, lipoma, neurofibroma, fibroma, cancer of the large bowel or colon, tubular adenoma, villous adenoma, hamartoma, leiomyoma, genitourinary tract cancer, cancer of the kidney adenocarcinoma, Wilm's tumor (nephroblastoma), cancer of the bladder, cancer of the urethra, squamous cell carcinoma, transitional cell carcinoma, cancer of the prostate, cancer of the testis, seminoma, teratoma, embryonal carcinoma, teratocarcinoma, choriocarcinoma, sarcoma, interstitial cell carcinoma, fibroma, fibroadenoma, adenomatoid tumors, lipoma, liver cancer, hepatoma hepatocellular carcinoma, cholangiocarcinoma, hepatoblastoma, angiosarcoma, hepatocellular adenoma, hemangioma, bone cancer, osteogenic sarcoma (osteosarcoma), fibrosarcoma, malignant fibrous histiocytoma, chondrosarcoma, Ewing's sarcoma, malignant lymphoma (reticulum cell sarcoma), malignant giant cell tumor, chordoma, osteochrondroma (osteocartilaginous exostoses), benign chondroma, chondroblastoma, chondromyxofibroma, osteoid osteoma giant cell tumor, nervous system cancer, cancer of the skull, osteoma, hemangioma, granuloma, xanthoma, osteitis deformans, cancer of the meninges meningioma, meningiosarcoma, gliomatosis, cancer of the brain, astrocytoma, medulloblastoma, glioma, ependymoma, germinoma (pinealoma), glioblastoma multiforme, oligodendroglioma, schwannoma, retinoblastoma, congenital tumors, cancer of the spinal cord, neurofibroma, meningioma, glioma, sarcoma, gynecological cancer, cancer of the uterus, endometrial carcinoma, cancer of the cervix, cervical carcinoma, pre tumor cervical dysplasia, cancer of the ovaries, ovarian carcinoma, serous cystadenocarcinoma, mucinous cystadenocarcinoma, unclassified carcinoma, granulosa-theca cell tumor, Sertoli Leydig cell tumor, dysgerminoma, malignant teratoma, cancer of the vulva, squamous cell carcinoma, intraepithelial carcinoma, adenocarcinoma, fibrosarcoma, melanoma, cancer of the vagina, clear cell carcinoma, squamous cell carcinoma, botryoid sarcoma, embryonal rhabdomyosarcoma, cancer of the fallopian tubes, hematologic cancer, cancer of the blood, lymphoma, leukemia, acute myeloid leukemia (AML), chronic myeloid leukemia (CML), acute lymphoblastic leukemia (ALL), chronic lymphoblastic leukemia, chronic lymphocytic leukemia, myeloproliferative diseases, multiple myeloma, myelodysplastic syndrome, Hodgkin's lymphoma, non-Hodgkin's lymphoma (malignant lymphoma), Waldenstrom's macroglobulinemia, skin cancer, malignant melanoma, basal cell carcinoma, squamous cell carcinoma, Kaposi's sarcoma, moles dysplastic nevi, lipoma, angioma, dermatofibroma, keloids, adrenal gland cancer, and neuroblastoma.

24. The method of claim 21, wherein the disease or condition is a neurodegenerative disease or condition.

25. The method of claim 24, wherein the neurodegenerative disease or condition is selected from Huntington's disease (HD), Alzheimer's disease (AD), Parkinson's disease (PD), multiple sclerosis (MS), frontotemporal dementia (FTD), amyotrophic lateral sclerosis (ALS), Lewy body disease, dementia, motor neuron disease (MND), Prion disease, cerebral amyloid angiopathy, vascular cognitive impairment (VCI), hippocampal sclerosis, Binswanger's disease, Creutzfeldt-Jakob disease, cerebral ischemia, cerebrovascular ischemia, brain ischemia, cerebral palsy, chemotherapy-induced brain damage; cisplatin-induced neurotoxicity, diabetic neuropathy; Down's syndrome, epilepsy and post-traumatic epilepsy; Friedreich's ataxia; Hallervorden-Spatz disease; macular degeneration; methanol-induced neurotoxicity; meningitis (aseptic and tuberculous);
Pick's disease; progressive supra-nuclear palsy; radiotherapy-induced brain damage;
senile dementia; schizophrenia; traumatic brain injury (TBI); traumatic spinal injury; viral meningitis; encephalitis, and viral encephalitis.

26. A method of treating cancer, the method comprising administering a subject in need thereof:
(i) a therapeutically effective amount of celastrol having formula: 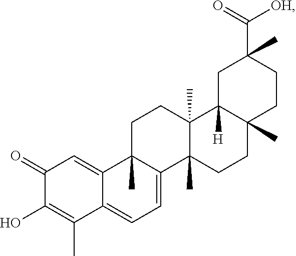
or a pharmaceutically acceptable salt thereof, and
(ii) a therapeutically effective amount of a bromodomain inhibitor, or a pharmaceutically acceptable salt thereof.

27. The method of claim 26, wherein the cancer is selected from breast cancer, glioma, and pediatric glioma.

28. The method of claim 26, wherein the bromodomain inhibitor is selected from BRD2 inhibitor, BRD3 inhibitor, BRD4 inhibitor, and BRDT inhibitor.

29. The method of claim 26, wherein the bromodomain inhibitor is selected from I-BET 151, JQ1, I-BET 762, OTX-015, TEN-010, CPI-203, CPI-0610, olinone, RVX-208, ABBV-744, LY294002, AZD5153, MT-1, and MS645, or a pharmaceutically acceptable salt thereof.

30. The method of claim 26, wherein the bromodomain inhibitor is JQ-1 having formula: 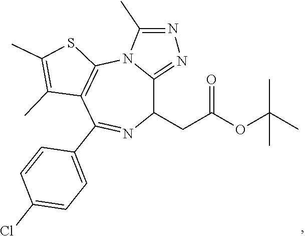 [SMILES: Cc1sc2c(c1C)C(c1ccc(Cl)cc1)=NC(CC(=O)OC(C)(C)C)c1nnc(C)n1-2]
or a pharmaceutically acceptable salt thereof.
